(19) United States
(12) Patent Application Publication
(10) Pub. No.: US 2010/0168075 A1
(43) Pub. Date: Jul. 1, 2010

(54) Novel 2-Azetidinone Derivatives As Cholesterol Absorption Inhibitors For The Treatment Of Hyperlipidaemic Conditions

(75) Inventors: Mikael Dahlstrom, Molndal (SE); Fana Hunegnaw, Molndal (SE); Malin Lemurell, Molndal (SE); Peter Nordberg, Molndal (SE); Tore Skjaret, Molndal (SE); Ingemar Starke, Molndal (SE)

Correspondence Address: Pepper Hamilton LLP, 400 Berwyn Park, 899 Cassatt Road, Berwyn, PA, 19312-1183, US

(73) Assignee: ASTRAZENECA AB, Sodertalje (SE)

(21) Appl. No.: 11/993,470

(22) PCT Filed: Jun. 21, 2006
(86) PCT No.: PCT/SE06/00764

§ 371 Date: Dec. 21, 2007

(30) Foreign Application Priority Data
Jun. 22, 2005 (SE) 0501466-7

(51) Int. Cl. A61K 31/397; C07D 205/09; A61P 3/06; A61P 9/10; A61P 25/28

(52) U.S. Cl. 514/210.02; 540/360

(57) ABSTRACT

The application relates to novel 2-azetidinone derivatives of formula (I) and pharmaceutically acceptable salts, solvates and prodrugs thereof. The compounds are cholesterol absorption inhibitors and are useful in the treatment of hyperlipidaemic conditions, including atherosclerosis, Alzheimers' disease and cholesterol associated tumours. The application also relates to pharmaceutical formulations comprising such compounds and to processes for their preparation.

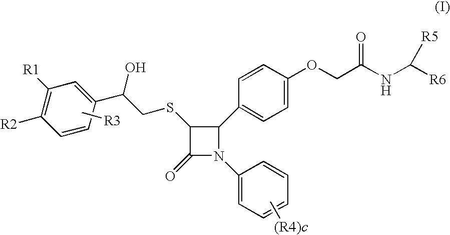

This invention relates to 2-azetidinone derivatives, or pharmaceutically acceptable salts, solvates, solvates of such salts and prodrugs thereof. These 2-azetidinones possess cholesterol absorption inhibitory activity and are accordingly of value in the treatment of disease states associated with hyperlipidaemic conditions. They are therefore useful in methods of treatment of a warm-blooded animal, such as man. The invention also relates to processes for the manufacture of said 2-azetidinone derivatives, to pharmaceutical compositions containing them and to their use in the manufacture of medicaments to inhibit cholesterol absorption in a warm-blooded animal, such as man. A further aspect of this invention relates to the use of the compounds of the invention in the treatment of dyslipidemic conditions.

Atherosclerotic coronary artery disease is a major cause of death and morbidity in the western world as well as a significant drain on healthcare resources. It is well-known that hyperlipidaemic conditions associated with elevated concentrations of total cholesterol and low density lipoprotein (LDL) cholesterol are major risk factors for cardiovascular atherosclerotic disease (for instance “Coronary Heart Disease: Reducing the Risk; a Worldwide View” Assman G., Carmena R. Cullen P. et al; Circulation 1999, 100, 1930-1938 and “Diabetes and Cardiovascular Disease: A Statement for Healthcare Professionals from the American Heart Association” Grundy S, Benjamin I., Burke G., et al; Circulation, 1999, 100, 1134-46).

The concentration of plasma cholesterol depends on the integrated balance of endogenous and exogenous pathways of cholesterol metabolism. In the endogenous pathway, cholesterol is synthesized by the liver and extra hepatic tissues and enters the circulation as lipoproteins or is secreted into bile. In the exogenous pathway cholesterol from dietary and biliary sources is absorbed in the intestine and enters the circulation as component of chylomicrons. Alteration of either pathway will affect the plasma concentration of cholesterol.

The precise mechanism by which cholesterol is absorbed from the intestine is however not clear. The original hypothesis has been that cholesterol is crossing the intestine by unspecific diffusion. But more recent studies are suggesting that there are specific transporters involved in the intestinal cholesterol absorption. (See for instance New molecular targets for cholesterol-lowering therapy Izzat, N,N., Deshazer, M. E. and Loose-Mitchell D. S. JPET 293:315-320, 2000.)

A clear association between reduction of total cholesterol and (LDL) cholesterol and decreased instance of coronary artery disease has been established, and several classes of pharmaceutical agents are used to control serum cholesterol. There major options to regulate plasma cholesterol include (i) blocking the synthesis of cholesterol by agents such as HMG-CoA reductase inhibitors, for example statins such as simvastatin and fluvastatin, which also by up-regulation of LDL-receptors will promote the cholesterol removal from the plasma; (ii) blocking the bile acid reabsorption by specific agents resulting in increased bile acid excretion and synthesis of bile acids from cholesterol with agents such as bile acid binders, such as resins e.g. cholestyramine and cholestipol; and (iii) by blocking the intestinal uptake of cholesterol by selective cholesterol absorption inhibitors. High density lipoprotein (HDL) elevating agents such as fibrates and nicotinic acid analogues have also been employed.

Even with the current diverse range of therapeutic agents, a significant proportion of the hypercholesterolaemic population is unable to reach target cholesterol levels, or drug interactions or drug safety preclude the long term use needed to reach the target levels. Therefore there is still a need to develop additional agents that are more efficacious and are better tolerated.

Compounds possessing such cholesterol absorption inhibitory activity have been described, see for instance the compounds described in WO 93/02048, WO 94/17038, WO 95/08532, WO 95/26334, WO 95/35277, WO 96/16037, WO 96/19450, WO 97/16455, WO 02/50027, WO 02/50060, WO 02/50068, WO 02/50090, WO 02/66464, WO 04/000803, WO 04/000804, WO04/000805, WO04/01993, WO04/010948, WO04/043456 WO 04/043457, WO 04/081002, WO05/000353, WO05/021495, WO05/021497, WO05/033100.

The present invention is based on the discovery that certain 2-azetidinone derivatives surprisingly inhibit cholesterol absorption. Such properties are expected to be of value in the treatment of disease states associated with hyperlipidaemic conditions. The compounds of the present invention are not disclosed in any of the above applications and we have surprisingly found that the compounds of the present invention possess beneficial efficacious, metabolic and toxicological profiles that make them particularly suitable for in vivo administration to a warm blooded animal, such as man. In particular certain compounds of the present invention have a low degree of absorption compared to compounds of the prior art whilst retaining their ability to inhibit cholesterol absorption.

Accordingly there is provided a compound of formula (I):

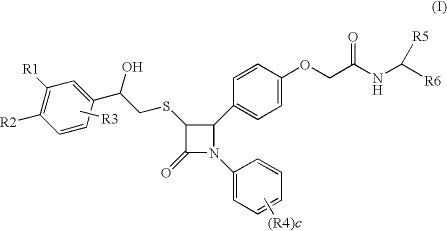

wherein:
R¹ and R² are hydrogen, C₁₋₆alkyl, C₃₋₆cycloalkyl or aryl; wherein said C₁₋₆alkyl may be optionally substituted by one or more hydroxy, amino, guanidino, carbamoyl, carboxy, C₁₋₆alkoxy, N-(C₁₋₆alkyl)amino, N,N-(C₁₋₆alkyl)₂amino, C₁-C₆alkylcarbonylamino C₁₋₆alkylS(O)ₐ wherein a is 0-2, C₃₋₆cycloalkyl or aryl; and wherein any aryl group may be optionally substituted by one or two substituents selected from halo, hydroxy, C₁₋₆alkyl or C₁₋₆alkoxy; or R¹ and R² form a 5, 6 or 7 membered carbocyclic or heterocyclic ring fused with the phenyl wherein the heterocyclic ring contains 1-2 heteroatoms selected from oxygen nitrogen or sulphur.
R³ is hydrogen, halo, hydroxy, C₁₋₆alkyl, C₁₋₆alkoxy or C₁₋₆alkylS(O)ₐ wherein a is 0 to 2; wherein R³ is independently optionally substituted on carbon by one or more halo, C₁₋₆alkoxy and hydroxy;
R⁴ is halo, nitro, cyano, hydroxy, amino, carboxy, formyl, carbamoyl, carbamoyloxy, mercapto, sulphamoyl, C₁₋₆alkyl, C₂₋₆alkenyl, C₂₋₆alkenyloxy, C₂₋₆alkynyl, C₁₋₆alkoxy, C₁₋₆alkanoyl, C₁₋₆alkanoyloxy, N-(C₁₋₄alkyl)amino, N,N-(C₁₋₆alkyl)₂-amino, C₁₋₆alkanoylamino, C₁₋₆alkanoyl-N-(C₁₋₆alkyl)amino, C₁₋₆alkylsulphonylamino, C₁₋₆alkylsulphonyl-N-(C₁₋₆alkyl)amino, N-(C₁₋₆alkyl)carbamoyl, N,N-(C₁₋₆alkyl)₂carbamoyl, N-(C₁₋₆alkyl)carbamoyloxy, N,N-(C₁₋₆alkyl)₂carbamoyloxy, C₁₋₆alkylS(O) wherein a is 0 to 2, C₁₋₆alkoxycarbonyl, C₁₋₆alkoxycarbonylamino, C₁₋₆alkoxycarbonyl-N-(C₁₋₆alkyl)amino, C₁₋₆alkoxycarbonyloxy, C₁₋₆alkoxycarbonylamino, ureido, N′-(C₁₋₆alkyl)ureido, N-(C₁₋₆alkyl)ureido, N′,N′-(C₁₋₆alkyl)₂ureido, N′-(C₁₋₆alkyl)-N-(C₁₋₆alkyl)ureido, N′,N′-(C₁₋₆alkyl)₂-N-(C₁₋₆alkyl)ureido, N-(C₁₋₆alkyl)sulphamoyl, N,N-(C₁₋₆alkyl)₂sulphamoyl or phenyl; wherein R⁴ is independently optionally substituted on carbon by one or more halo, C₁₋₆alkoxy, hydroxy, amino, carboxy, C₁₋₆alkoxycarbonyl, carbamoyl, N-(C₁₋₆alkyl)carbamoyl, N,N-(C₁₋₆alkyl)₂-carbamoyl, C₁₋₆alkanoylamino, C₁₋₆alkanoyl-N-(C₁₋₆alkyl)amino, phenyl, phenoxy, benzoyl, phenylC₁₋₆alkyl and phenylC₁₋₆alkoxy;

R⁵ is hydrogen, halo, nitro, cyano, hydroxy, amino, mercapto, sulphamoyl, hydroxyaminocarbonyl, C₂₋₁₀alkenyl, C₂₋₁₀-alkynyl, C₁₋₁₀alkoxy, C₁₋₁₀alkoxycarbonyl, C₁₋₁₀alkanoyl, C₁₋₁₀alkanoyloxy, N-(C₁₋₁₀alkyl)amino, N,N-(C₁₋₁₀alkyl)₂amino, N,N,N-(C₁₋₁₀alkyl)₃ammonio, C₁₋₁₀alkanoylamino, C₁₋₁₀alkylS(O)ₐ wherein a is 0 to 2, N-(C₁₋₁₀alkyl)sulphamoyl, N,N-(C₁₋₁₀alkyl)₂sulphamoyl, N-(C₁₋₁₀alkyl)sulphamoylamino, N,N-(C₁₋₁₀alkyl)₂sulphamoylamino, C₁₋₁₀alkoxycarbonylamino, carbocyclyl, carbocyclylC₁₋₁₀alkyl, heterocyclyl, heterocyclylC₁₋₁₀alkyl, carbocyclyl-(C₁₋₁₀alkylene)ₑ-R²⁹-(C₁₋₁₀alkylene)_{f}-, heterocyclyl-(C₁₋₁₀alkylene)_{g}-R³⁰-(C₁₋₁₀alkylene)ₕ-, carboxy, sulpho, sulphino, phosphono, —P(O)(OR³¹)(OR³²), —P(O)(OH)(OR³¹), —P(O)(OH)(R³¹) or —P(O)(OR³¹)(R³²) wherein R³¹ and R³² are independently selected from C₁₋₆alkyl; wherein R⁵ may be optionally substituted on carbon by one or more substituents selected from R³³; and wherein if said heterocyclyl contains an —NH— group, that nitrogen may be optionally substituted by a group selected from R³⁴; or R⁵ is a group of formula (IA):

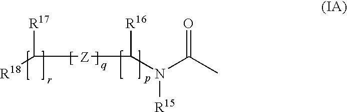

wherein:

Z is —N(R³⁵)—, —N(R³⁵)C(O)—, —O—, and —S(O)ₐ—; wherein a is 0-2 and R³⁵ is hydrogen or C₁₋₄alkyl;

R¹⁵ is hydrogen or C₁₋₄alkyl;

R¹⁶ and R¹⁷ are independently selected from hydrogen, halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, N,N,N-(C₁₋₁₀alkyl)₃ammonio C₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, N-(C₁₋₆alkyl)sulphamoyl, N,N-(C₁₋₆alkyl)₂sulphamoyl, carbocyclyl, heterocyclyl, sulpho, sulphino, amidino, phosphono, —P(O)(OR³⁶)(OR³⁷), —P(O)(OH)(OR³⁶), —P(O)(OH)(R³⁶) or —P(O)(OR³⁶)(R³⁷), wherein R³⁶ and R³⁷ are independently selected from C₁₋₆alkyl; wherein R¹⁶ and R¹⁷ may be independently optionally substituted on carbon by one or more substituents selected from R³⁸; and wherein if said heterocyclyl contains an —NH— group, that nitrogen may be optionally substituted by a group selected from R³⁹;

R¹⁸ is selected from hydrogen, halo, nitro, cyano, hydroxy, amino, carbamoyl, mercapto, sulphamoyl, hydroxyaminocarbonyl, C₁₋₁₀alkyl, C₂₋₁₀alkenyl, C₂₋₁₀alkynyl, C₁₋₁₀alkoxy, C₁₋₁₀alkanoyl, C₁₋₁₀alkanoyloxy, N-(C₁₋₁₀alkyl)amino, N,N-(C₁₋₁₀alkyl)₂amino, N,N,N-(C₁₋₁₀alkyl)₃ammonio C₁₋₁₀alkanoylamino, N-(C₁₋₁₀alkyl)carbamoyl, C₁₋₁₀alkoxycarbonyl, N,N-(C₁₋₁₀alkyl)₂carbamoyl, C₁₋₁₀alkylS(O)ₐ wherein a is 0 to 2, N-(C₁₋₁₀alkyl)sulphamoyl, N,N-(C₁₋₁₀alkyl)₂sulphamoyl, N-(C₁₋₁₀alkyl)sulphamoylamino, N,N-(C₁₋₁₀alkyl)₂sulphamoylamino, carbocyclyl, carbocyclylC₁₋₁₀alkyl, heterocyclyl, heterocyclylC₁₋₁₀alkyl, carbocyclyl-(C₁₋₁₀alkylene)ₐ-R⁴⁰-(C₁₋₁₀alkylene)_{f}- or heterocyclyl-(C₁₋₁₀alkylene)_{g}-R⁴¹-(C₁₋₁₀alkylene)ₕ-, carboxy, sulpho, sulphino, phosphono, —P(O)(OR⁴²)(OR⁴³), —P(O)(OH)(OR⁴²), —P(O)(OH)(R⁴²) or —P(O)(OR⁴²)(R⁴³) wherein R⁴² and R⁴³ are independently selected from C₁₋₆alkyl; wherein R¹⁸ may be optionally substituted on carbon by one or more substituents selected from R⁴⁴; and wherein if said heterocyclyl contains an —NH— group, that nitrogen may be optionally substituted by a group selected from R⁴⁵; R¹⁸ and R¹⁵ may be linked to form a 4-7 membered ring; or R¹⁸ is a group of formula

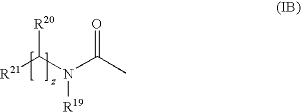

wherein:

R¹⁹ is selected from hydrogen or C₁₋₄alkyl;

R²⁰ is selected from hydrogen, halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, N,N,N-(C₁₋₁₀alkyl)₃ammonio, C₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, N-(C₁₋₆alkyl)sulphamoyl, N,N-(C₁₋₆alkyl)₂sulphamoyl, carbocyclyl, heterocyclyl, sulpho, sulphino, amidino, phosphono, —P(O)(OR⁴⁶)(OR⁴⁷), —P(O)(OH)(OR⁴⁶), —P(O)(OH)(R⁴⁶) or —P(O)(OR⁴⁶)(R⁴⁷), wherein R⁴⁶ and R⁴⁷ are independently selected from C₁₋₆alkyl; where R²⁰ may be independently optionally substituted on carbon by one or more substituents selected from R⁴⁸; and wherein if said heterocyclyl contains an —NH— group, that nitrogen may be optionally, substituted by a group selected from R⁴⁹;

R²¹ is selected from halo, nitro, cyano, hydroxy, amino, carbamoyl, mercapto, sulphamoyl, hydroxyaminocarbonyl, C₁₋₁₀alkenyl, C₂₋₁₀alkynyl, C₁₋₁₀alkoxy, C₁₋₁₀alkoxycarbonyl, C₁₋₁₀alkanoyl, C₁₋₁₀alkanoyloxy, N-(C₁₋₁₀alkyl)amino, N,N-(C₁₋₁₀alkyl)₂amino, N,N,N-(C₁₋₄₀alkyl)₃ammonio, C₁₋₁₀alkanoylamino, N-(C₁₋₁₀alkyl)carbamoyl, N,N-(C₁₋₁₀alkyl)₂carbamoyl, C₁₋₁₀alkylS(O)ₐ wherein a is 0 to 2, N-(C₁₋₁₀alkyl)sulphamoyl, N,N-(C₁₋₁₀alkyl)₂sulphamoyl, N-(C₁₋₁₀alkyl)sulphamoylamino, N,N-(C₁₋₁₀alkyl)₂sulphamoylamino, C₁₋₁₀alkoxycarbonylamino, carbocyclyl, carbocyclylC₁₋₁₀alkyl, heterocyclyl, heterocyclylC₁₋₁₀alkyl, carbocyclyl-(C₂₋₁₀alkylene)ₑ-R⁵⁰-(C₁₋₁₀alkylene)_{f}-, heterocyclyl-(C₁₋₁₀alkylene)_{g}-R⁵¹-(C₁₋₁₀alkylene)ₕ-, carboxy, sulpho, sulphino, phosphono, —P(O)(OR⁵²)(OR⁵³), —P(O)(OH)(OR⁵²), —P(O)(OH)(R⁵²) or —P(O)(OR⁵³)(R⁵³) wherein R⁵² and R⁵³ are independently selected from C₁₋₆alkyl; wherein R²¹ may be independently optionally substituted on carbon by one or more R⁵⁴; and wherein if said heterocyclyl contains an —NH— group, that nitrogen may be optionally substituted by a group selected from R⁵⁵;

p is 1-3; wherein the values of R¹⁶ may be the same or different;

q is 0-1;

r is 0-3; wherein the values of R¹⁷ may be the same or different;

m is 0-2; wherein the values of R¹³ may be the same or different;

n is 1-2; wherein the values of R⁹ may be the same or different;

z is 0-3; wherein the values of R²⁰ may be the same or different;

R²⁵, R²⁷, R³³, R³⁸, R⁴⁴, R⁴⁸ and R⁵⁴ are independently selected from halo, nitro, cyano, hydroxy, amino, carbamoyl, mercapto, sulphamoyl, hydroxyaminocarbonyl, C₁₋₁₀alkenyl, C₂₋₁₀alkynyl, C₁₋₁₀alkoxy, C₁₋₁₀alkanoyl, C₁₋₁₀alkanoyloxy, C₁₋₁₀alkoxycarbonyl, N-(C₁₋₁₀alkyl)amino, N,N-(C₁₋₁₀alkyl)₂amino, N,N,N-(C₁₋₁₀alkyl)₃ammonio, C₁₋₁₀alkanoylamino, N-(C₁₋₁₀alkyl)carbamoyl, N,N-(C₁₋₁₀alkyl)₂carbamoyl, C₁₋₁₀alkylS(O)ₐ wherein a is 0 to 2, N-(C₁₋₁₀alkyl)sulphamoyl, N,N-(C₁₋₁₀alkyl)₂sulphamoyl, N-(C₁₋₁₀alkyl)sulphamoylamino, N,N-(C₁₋₁₀alkyl)₂sulphamoylamino, C₁₋₁₀alkoxycarbonylamino, carbocyclyl, carbocyclylC₁₋₁₀alkyl, heterocyclyl, heterocyclylC₁₋₁₀alkyl, carbocyclyl-(C₁₋₁₀alkylene)ₑ-R⁵⁶-(C₁₋₁₀alkylene)_{f}-, heterocyclyl-(C₂₋₁₀alkylene)_{g}-R⁵⁷-(C₁₋₁₀alkylene)ₕ-, carboxy, sulpho, sulphino, amidino, phosphono, —P(O)(OR⁵⁸)(OR⁵⁹), —P(O)(OH)(OR⁵⁸), —P(O)(OH)(R⁵⁸) or —P(O)(OR⁵⁹)(R⁵⁹), wherein R⁵⁸ and R⁵⁹ are independently selected from C₁₋₆alkyl; wherein R²³, R²⁵, R²⁷, R³³, R³⁸, R⁴⁴, R⁴⁸ and R⁵⁴ may be independently optionally substituted on carbon by one or more R⁶⁰; and wherein if said heterocyclyl contains an —NH— group, that nitrogen may be optionally substituted by a group selected from R⁶¹;

R³⁴, R³⁹, R⁴⁵, R⁴⁹, R⁵⁵ and R⁶¹ are independently selected from C₁₋₆alkyl, C₁₋₆alkanoyl, C₁₋₆alkylsulphonyl, sulphamoyl, N-(C₁₋₆alkyl)sulphamoyl, N,N-(C₁₋₆alkyl)₂sulphamoyl, C₁₋₆alkoxycarbonyl, carbamoyl, N-(C₁₋₆alkyl)carbamoyl, N,N-(C₁₋₆alkyl)₂carbamoyl, benzyl, phenethyl, benzoyl, phenylsulphonyl and phenyl;

R²⁹, R³⁰, R⁴⁰, R⁴¹, R⁵⁰, R⁵¹, R⁵⁶ and R⁵⁷ are independently selected from —O—, —NR⁶²—, —S(O)ₓ—, —NR⁶²C(O)NR⁶³—, —NR⁶²C(S)NR⁶³—, —OC(O)N═C—, —NR⁶²C(O)— or —C(O)NR⁶²—; wherein R⁶² and R⁶³ are independently selected from hydrogen or C₁₋₆alkyl, and x is 0-2;

R⁶⁰ is selected from halo, hydroxy, cyano, carbamoyl, ureido, amino, nitro, carboxy, carbamoyl, mercapto, sulphamoyl, trifluoromethyl, trifluoromethoxy, methyl, ethyl, methoxy, ethoxy, vinyl, allyl, ethynyl, methoxycarbonyl, formyl, acetyl, formamido, acetylamino, acetoxy, methylamino, dimethylamino, N-methylcarbamoyl, N,N-dimethylcarbamoyl, methylthio, methylsulphinyl, mesyl, N-methylsulphamoyl and N,N-dimethylsulphamoyl; and

e, f, g and h are independently selected from 0-2;

R⁶ is hydrogen, alkyl, c-alkyl or aryl;
c=1, 2, 3, 4 or 5;
or a pharmaceutically acceptable salt, solvate, solvate of such a salt or a prodrug thereof;
provided that compounds of formula (I) are not compounds of formula A

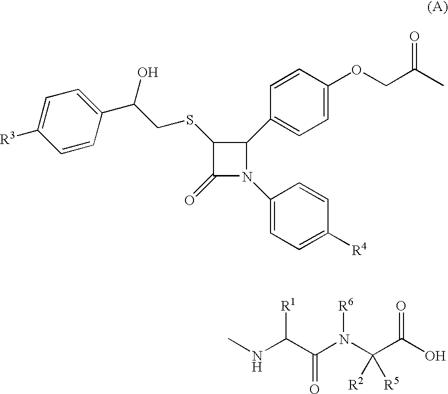

wherein:

R¹ is hydrogen, C₁₋₆alkyl, C₃₋₆cycloalkyl or aryl; wherein said C₁₋₆alkyl may be optionally substituted by one or more hydroxy, amino, guanidino, carbamoyl, carboxy, C₁₋₆alkoxy, N-(C₁₋₆alkyl)amino, N,N-(C₁₋₆alkyl)₂amino, C₁-C₆alkylcarbonylamino C₁₋₆alkylS(O)ₐ wherein a is 0-2, C₃₋₆cycloalkyl or aryl; and wherein any aryl group may be optionally substituted by one or two substituents selected from halo, hydroxy, C₁₋₆alkyl or C₁₋₆alkoxy;

R² and R⁵ are independently hydrogen, a branched or unbranched C₁₋₆alkyl, C₃₋₆cycloalkyl or aryl; wherein said C₁₋₄alkyl may be optionally substituted by one or more hydroxy, amino, guanidino, cyano, carbamoyl, carboxy, C₁₋₆alkoxy, aryl C₁₋₆alkoxy,(C1-C4 alkyl)₃Si, N-(C₁₋₆alkyl)amino, N,N-(C₁₋₆alkyl)₂-amino, C₁₋₆alkylS(O)ₐ, C₁₋₆alkanoylamino, C₃₋₆cycloalkyl, aryl or aryl C₁₋₆alkylS(O)ₐ, wherein a is 0-2; and wherein any aryl group may be optionally substituted by one or two substituents selected from halo, hydroxy, C₁₋₆alkyl or C₁₋₆alkoxy;
R³ is hydrogen, alkyl, halo, C₁₋₆alkoxy or C₁₋₆alkylS—;
R⁴ is hydrogen, C₁₋₆alkyl, halo or C₁₋₆alkoxy;
R⁶ is hydrogen, C₁₋₆alkyl, or arylC₁₋₆alkyl;
wherein R⁵ and R² may form a ring with 2-7 carbon atoms and wherein R⁶ and R² may form a ring with 3-6 carbon atoms;
or N-{[4-((2R,3R)-3-{[2-(4-fluorophenyl)-2-hydroxyethyl]thio}-4-oxo-1-phenylazetidin-2-yl)phenoxy]acetyl}glycine.

In one aspect of the invention it is provided for a compound of formula I2:

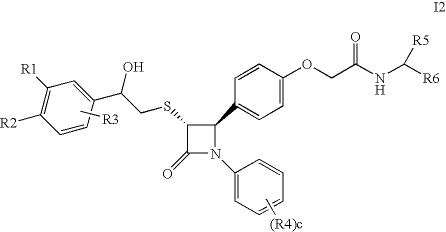

wherein variable groups are defined above as for formula (I). What is said further for formula (I) will, apart from the process schemes below, apply also to formula (I2).

In one aspect of the invention R¹ is hydrogen. According to another aspect of the invention,

R² is hydrogen, halo, C₁₋₄alkoxy, C₁₋₄ alkyl, C₁₋₄ thioalkyl, or R¹ and R² form a 5, 6 or 7 membered carbocyclic or heterocyclic ring fused with the phenyl wherein the heterocyclic ring contains 1-2 heteroatoms selected from oxygen nitrogen or sulphur. According to a further aspect of the invention, R³ is hydrogen, halo, C₁₋₄ alkyl or C₁₋₄ alkoxy. According to yet another aspect of the invention R⁴ is hydrogen, halo, C₁₋₄ alkyl or C₁₋₄ alkoxy. According to a further aspect of the invention R⁵ is C₁₋₁₀alkyl optionally substituted by one or more substituents selected from R³³, carboxy, sulpho, sulphino, phosphono, —P(O)(OR³¹)(OR³²), —P(O)(OH)(OR³¹), —P(O)(OH)(R³¹) or —P(O)(OR³¹)(R³²) wherein R³¹ and R³² are independently selected from C₁₋₆alkyl or R⁵ is a group of formula (IA):

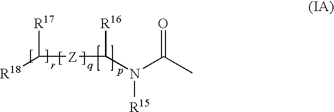

According to another aspect of the invention R⁶ is hydrogen, C₁₋₄ alkyl, C₃₋₆ cycloalkyl or aryl.

According to an aspect of the invention R¹ and R² forms a five-membered ring containing one oxygen or a six-membered ring containing two oxygens.

The invention further provides for one or more compounds chosen from:
(3R)-3-[(N-{[4-((2R,3R)-1-(4-fluorophenyl)-3-{[2-(4-fluorophenyl)-2-hydroxyethyl]thio}-4-oxoazetidin-2-yl)phenoxy]acetyl}glycyl)amino]-4-phenylbutanoic acid;
3-cyclohexyl-3-[(N-{[4-((2R,3R)-1-(4-fluorophenyl)-3-{[2-hydroxy-2-(4-methoxyphenyl)ethyl]thio}-4-oxoazetidin-2-yl)phenoxy]acetyl}glycyl)amino]propanoic acid;
2-[(N-{[4-((2R,3R)-1-(4-fluorophenyl)-3-{[2-(4-fluorophenyl)-2-hydroxyethyl]thio}-4-oxoazetidin-2-yl)phenoxy]acetyl}glycyl)amino]ethanesulfonic acid;
N⁶-(N-{[4-((2R,3R)-1-(4-fluorophenyl)-3-{[2-(4-fluorophenyl)-2-hydroxyethyl]thio}-4-oxoazetidin-2-yl)phenoxy]acetyl}glycyl)-D-lysine;
N-{2-[(N-{[4-((2R,3R)-1-(4-fluorophenyl)-3-{[2-(4-fluorophenyl)-2-hydroxyethyl]thio}-4-oxoazetidin-2-yl)phenoxy]acetyl}glycyl)amino]ethyl}-D-valine;
3-[(N-{[4-((2R,3R)-1-(4-fluorophenyl)-3-{[2-(4-fluorophenyl)-2-hydroxyethyl]thio}-4-oxoazetidin-2-yl)phenoxy]acetyl}glycyl)amino]-4,4-dimethylpentanoic acid;
N-[2-({[4-((2R,3R)-1-(4-fluorophenyl)-3-{[2-(4-fluorophenyl)-2-hydroxyethyl]thio}-4-oxoazetidin-2-yl)phenoxy]acetyl}amino)ethyl]-D-valine; and
N-{[4-((2R,3R)-1-(4-fluorophenyl)-3-{[(2R or S)-2-(4-fluorophenyl)-2-hydroxyethyl]thio}-4-oxoazetidin-2-yl)phenoxy]acetyl}glycyl-3-cyclohexyl-N-(2-sulfoethyl)-D-alaninamide.

In this specification the term “alkyl” includes both straight and branched chain alkyl groups but references to individual alkyl groups such as “propyl” are specific for the straight chain version only. For example, “C₁₋₄alkyl” and “C₁₋₄alkyl” include propyl, isopropyl and t-butyl. However, references to individual alkyl groups such as ‘propyl’ are specific for the straight chained version only and references to individual branched chain alkyl groups such as ‘isopropyl’ are specific for the branched chain version only. A similar convention applies to other radicals, for example “phenylC₁₋₆alkyl” would include benzyl, 1-phenylethyl and 2-phenylethyl. The term “halo” refers to fluoro, chloro, bromo and iodo.

Where optional substituents are chosen from “one or more” groups it is to be understood that this definition includes all substituents being chosen from one of the specified groups or the substituents being chosen from two or more of the specified groups.

The term “aryl” refers to a 4-10 membered aromatic mono or bicyclic ring containing 0 to 5 heteroatoms independently selected from nitrogen, oxygen or sulphur. Examples of aryls include phenyl, pyrrolyl, furanyl, imidazolyl, triazolyl, tetrazolyl, pyrazinyl, pyrimidinyl, pyridazinyl, pyridyl, isoxazolyl, oxazolyl, 1,2,4 oxadiazolyl, isothiazolyl, thiazolyl, 1,2,4-triazolyl, thienyl, naphthyl, benzofuranyl, benzimidazolyl, benzthienyl, benzthiazolyl, benzisothiazolyl, benzoxazolyl, benzisoxazolyl, 1,3-benzodioxolyl, indolyl, pyridoimidazolyl, pyrimidoimidazolyl, quinolyl, isoquinolyl, quinoxalinyl, quinazolinyl, phthalazinyl, cinnolinyl and naphthyridinyl. Particularly “aryl” refers to phenyl, thienyl, pyridyl, imidazolyl or indolyl. The term “aryl” includes both unsubstituted and substituted aromatic rings.

Examples of “C₁₋₆alkoxy” include methoxy, ethoxy and propoxy. Examples of “C₁₋₆alkylS(O)ₐ wherein a is 0 to 2” include methylthio, ethylthio, methylsulphinyl, ethylsulphinyl, mesyl and ethylsulphonyl. Examples of “N-(C₁₋₆alkyl)amino” include methylamino and ethylamino. Examples of “N,N-(C₁₋₆alkyl)₂amino” include di-N-methylamino, di-(N-ethyl)amino and N-ethyl-N-methylamino. “C₃₋₆cycloalkyl” refers to cyclopropyl, cyclobutyl, cyclopentyl and cyclohexyl.

A suitable pharmaceutically acceptable salt of a compound of the invention, or other compounds disclosed herein, is, for example, an acid-addition salt of a compound of the invention which is sufficiently basic, for example, an acid-addition salt with, for example, an inorganic or organic acid, for example hydrochloric, hydrobromic, sulphuric, phosphoric, trifluoroacetic, citric, acetate or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

The compounds of the formula (I), or other compounds disclosed herein, may be administered in the form of a pro-drug which is broken down in the human or animal body to give a compound of the formula (I). Examples of pro-drugs include in vivo hydrolysable esters and in vivo hydrolysable amides of a compound of the formula (I). An in vivo hydrolysable ester of a compound of the formula (I), or other compounds disclosed herein, containing carboxy or hydroxy group is, for example, a pharmaceutically acceptable ester which is hydrolysed in the human or animal body to produce the parent acid or alcohol. Suitable pharmaceutically acceptable esters for carboxy include C₁₋₆alkoxymethyl esters for example methoxymethyl, C₁₋₆alkanoyloxymethyl esters for example pivaloyloxymethyl, phthalidyl esters, C₃₋₈cycloalkoxycarbonyloxyC₁₋₆alkyl esters for example 1-cyclohexylcarbonyloxyethyl; 1,3-dioxolen-2-onylmethyl esters for example 5-methyl-1,3-dioxolen-2-onylmethyl; and C₁₋₆alkoxycarbonyloxyethyl esters for example 1-methoxycarbonyloxyethyl and may be formed at any carboxy group in the compounds of this invention.

An in vivo hydrolysable ester of a compound of the formula (I), or other compounds disclosed herein, containing a hydroxy group includes inorganic esters such as phosphate esters and α-acyloxyalkyl ethers and related compounds which as a result of the in vivo hydrolysis of the ester breakdown to give the parent hydroxy group. Examples of α-acyloxyalkyl ethers include acetoxymethoxy and 2,2-dimethylpropionyloxy-methoxy. A selection of in vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl. Examples of substituents on benzoyl include morpholino and piperazino linked from a ring nitrogen atom via a methylene group to the 3- or 4-position of the benzoyl ring.

A suitable value for an in vivo hydrolysable amide of a compound of the formula (I), or other compounds disclosed herein, containing a carboxy group is, for example, a N-C₁₋₆alkyl or N,N-di-C₁₋₆alkyl amide such as N-methyl, N-ethyl, N-propyl, N,N-dimethyl, N-ethyl-N-methyl or N,N-diethyl amide.

Some compounds of the formula (I) may have chiral centres and/or geometric isomeric centres (E- and Z-isomers), and it is to be understood that the invention encompasses all such optical, diastereoisomers and geometric isomers that possess cholesterol absorption inhibitory activity.

The invention relates to any and all tautomeric forms of the compounds of the formula (I) that possess cholesterol absorption inhibitory activity.

It is also to be understood that certain compounds of the formula (I) can exist in solvated as well as unsolvated forms such as, for example, hydrated forms. It is to be understood that the invention encompasses all such solvated forms which possess cholesterol absorption inhibitory activity.

Preferred aspects of the invention are those which relate to the compound of formula (I) or a pharmaceutically acceptable salt thereof.

Another aspect of the present invention provides a process for preparing a compound of formula (I) or a pharmaceutically acceptable salt, solvate, solvate of such a salt or a prodrug thereof which process (wherein variable groups are, unless otherwise specified, as defined in formula (I)) comprises of:

### Process 1) Reacting a Compound of Formula (II):

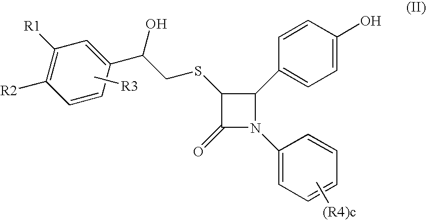

with a compound of formula (III):

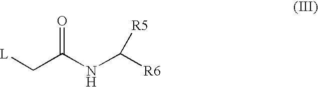

wherein L is a displaceable group;

### Process 2) Reacting an Acid of Formula (IV):

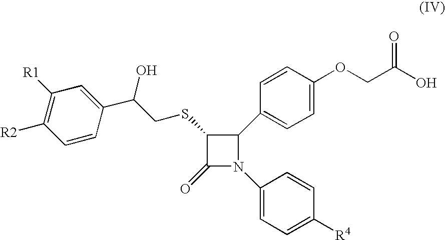

or an activated derivative thereof; with an amine of formula (V):

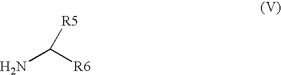

### Process 3): Reducing a Compound of Formula (VI):

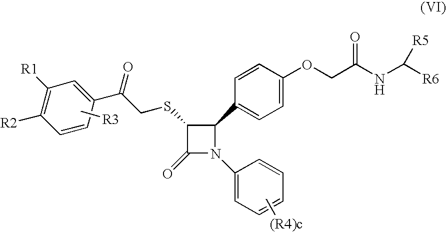

### Process 4): Reacting a Compound of Formula (VII):

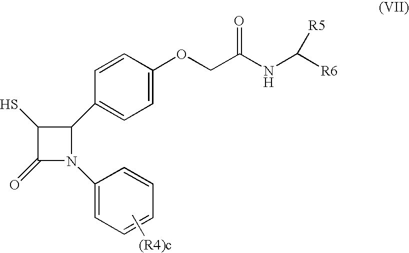

with a compound of formula (VIII):

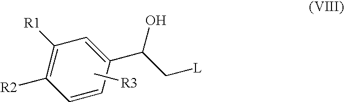

wherein L is a displaceable group;

### Process 5): Reacting a Compound of Formula (IX):

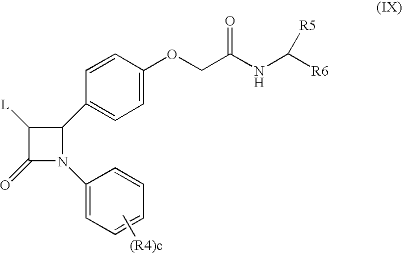

wherein L is a displaceable group; with a compound of formula (X):

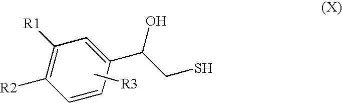

and thereafter if necessary or desirable:
i) converting a compound of the formula (I) into another compound of the formula (I);
ii) removing any protecting groups;
iii) forming a pharmaceutically acceptable salt, solvate, solvate of such a salt or a prodrug; or
iv) separating two or more enantiomers.

L is a displaceable group, suitable values for L are for example, a halogeno or sulphonyloxy group, for example a chloro, bromo, methanesulphonyloxy or toluene-4-sulphonyloxy group.

C(O)OR is an ester group, suitable values for C(O)OR are methoxycarbonyl, ethoxycarbonyl, t-butoxycarbonyl and benzyloxycarbonyl.

The starting materials used in the present invention can be prepared by modifications of the routes described in EP 0 792 264 B1. Alternatively they can be prepared by the following reactions.

Process 1): Alcohols of formula (II) may be reacted with compounds of formula (III) in the presence of a base for example an inorganic base such as sodium carbonate, or an organic base such as Hunigs base, in the presence of a suitable solvent such as acetonitrile, dichloromethane or tetrahydrofuran at a temperature in the range of 0° C. to reflux, preferably at or near reflux.

Compounds of formula (II) may be prepared according to the following scheme:

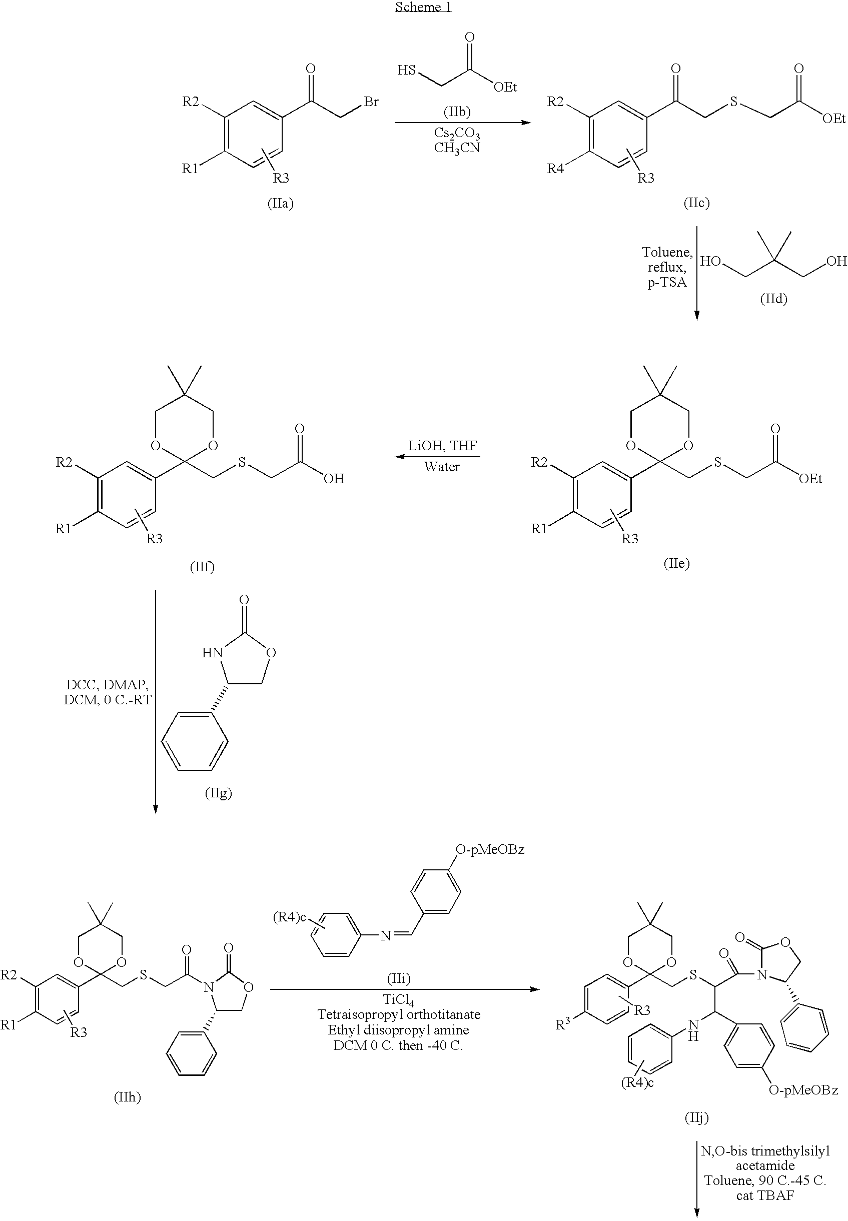

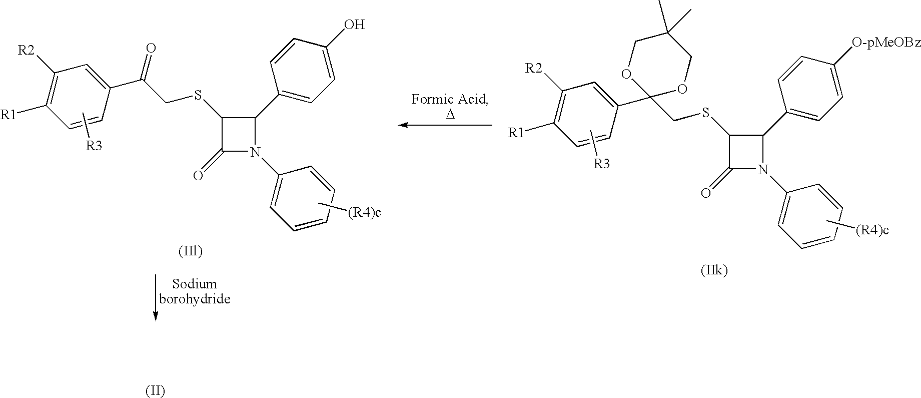

wherein pMeOBz is para methoxy benzyl.

Compounds of formula (IIb), (IId), (IIg) and (III) are commercially available compounds, or they are known in the literature, or they are prepared by standard processes known in the art.

Another aspect of the present invention provides a process for preparing a compound of formula (I2) or a pharmaceutically acceptable salt, solvate, solvate of such a salt or a prodrug thereof which process (wherein variable groups are, unless otherwise specified, as defined in formula (I)) comprises of:

### Process 1) Reacting a Compound of Formula (II2):

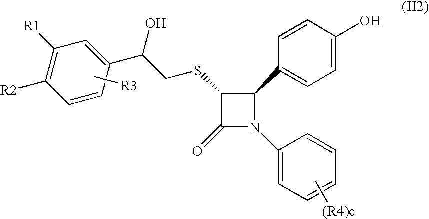

with a compound of formula (III):

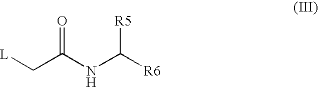

wherein L is a displaceable group;

### Process 2) Reacting an Acid of Formula (IV2):

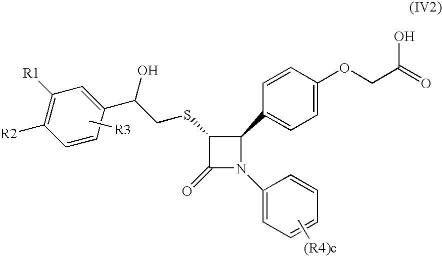

or an activated derivative thereof; with an amine of formula (V):

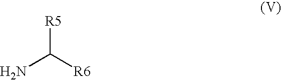

### Process 3): Reducing a Compound of Formula (VI2):

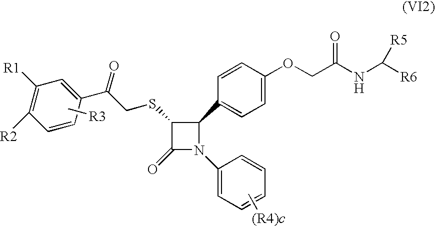

### Process 4): Reacting a Compound of Formula (VII2):

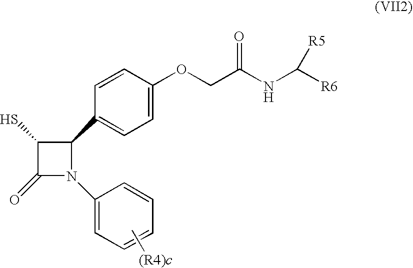

with a compound of formula (VIII):

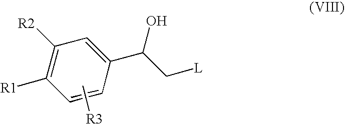

wherein L is a displaceable group;

### Process 5): Reacting a Compound of Formula (IX2):

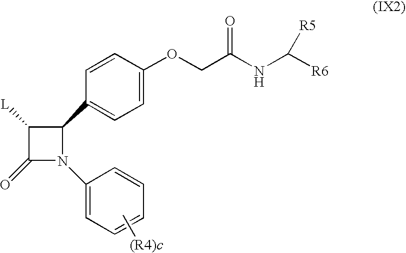

wherein L is a displaceable group; with a compound of formula (X):

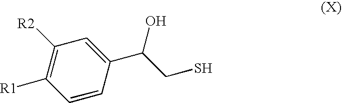

and thereafter if necessary or desirable:
i) converting a compound of the formula (I2) into another compound of the formula (I2);
ii) removing any protecting groups;
iii) forming a pharmaceutically acceptable salt, solvate, solvate of such a salt or a prodrug; or
iv) separating two or more enantiomers.

L is a displaceable group, suitable values for L are for example, a halogeno or sulphonyloxy group, for example a chloro, bromo, methanesulphonyloxy or toluene-4-sulphonyloxy group.

C(O)OR is an ester group, suitable values for C(O)OR are methoxycarbonyl, ethoxycarbonyl, t-butoxycarbonyl and benzyloxycarbonyl.

The starting materials used in the present invention can be prepared by modifications of the routes described in EP 0 792 264 B1. Alternatively they can be prepared by the following reactions.

Process 1): Alcohols of formula (II2) may be reacted with compounds of formula (III) in the presence of a base for example an inorganic base such as sodium carbonate, or an organic base such as Hunigs base, in the presence of a suitable solvent such as acetonitrile, dichloromethane or tetrahydrofuran at a temperature in the range of 0° C. to reflux, preferably at or near reflux.

Compounds of formula (II2) may be prepared according to the following scheme:

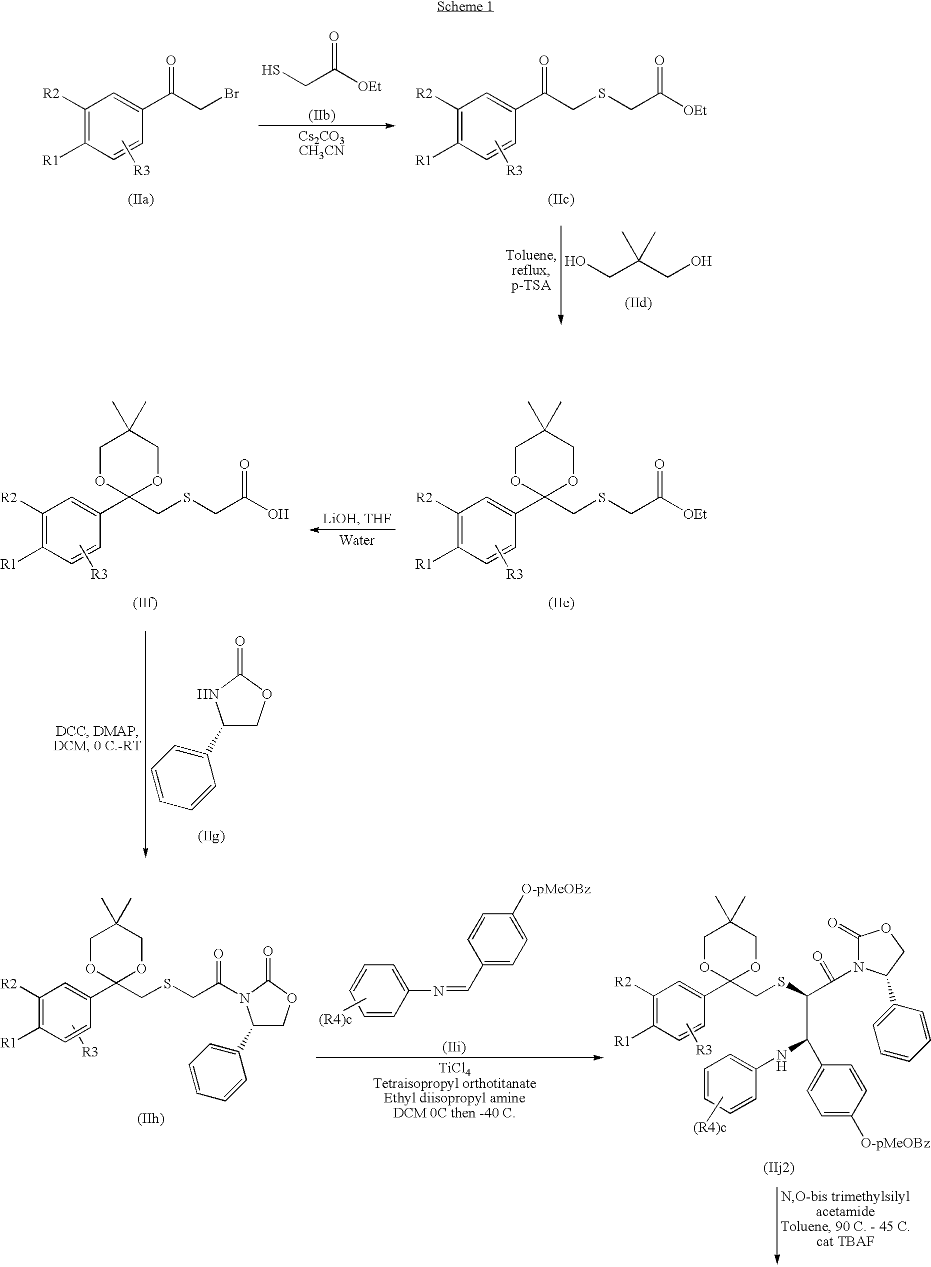

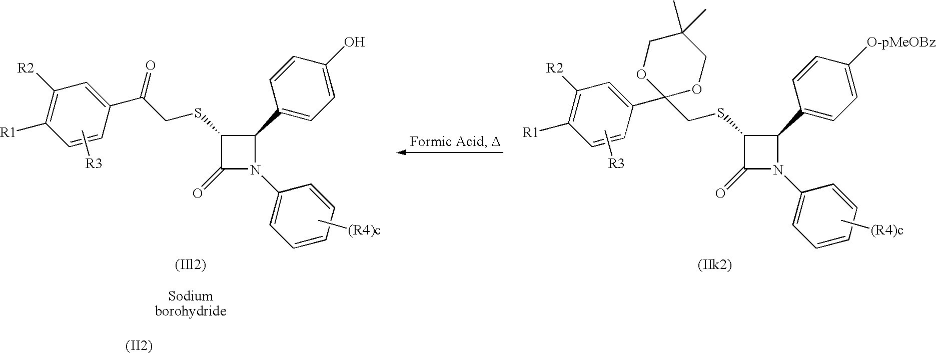

wherein pMeOBz is para methoxy benzyl.

Compounds of formula (Ib), (IId), (IIg) and (IIi) are commercially available compounds, or they are known in the literature, or they are prepared by standard processes known in the art.

A compound of formula (V) may also be reacted with a compound of formula (XI).

Compounds of formula (XI) may be prepared according to the following route:

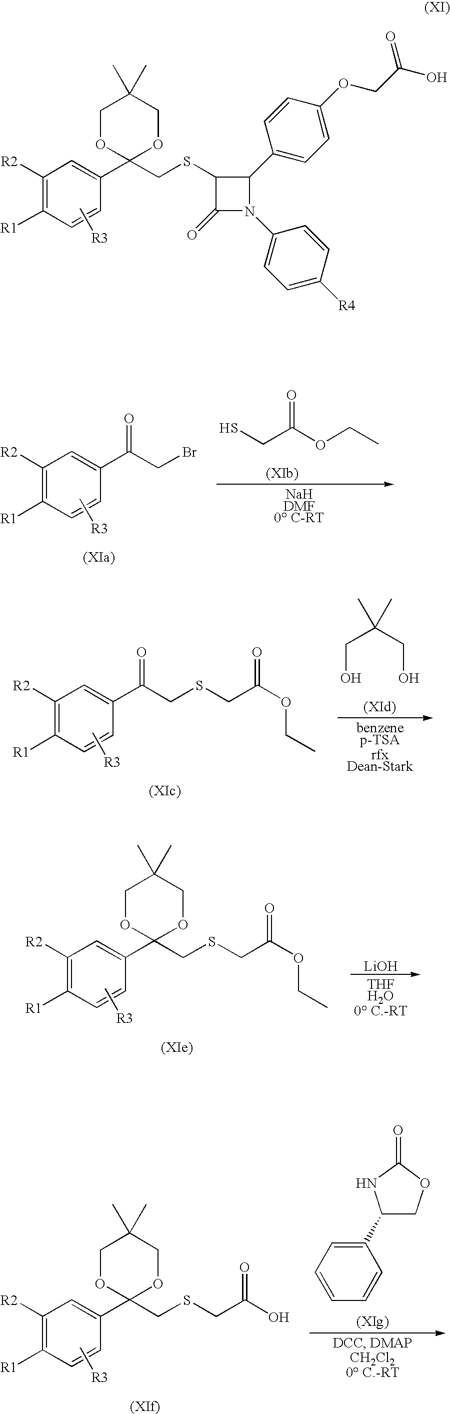

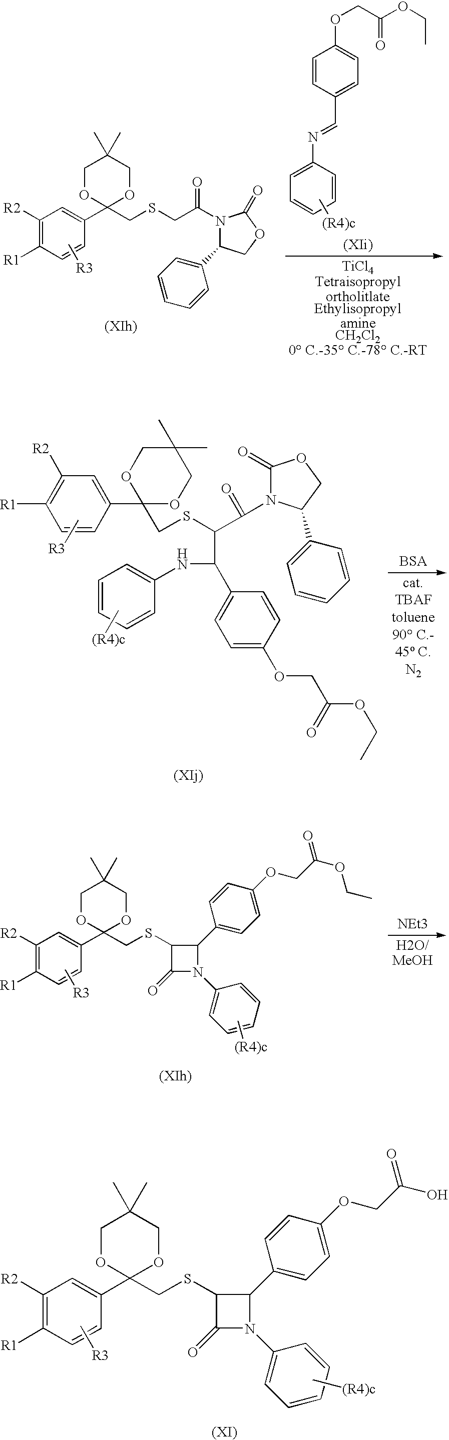

Compounds of formula XII may be prepared by the following route:

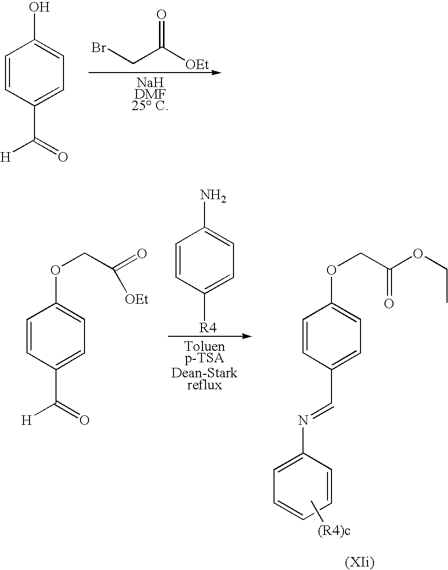

A compound of formula (V) may also be reacted with a compound of formula (XI2).

Compounds of formula (XI2) may be prepared according to the following route:

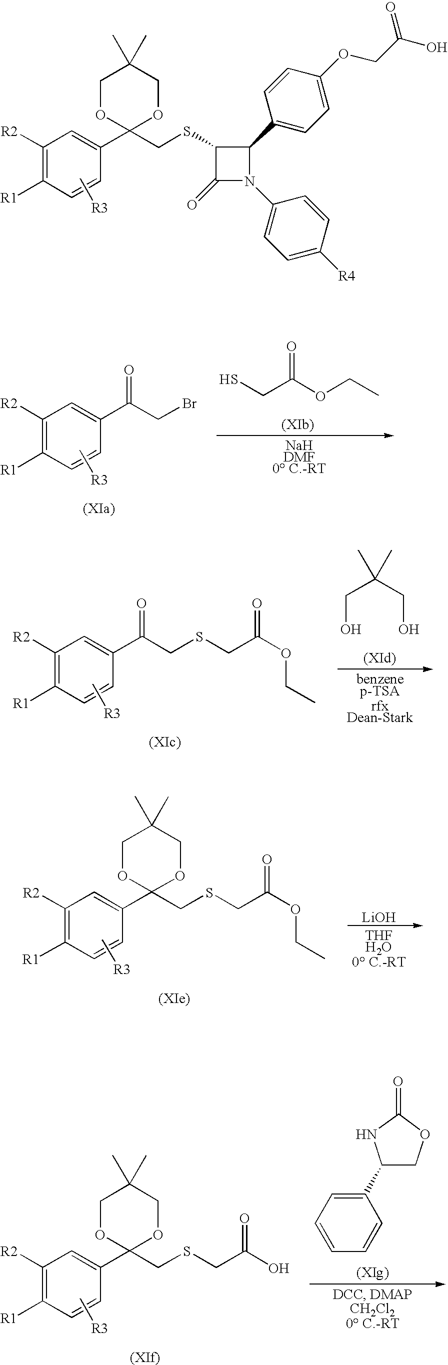

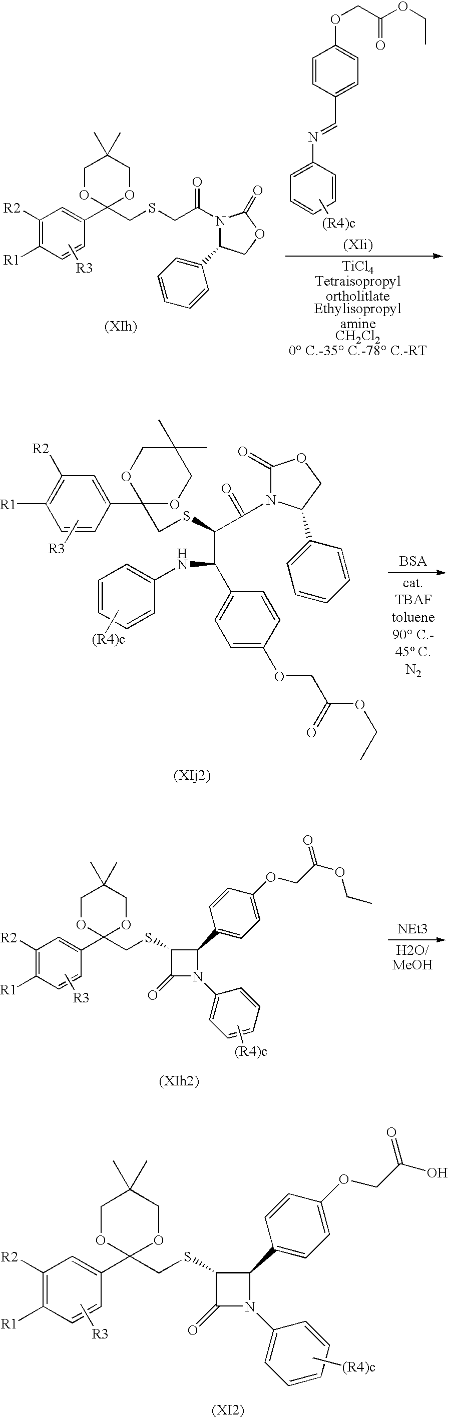

Compounds of formula XII may be prepared by the following route:

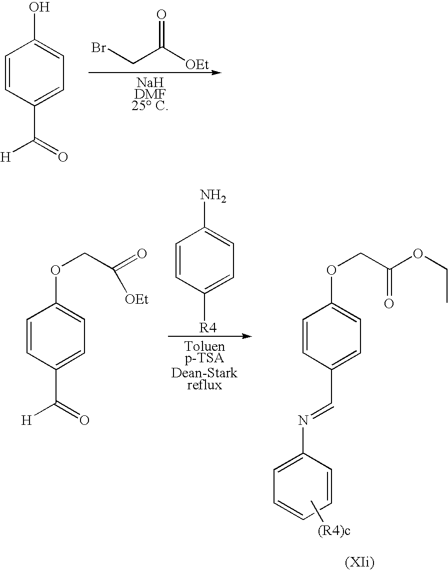

For XI and XI2 both, the following applies:

Process 2): Acids and amines may be coupled together in the presence of a suitable coupling reagent. Standard peptide coupling reagents known in the art can be employed as suitable coupling reagents, for example carbonyldiimidazole and dicyclohexyl-carbodiimide, optionally in the presence of a catalyst such as dimethylaminopyridine or 4-pyrrolidinopyridine, optionally in the presence of a base for example triethylamine, pyridine, or 2,6-di-alkyl-pyridines such as 2,6-lutidine or 2,6-di-tert-butylpyridine. Suitable solvents include dimethylacetamide, dichloromethane, benzene, tetrahydrofuran and dimethylformamide. The coupling reaction may conveniently be performed at a temperature in the range of −40 to 40° C.

Suitable activated acid derivatives include acid halides, for example acid chlorides, and active esters, for example pentafluorophenyl esters. The reaction of these types of compounds with amines is well known in the art, for example they may be reacted in the presence of a base, such as those described above, and in a suitable solvent, such as those described above. The reaction may conveniently be performed at a temperature in the range of −40 to 40° C.

Acids of formula (IV) may be prepared from compounds of formula (II) by reacting them with the appropriate, optionally protected, side chain using the conditions of Process 1). Alternatively, acids of formula (IV) may be prepared by a modification of Scheme I.

Amines of formula (V) are commercially available compounds, or they are known in the literature, or they are prepared by standard processes known in the art.

Process 4): Reduction of compounds of formula (VI) could be performed with a hydride reagent such as sodium borohydride in a solvent such as methanol at temperatures suitable between −20-40° C.

Compounds of formula (VI) can be prepared from compounds of formula (III), by deprotecting the benzyl group and performing Process 1. Alternatively compound (IIk) could be debenzylated, Process 1 could be performed and the resulting compound deprotected to reveal the ketone.

Process 4) and Process 5): these compounds may be reacted together in the presence of a base for example an inorganic base such as sodium carbonate, or an organic base such as Hunigs base, in the presence of a suitable solvent such as acetonitrile, dichloromethane or tetrahydrofuran at a temperature in the range of 0° C. to reflux, preferably at or near reflux. Compounds of formula (VII) and (IX) may be prepared by an appropriate modification of Scheme I.

Compounds of formula (VIII) and (X) are commercially available compounds, or they are known in the literature, or they are prepared by standard processes known in the art.

Process 7): Esters of formula (XIII) may be deprotected under standard conditions such as those described below, for example a methyl or ethyl ester may be deprotected with sodium hydroxide in methanol at room temperature.

Compounds of formula (XI) may be prepared by a modification of any of the processes described herein for the preparation of compounds of formula (I).

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogeno group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulphinyl or alkylsulphonyl.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1999). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

As stated hereinbefore the compounds defined in the present invention possess cholesterol absorption inhibitory activity. These properties may be assessed, using the following biological tests.

### In Vivo Testing of Cholesterol Absorption Inhibitors (A)

C57BL/6 female mice were maintained on regular chow diet and housed in individual cages to collect faeces. Mice were fasted for 3 hours and then gavaged with vehicle or compound. Half an hour later the mice were gavaged with radiolabelled cholesterol. Six hours after the ¹⁴C-cholesterol gavage blood samples were taken via the tail and plasma prepared to determine how much cholesterol were absorbed. 24 hours after the gavage of ¹⁴C-cholesterol the mice were bled and plasma were prepared for analysis. Faeces were collected for 24 hours to assess absorption efficiency.

### In Vivo Testing of Cholesterol Absorption Inhibitors (B).

C57BL/6 female mice were maintained on regular chow diet and housed in individual cages to collect faeces. Mice were fasted for 3 hours and then gavaged with vehicle or compound. One to ten hours later the mice were gavaged with radiolabelled cholesterol. Six hours after the ¹⁴C-cholesterol gavage blood sample was taken via the tail and plasma prepared to determine how much cholesterol was absorbed. 24 hours after the gavage of ¹⁴C-cholesterol the mice were bled and plasma analysed for radioactivity. Faeces were also collected for 24 hours to assess absorption efficiency.

### REFERENCES

1. E. A. Kirk, G. L. Moe, M. T. Caldwell, J. Å. Lerrunark, D. L. Wilson, R. C. LeBoeuf. Hyper- and hypo-responsiveness to dietary fat and cholesterol among inbred mice: searching for level and variability genes. J. Lipid Res. 1995 36:1522-1532.
2. C. P. Carter, P. N. Howles, D. Y. Hui. Genetic variation in cholesterol absorption efficiency among inbred strains of mice. J. Nutr. 1997 127:1344-1348.
3. C. D. Jolley, J. M. Dietschy, S. D. Turley. Genetic differences in cholesterol absorption in 129/Sv and C57BL/6 mice: effect on cholesterol responsiveness. Am. J. Physiol. 1999 276:G1117-G1124.

Administration of 0.2 μmol/kg of Example 10 gave 38% inhibition of ¹⁴C-cholesterol absorption (procedure A).

According to a further aspect of the invention there is provided a pharmaceutical composition which comprises a compound of formula (I), or a pharmaceutically acceptable salt, solvate, solvate of such a salt or a prodrug thereof, as defined hereinbefore in association with a pharmaceutically-acceptable diluent or carrier.

The composition may be in a form suitable for oral administration, for example as a tablet or capsule, for parenteral injection (including intravenous, subcutaneous, intramuscular, intravascular or infusion) as a sterile solution, suspension or emulsion, for topical administration as an ointment or cream or for rectal administration as a suppository.

In general the above compositions may be prepared in a conventional manner using conventional excipients.

The compound of formula (I), or a pharmaceutically acceptable salt, solvate, solvate of such a salt or a prodrug thereof, will normally be administered to a warm-blooded animal at a unit dose within the range of approximately 0.02-100 mg/kg, preferably 0.02-50 mg/kg, and this normally provides a therapeutically-effective dose. A unit dose form such as a tablet or capsule will usually contain, for example 1-250 mg of active ingredient. Preferably a daily dose in the range of 1-50 mg/kg, particularly 0.1-10 mg/kg is employed. In another aspect a daily dose in the rage of 0.01-20 mg/kg is employed. In one aspect of the invention the daily dose of a compound of formula (I) is less than or equal to 100 mg. However the daily dose will necessarily be varied depending upon the host treated, the particular route of administration, and the severity of the illness being treated. Accordingly the optimum dosage may be determined by the practitioner who is treating any particular patient.

According to a further aspect of the present invention there is provided a compound of the formula (I), or a pharmaceutically acceptable salt, solvate, solvate of such a salt or a prodrug thereof, as defined hereinbefore for use in a method of prophylactic or therapeutic treatment of a warm-blooded animal, such as man.

We have found that the compounds defined in the present invention, or a pharmaceutically acceptable salt, solvate, solvate of such a salt or a prodrug thereof, are effective cholesterol absorption inhibitors, and accordingly have value in the treatment of disease states associated with hyperlipidaemic conditions.

Thus according to this aspect of the invention there is provided a compound of the formula (I), or a pharmaceutically acceptable salt, solvate, solvate of such a salt or a prodrug thereof, as defined hereinbefore for use as a medicament.

According to another feature of the invention there is provided the use of a compound of the formula (I), or a pharmaceutically acceptable salt, solvate, solvate of such a salt or a prodrug thereof, as defined hereinbefore in the manufacture of a medicament for use in the production of a cholesterol absorption inhibitory effect in a warm-blooded animal, such as man.

According to another feature of the invention there is provided the use of a compound of the formula (I), or a pharmaceutically acceptable salt, solvate, solvate of such a salt or a prodrug thereof, as defined hereinbefore in the production of a cholesterol absorption inhibitory effect in a warm-blooded animal, such as man.

Herein, where the production of a cholesterol absorption inhibitory effect or a cholesterol lowering effect is stated, suitably this relates to the treatment of hyperlipidaemic conditions in a warm-blooded animal, such as man. Additionally is relates to the treatment of dyslipidemic conditions and disorders such as hyperlipidaemia, hypertrigliceridemia, hyperbetalipoproteinemia (high LDL), hyperprebetalipoproteinemia (high VLDL), hyperchylomicronemia, hypolipoproteineraia, hypercholesterolemia, hyperlipoproteinemia and hypoalphalipoproteinemia (low HDL) in a warm-blooded animal, such as man. Furthermore it relates to the treatment of different clinical conditions such as atherosclerosis, arteriosclerosis, arrhythmia, hyper-thrombotic conditions, vascular dysfunction, endothelial dysfunction, heart failure, coronary heart diseases, cardiovascular diseases, myocardial infarction, angina pectoris, peripheral vascular diseases, inflammation of cardiovascular tissues such as heart, valves, vasculature, arteries and veins, aneurisms, stenosis, restenosis, vascular plaques, vascular fatty streaks, leukocytes, monocytes and/or macrophage infiltration, intimal thickening, medial thinning, infectious and surgical trauma and vascular thrombosis, stroke and transient ischaemic attacks in a warm-blooded animal, such as man. It also relates to the treatment of atherosclerosis, coronary heart diseases, myocardial infarction, angina pectoris, peripheral vascular diseases, stroke and transient ischaemic attacks in a warm-blooded animal, such as man.

The production of a cholesterol absorption inhibitory effect or a cholesterol lowering effect also relates to a method of treating and/or preventing atherosclerotic lesions, a method of preventing plaque rupture and a method of promoting lesion regression. Furthermore it relates to a method of inhibiting monocytes-macrophage accumulation in atherosclerotic lesions, a method of inhibiting expression of matrix metalloproteinases in atherosclerotic lesions, a method of inhibiting the destabilization of atherosclerotic lesions, a method for preventing atherosclerotic plaque rupture and a method of treating unstable angina.

The production of a cholesterol absorption inhibitory effect or a cholesterol lowering effect also relates to a method of treating sitosterolemia.

Compounds of formula (I), or a pharmaceutically acceptable salt, solvate, solvate of such a salt or a prodrug thereof may also have value in the treatment or prevention of Alzheimer's Disease (see for example WO 02/096415). Therefore in a further aspect of the invention, there is provided a compound of formula (I), or a pharmaceutically acceptable salt, solvate, solvate of such a salt or a prodrug thereof, for use in the treatment or prevention of Alzheimer's Disease.

Compounds of formula (I), or a pharmaceutically acceptable salt, solvate, solvate of such a salt or a prodrug thereof may also have value in the treatment or prevention of cholesterol associated tumors. Therefore in a further aspect of the invention, there is provided a compound of formula (I), or a pharmaceutically acceptable salt, solvate, solvate of such a salt or a prodrug thereof, for use in the treatment or prevention of cholesterol associated tumors.

Compounds of formula (I), or a pharmaceutically acceptable salt, solvate, solvate of such a salt or a prodrug thereof may also have value in the treatment or prevention of vascular inflammation (see for example WO 03/026644). Therefore in a further aspect of the invention, there is provided a compound of formula (I), or a pharmaceutically acceptable salt, solvate, solvate of such a salt or a prodrug thereof, for use in the treatment or prevention of vascular inflammation.

According to a further feature of this aspect of the invention there is provided a method for producing a cholesterol absorption inhibitory effect in a warm-blooded animal, such as man, in need of such treatment which comprises administering to said animal an effective amount of a compound of formula (I), or a pharmaceutically acceptable salt, solvate, solvate of such a salt or a prodrug thereof.

The cholesterol absorption inhibitory activity defined hereinbefore may be applied as a sole therapy or may involve, in addition to a compound of the invention, one or more other substances and/or treatments. Such conjoint treatment may be achieved by way of the simultaneous, sequential or separate administration of the individual components of the treatment. According to this aspect of the invention there is provided a pharmaceutical product comprising a compound of the formula (I), or a pharmaceutically acceptable salt, solvate, solvate of such a salt or a prodrug thereof, as defined hereinbefore and an additional cholesterol absorption inhibitory substance as defined hereinbefore and an additional hypolipidaemic agent for the conjoint treatment of hyperlipidaemia.

In another aspect of the invention, the compound of formula (I), or a pharmaceutically acceptable salt, solvate, solvate of such a salt or a prodrug thereof, may be administered in association with cholesterol biosynthesis inhibitors, or pharmaceutically acceptable salts, solvates, solvates of such salts or prodrugs thereof. Suitable cholesterol biosynthesis inhibitors include HMG Co-A reductase inhibitors, squalene synthesis inhibitors and squalene epoxidase inhibitors. Suitable squalene synthesis inhibitors are e.g squalestatin 1, TAK 475 and compounds described in WO2005012284. A suitable squalene epoxidase inhibitor is NB-598.

In this aspect of the invention, the compound of formula (I), or a pharmaceutically acceptable salt, solvate, solvate of such a salt or a prodrug thereof, may be administered in association with an HMG Co-A reductase inhibitor, or pharmaceutically acceptable salts, solvates, solvates of such salts or prodrugs thereof. Suitable HMG Co-A reductase inhibitors, pharmaceutically acceptable salts, solvates, solvates of such salts or prodrugs thereof are statins well known in the art. Particular statins are fluvastatin, lovastatin, pravastatin, simvastatin, atorvastatin, cerivastatin, bervastatin, dalvastatin, mevastatin and rosuvastatin, or a pharmaceutically acceptable salt, solvate, solvate of such a salt or a prodrug thereof. A further particular statin is pitavastatin, or a pharmaceutically acceptable salt, solvate, solvate of such a salt or a prodrug thereof. A particular statin is atorvastatin, or a pharmaceutically acceptable salt, solvate, solvate of such a salt or a prodrug thereof. A more particular statin is atorvastatin calcium salt. A further particular statin is rosuvastatin, or a pharmaceutically acceptable salt, solvate, solvate of such a salt or a prodrug thereof. A preferable particular statin is rosuvastatin calcium salt.

Therefore in an additional feature of the invention, there is provided a combination of a compound of formula (I), or a pharmaceutically acceptable salt, solvate, solvate of such a salt or a prodrug thereof and an HMG Co-A reductase inhibitor, or a pharmaceutically acceptable salt, solvate, solvate of such a salt or a prodrug thereof.

Therefore in an additional feature of the invention, there is provided a method for producing a cholesterol lowering effect in a warm-blooded animal, such as man, in need of such treatment which comprises administering to said animal an effective amount of a compound of formula (I), or a pharmaceutically acceptable salt, solvate, solvate of such a salt or a prodrug thereof in simultaneous, sequential or separate administration with an effective amount of an HMG Co-A reductase inhibitor, or a pharmaceutically acceptable salt, solvate, solvate of such a salt or a prodrug thereof.

According to a further aspect of the invention there is provided a pharmaceutical composition which comprises a compound of formula (I), or a pharmaceutically acceptable salt, solvate, solvate of such a salt or a prodrug thereof, and an HMG Co-A reductase inhibitor, or a pharmaceutically acceptable salt, solvate, solvate of such a salt or a prodrug thereof, in association with a pharmaceutically acceptable diluent or carrier.

According to a further aspect of the present invention there is provided a kit comprising a compound of formula (I), or a pharmaceutically acceptable salt, solvate, solvate of such a salt or a prodrug thereof, and an HMG Co-A reductase inhibitor, or a pharmaceutically acceptable salt, solvate, solvate of such a salt or a prodrug thereof.

According to a further aspect of the present invention there is provided a kit comprising:

a) a compound of formula (I), or a pharmaceutically acceptable salt, solvate, solvate of such a salt or a prodrug thereof, in a first unit dosage form;
b) an HMG Co-A reductase inhibitor, or a pharmaceutically acceptable salt, solvate; solvate of such a salt or a prodrug thereof; in a second unit dosage form; and
c) container means for containing said first and second dosage forms.

According to a further aspect of the present invention there is provided a kit comprising:

a) a compound of formula (I), or a pharmaceutically acceptable salt, solvate, solvate of such a salt or a prodrug thereof, together with a pharmaceutically acceptable diluent or carrier, in a first unit dosage form;
b) an HMG Co-A reductase inhibitor, or a pharmaceutically acceptable salt, solvate, solvate of such a salt or a prodrug thereof, in a second unit dosage form; and
c) container means for containing said first and second dosage forms.

According to another feature of the invention there is provided the use of a compound of the formula (I), or a pharmaceutically acceptable salt, solvate, solvate of such a salt or a prodrug thereof, and an HMG Co-A reductase inhibitor, or a pharmaceutically acceptable salt, solvate, solvate of such a salt or a prodrug thereof, in the manufacture of a medicament for use in the production of a cholesterol lowering effect.

According to a further aspect of the present invention there is provided a combination treatment comprising the administration of an effective amount of a compound of the formula (I), or a pharmaceutically acceptable salt, solvate, solvate of such a salt or a prodrug thereof, optionally together with a pharmaceutically acceptable diluent or carrier, with the simultaneous, sequential or separate administration of an effective amount of an HMG Co-A reductase inhibitor, or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

According to an additional further aspect of the present invention there is provided a combination treatment comprising the administration of an effective amount of a compound of the formula (I), or a pharmaceutically acceptable salt, solvate, solvate of such a salt or a prodrug thereof, optionally together with a pharmaceutically acceptable diluent or carrier, with the simultaneous, sequential or separate administration of a matrix metalloproteinase inhibitor.

In another aspect of the invention, the compound of formula (I), or a pharmaceutically acceptable salt, solvate, solvate of such a salt or a prodrug thereof, may be administered in association with an ileal bile acid (THAT) inhibitor or a pharmaceutically acceptable salt, solvate, solvate of such a salt or a prodrug thereof. Suitable compounds possessing IBAT inhibitory activity for use in combination with compounds of the present invention have been described, see for instance the compounds described in WO 93/16055, WO 94/18183, WO 94/18184, WO 94/24087, WO 96/05188, WO 96/08484, WO 96/16051, WO 97/33882, WO 98/07749, WO 98/38182, WO 98/40375, WO 98/56757, WO 99/32478, WO 99/35135, WO 99/64409, WO 99/64410, WO 00/01687, WO 00/20392, WO 00/20393, WO 00/20410, WO 00/20437, WO 00/35889, WO 01/34570, WO 00/38725, WO 00/38726, WO 00/38727, WO 00/38728, WO 00/38729, WO 00/47568, WO 00/61568, WO 01/66533, WO 01/68096, WO 01/68637, WO 02/08211, WO 02/50051, WO 03/018024, WO 03/040127, WO 03/043992, WO 03/061604, WO 04/020421, WO 04/076430, DE 19825804, JP 10072371, U.S. Pat. No. 5,070,103, EP 251 315, EP 417 725, EP 489 423, EP 549 967, EP 573 848, EP 624 593, EP 624 594, EP 624 595, EP 864 582, EP 869 121 and EP 1 070 703, WO 03/020710, WO 03/022825, WO 03/022830, WO 03/022286, WO 03/091232, WO 03/106482 and EP 597 107.

and the contents of these patent applications are incorporated herein by reference. Particularly the named examples of these patent applications are incorporated herein by reference. More particularly claim 1 of these patent application are incorporated herein by reference.

Other suitable classes of IBAT inhibitors for use in combination with compounds of the present invention are the benzothiepines, 1,2-benzothiazepines, 1,4-benzothiazepines and 1,5-benzothiazepines. A further suitable class of IBAT inhibitors is the 1,2,5-benzothiadiazepines.

One particular suitable compound possessing IBAT inhibitory activity for use in combination with compounds of the present invention is (3R,5R)-3-butyl-3-ethyl-1,1-dioxido-5-phenyl-2,3,4,5-tetrahydro-1,4-benzothiazepin-8-yl beta-D-glucopyranosiduronic acid (EP 864 582).

A further suitable compound possessing IBAT inhibitory activity for use in combination with compounds of the present invention is S-8921 (EP 597 107) and BARI-1741.

A further suitable IBAT inhibitor for use in combination with compounds of the present invention is the compound:

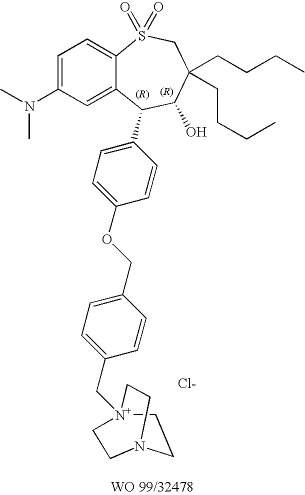

A particular IBAT inhibitor for use in combination with compounds of the present invention is selected from any one of Examples 1-120 of WO 02/50051, or a pharmaceutically acceptable salt, solvate, solvate of such a salt or a prodrug thereof, and the compounds of Examples 1-120 are incorporated herein by reference. Claims 1-15 of WO 02/50051 are also incorporated herein by reference. A particular IBAT inhibitor selected from WO 02/50051 for use in combination with compounds of the present invention is selected from any one of:
1,1-dioxo-3,3-dibutyl-5-phenyl-7-methylthio-8-(N-{(R)-1′-phenyl-1′-[N′-(carboxymethyl)carbamoyl]methyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-(carboxymethyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N-{(R)-1′-phenyl-1′-[N′-(2-sulphoethyl)carbamoyl]methyl}carbamoylmethoxy)-2,3,4,5-tetrahydro-1,5-benzothiazepine;
1,1-dioxo-3-butyl-3-ethyl-5-phenyl-7-methylthio-8-(N-{(R)-1′-phenyl-1′-[N′-(2-sulphoethyl)carbamoyl]methyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-(2-sulphoethyl)carbamoyl]-4-hydroxybenzyl}carbamoylmethoxy)-2,3,4,5-tetrahydro-1,5-benzothiazepine;
1,1-dioxo-3-butyl-3-ethyl-5-phenyl-7-methylthio-8-(N-{(R)-α-[N′-(2-sulphoethyl)carbamoyl]-4-hydroxybenzyl}carbamoylmethoxy)-2,3,4,5-tetrahydro-1,5-benzothiazepine;
1,1-dioxo-3-butyl-3-ethyl-5-phenyl-7-methylthio-8-(N-{(R)-α-[N′-(2-carboxyethyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-(2-carboxyethyl)carbamoyl]-4-hydroxybenzyl}carbamoylmethoxy)-2,3,4,5-tetrahydro-1,5-benzothiazepine;
1,1-dioxo-3-butyl-3-ethyl-5-phenyl-7-methylthio-8-(N-{(R)-α-[N′-(5-carboxypentyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-(2-carboxyethyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N-{α-[N′-(2-sulphoethyl)carbamoyl]-2-fluorobenzyl}carbamoylmethoxy)-2,3,4,5-tetrahydro-1,5-benzothiazepine;
1,1-dioxo-3-butyl-3-ethyl-5-phenyl-7-methylthio-8-(N-{(R)-α-[N′-(R)-(2-hydroxy-1-carboxyethyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-(R)-(2-hydroxy-1-carboxyethyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-{N′-[(R)-α-(N′-{(R)-1-[N″-(R)-(2-hydroxy-1-carboxyethyl)carbamoyl]-2-hydroxyethyl}carbamoyl)benzyl]carbamoylmethoxy}-2,3,4,5-tetrahydro-1,5-benzothiazepine;
1,1-dioxo-3-butyl-3-ethyl-5-phenyl-7-methylthio-8-(N-{α-[N′-(carboxymethyl)carbamoyl]benzyl}carbamoylmethoxy)-2,3,4,5-tetrahydro-1,5-benzothiazepine;
1,1-dioxo-3-butyl-3-ethyl-5-phenyl-7-methylthio-8-(N-{α-[N′-((ethoxy)(methyl)phosphoryl-methyl)carbamoyl]benzyl}carbamoylmethoxy)-2,3,4,5-tetrahydro-1,5-benzothiazepine;
1,1-dioxo-3-butyl-3-ethyl-5-phenyl-7-methylthio-8-{N-[(R)-α-(N′-{2-[(hydroxy)(methyl)phosphoryl]ethyl}carbamoyl)benzyl]carbamoylmethoxy}-2,3,4,5-tetrahydro-1,5-benzothiazepine;
1,1-dioxo-3,3-dibutyl-5-phenyl-7-methylthio-8-(N-{(R)-α-[N′-(2-methylthio-1-carboxyethyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-{N-[(R)-α-(N′-{2-[(methyl)(ethyl)phosphoryl]ethyl}carbamoyl)-4-hydroxybenzyl]carbamoylmethoxy}-2,3,4,5-tetrahydro-1,5-benzothiazepine;
1,1-dioxo-3,3-dibutyl-5-phenyl-7-methylthio-8-{N-[(R)-α-(N′-{2-[(methyl)(hydroxy)phosphoryl]ethyl}carbamoyl)-4-hydroxybenzyl]carbamoylmethoxy}-2,3,4,5-tetrahydro-1,5-benzothiazepine;
1,1-dioxo-3,3-dibutyl-5-phenyl-7-methylthio-8-(N-{(R)-α-[(R)-N′-(2-methylsulphinyl-1-carboxyethyl)carbamoyl]benzyl}carbamoylmethoxy)-2,3,4,5-tetrahydro-1,5-benzothiazepine; and
1,1-dioxo-3,3-dibutyl-5-phenyl-7-methoxy-8-[N-{(R)-α-[N′-(2-sulphoethyl)carbamoyl]-4-hydroxybenzyl}carbamoylmethoxy]-2,3,4,5-tetrahydro-1,5-benzothiazepine;
   or a pharmaceutically acceptable salt, solvate, solvate of such a salt or a prodrug thereof.

A particular IBAT inhibitor for use in combination with compounds of the present invention is selected from any one of Examples 1-44 of WO 03/020710, or a pharmaceutically acceptable salt, solvate, solvate of such a salt or a prodrug thereof, and the compounds of Examples 1-44 are incorporated herein by reference. Claims 1-10 of WO 03/020710 are also incorporated herein by reference. A particular MAT inhibitor selected from WO 03/020710 for use in combination with compounds of the present invention is selected from any one of:
1,1-dioxo-3,3-dibutyl-5-phenyl-7-methylthio-8-(N-{(R)-α-[N′-(2-(S)-3-(R)-4-(R)-5-(R)-2,3,4,5,6-pentahydroxyhexyl)carbamoyl]benzyl}carbamoylmethoxy)-2,3,4,5-tetrahydro-1,5-benzothiazepine;
1,1-dioxo-3-butyl-3-ethyl-5-phenyl-7-methylthio-8-(N-{(R)-α-[N′-(2-(S)-3-(R)-4-(R)-5-(R)-2,3,4,5,6-pentahydroxyhexyl)carbamoyl]benzyl}carbamoylmethoxy)-2,3,4,5-tetrahydro-1,5-benzothiazepine;
1,1-dioxo-3-butyl-3-ethyl-5-phenyl-7-methylthio-8-(N-{(R)-α-[N′-((S)-1-carbamoyl-2-hydroxyethyl)carbamoyl]benzyl}carbamoylmethoxy)-2,3,4,5-tetrahydro-1,5-benzothiazepine;
1,1-dioxo-3-butyl-3-ethyl-5-phenyl-7-methylthio-8-(N-{(R)-α-[N-(hydroxycarbamoyl-methyl)carbamoyl]benzyl}carbamoylmethoxy)-2,3,4,5-tetrahydro-1,5-benzothiazepine;
1,1-dioxo-3-butyl-3-ethyl-5-phenyl-7-methylthio-8-[N-((R)-α-{N′-[2-(N′-pyrimidin-2-ylureido)ethyl]carbamoyl}benzyl)carbamoylmethoxy]-2,3,4,5-tetrahydro-1,5-benzothiazepine;
1,1-dioxo-3-butyl-3-ethyl-5-phenyl-7-methylthio-8-[N-((R)-α-{N′-[2-(N′-pyridin-2-ylureido)ethyl]carbamoyl}benzyl)carbamoylmethoxy]-2,3,4,5-tetrahydro-1,5-benzothiazepine;
1,1-dioxo-3-butyl-3-ethyl-5-phenyl-7-methylthio-8-(N-{(R)-α-[N-(1-t-butoxycarbonylpiperidin-4-ylmethyl)carbamoyl]benzyl}carbanaoylmethoxy)-2,3,4,5-tetrahydro-1,5-benzothiazepine;
1,1-dioxo-3-butyl-3-ethyl-5-phenyl-7-methylthio-8-(N-{(R)-α-[N′-(2,3-dihydroxypropyl)carbamoyl]benzyl}carbamoylmethoxy)-2,3,4,5-tetrahydro-1,5-benzothiazepine;
1,1-dioxo-3-butyl-3-ethyl-5-phenyl-7-methylthio-8-[N-((R)-α-{N′-[2-(3,4-dihydroxyphenyl)-2-methoxyethyl]carbamoyl}benzyl)carbamoylmethoxy]-2,3,4,5-tetrahydro-1,5-benzothiazepine
1,1-dioxo-3-butyl-3-ethyl-5-phenyl-7-methylthio-8-(N-{(R)-α-[N′-(2-aminoethyl)carbamoyl]benzyl}carbamoylmethoxy)-2,3,4,5-tetrahydro-1,5-benzothiazepine;
1,1-dioxo-3-butyl-3-ethyl-5-phenyl-7-methylthio-8-(N-{(R)-α-[N′-(piperidin-4-ylmethyl)carbamoyl]benzyl}carbamoylmethoxy)-2,3,4,5-tetrahydro-1,5-benzothiazepine; or
1,1-dioxo-3-butyl-3-ethyl-5-phenyl-7-methylthio-8-(N-{(R)-α-[N′-(2-N,N-dimethylaminosulphamoylethyl)carbamoyl]benzyl}carbamoylmethoxy)-2,3,4,5-tetrahydro-1,5-benzothiazepine;
   or a pharmaceutically acceptable salt, solvate, solvate of such a salt or a prodrug thereof.

A particular IBAT inhibitor for use in combination with compounds of the present invention is selected from any one of Examples 1-7 of WO 03/022825, or a pharmaceutically acceptable salt, solvate, solvate of such a salt or a prodrug thereof, and the compounds of Examples 1-7 are incorporated herein by reference. Claims 1-8 of WO 03/022825 are also incorporated herein by reference. A particular IBAT inhibitor selected from WO 03/022825 for use in combination with compounds of the present invention is selected from any one of:
1,1-dioxo-3(R)-3-butyl-3-ethyl-5-(R)-5-phenyl-8-[N-((R)-α-carboxybenzyl)carbamoylmethoxy]-2,3,4,5-tetrahydro-1,4-benzothiazepine;
1,1-dioxo-3(S)-3-butyl-3-ethyl-5-(S)-5-phenyl-8-[N-((R)-α-carboxybenzyl)carbamoylmethoxy]-2,3,4,5-tetrahydro-1,4-benzothiazepine;
1,1-dioxo-3(R)-3-butyl-3-ethyl-5-(R)-5-phenyl-8-(N-{(R)-α-[N-(carboxymethyl)carbamoyl]benzyl}carbamoylmethoxy)-2,3,4,5-tetrahydro-1,4-benzothiazepine;
1,1-dioxo-3(S)-3-butyl-3-ethyl-5-(S)-5-phenyl-8-(N-{(R)-α-[N-(carboxymethyl)carbamoyl]benzyl}carbamoylmethoxy)-2,3,4,5-tetrahydro-1,4-benzothiazepine;
3,5-trans-1,1-dioxo-3-ethyl-3-butyl-5-phenyl-7-bromo-8-(N-{(R)-α-[N-(carboxymethyl)carbamoyl]benzyl}carbamoylmethoxy)-2,3,4,5-tetrahydro-1,4-benzothiazepine;
3,5-trans-1,1-dioxo-3-(S)-3-ethyl-3-butyl-4-hydroxy-5-(S)-5-phenyl-7-bromo-8-(N-{(R)-α-[N-(carboxymethyl)carbamoyl]benzyl}carbamoylmethoxy)-2,3,4,5-tetrahydro-1,4-benzothiazepine
3,5-trans-1,1-dioxo-3-(R)-3-ethyl-3-butyl-4-hydroxy-5-(R)-5-phenyl-7-bromo-8-(N-{(R)-α-[N-(carboxymethyl)carbamoyl]benzyl}carbamoylmethoxy)-2,3,4,5-tetrahydro-1,4-benzothiazepine;
3,5-trans-1,1-dioxo-3-ethyl-3-butyl-5-phenyl-7-methylthio-8-(N-{(R)-α-[N-(carboxymethyl)carbamoyl]benzyl}carbamoylmethoxy)-2,3,4,5-tetrahydro-1,4-benzothiazepine;
3,5-trans-1,1-dioxo-3-ethyl-3-butyl-5-phenyl-7-methylthio-8-(N-{(R)-α-[N-(2-sulphoethyl)carbamoyl]-4-hydroxybenzyl}carbamoylmethoxy)-2,3,4,5-tetrahydro-1,4-benzothiazepine ammonia salt;
1,1-dioxo-3-(S)-3-ethyl-3-butyl-5-(S)-5-phenyl-7-methylthio-8-(N-{(R)-α-[N-(carboxymethyl)carbamoyl]benzyl}carbamoylmethoxy)-2,3,4,5-tetrahydro-1,4-benzothiazepine diethylamine salt; and
1,1-dioxo-3-(R)-3-ethyl-3-butyl-5-(R)-5-phenyl-7-methylthio-8-(N-{(R)-α-[N-(carboxymethyl)carbamoyl]benzyl}carbamoylmethoxy)-2,3,4,5-tetrahydro-1,4-benzothiazepine diethylamine salt;
   or a pharmaceutically acceptable salt, solvate, solvate of such a salt or a prodrug thereof.

A particular IBAT inhibitor for use in combination with compounds of the present invention is selected from any one of Examples 1-4 of WO 03/022830, or a pharmaceutically acceptable salt, solvate, solvate of such a salt or a prodrug thereof, and the compounds of Examples 1-4 are incorporated herein by reference. Claims 1-8 of WO 03/022830 are also incorporated herein by reference. A particular IBAT inhibitor selected from WO 03/022830 for use in combination with compounds of the present invention is selected from any one of:
1,1-dioxo-3-butyl-3-ethyl-4-hydroxy-5-phenyl-7-(N-{(R)-α-[N-(carboxymethyl)carbamoyl]benzyl}carbamoylmethylthio)-2,3,4,5-tetrahydrobenzothiepine
1,1-dioxo-3-butyl-3-ethyl-4-hydroxy-5-phenyl-7-(N-{(R)-α-[N-(2-sulphoethyl)carbamoyl]-4-hydroxybenzyl}carbamoylmethylthio)-2,3,4,5-tetrahydrobenzothiepine ammonia salt
1,1-dioxo-3-butyl-3-ethyl-4-hydroxy-5-phenyl-7-{N-[α-(carboxy)-2-fluorobenzyl]carbamoylmethylthio}-2,3,4,5-tetrahydrobenzothiepine; and
1,1-dioxo-3-butyl-3-ethyl-4-hydroxy-5-phenyl-7-{N-[1-(carboxy)-1-(thien-2-yl)methyl]carbamoylmethylthio}-2,3,4,5-tetrahydrobenzothiepine
   or a pharmaceutically acceptable salt, solvate, solvate of such a salt or a prodrug thereof.

A particular IBAT inhibitor for use in combination with compounds of the present invention is selected from any one of Examples 1-39 of WO 03/022286, or a pharmaceutically acceptable salt, solvate, solvate of such a salt or a prodrug thereof, and the compounds of Examples 1-39 are incorporated herein by reference. Claims 1-10 of WO 03/022286 are also incorporated herein by reference. A particular IBAT inhibitor selected from WO 03/022286 for use in combination with compounds of the present invention is selected from any one of:
1,1-dioxo-3,3-dibutyl-5-phenyl-7-methylthio-8-(N-{(R)-α-[N-((R)-1-carboxy-2-methylthio-ethyl)carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-((S)-1-carboxy-2-(R)-hydroxypropyl)carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-((S)-1-carboxy-2-methylpropyl)carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-((S)-1-carboxybutyl)carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-((S)-1-carboxypropyl)carbamoyl]benzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-((S)-1-carboxyethyl)carbamoyl]benzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-((S)-1-carboxy-2-(R)-hydroxypropyl)carbamoyl]benzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-(2-sulphoethyl)carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-((S)-1-carboxyethyl)carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-((R)-1-carboxy-2-methylthioethyl)carbamoyl]benzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-{(S)-1-[N-((S)-2-hydroxy-1-carboxyethyl)carbamoyl]propyl}carbamoyl]benzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-((S)-1-carboxy-2-methylpropyl)carbamoyl]benzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-((S)-1-carboxypropyl)carbamoyl]-4-hydroxybenzyl}carbamoylmethoxy)-2,3,4,5-tetrahydro-1,2,5-benzothiadiazepine; and
1,1-dioxo-3,3-dibutyl-5-phenyl-7-methylthio-8-[N-((R)-α-carboxy-4-hydroxybenzyl)carbamoylmethoxy]-2,3,4,5-tetrahydro-1,2,5-benzothiadiazepine;
   or a pharmaceutically acceptable salt, solvate, solvate of such a salt or a prodrug thereof.

A particular IBAT inhibitor for use in combination with compounds of the present invention is selected from any one of Examples 1-7 of WO 03/091232, or a pharmaceutically acceptable salt, solvate, solvate of such a salt or a prodrug thereof, and the compounds of Examples 1-7 are incorporated herein by reference. Claims 1-10 of WO 03/091232 are also incorporated herein by reference. A particular IBAT inhibitor selected from WO 03/091232 for use in combination with compounds of the present invention is selected from any one of:
1,1-Dioxo-3,3-dibutyl-5-phenyl-7-methylthio-8-(N-{(R)-α[N-(2-(S)-3-(R)-4-(R)-5-(R)-2,3,4,5,6-pentahydroxyhexyl)carbamoyl}benzyl]carbamoylmethoxy)-2,3,4,5-tetrahydro-1,2,5-benzothiadiazepine;
1,1-Dioxo-3,3-dibutyl-5-phenyl-7-methylthio-8-(N-{(R)-α-[N-(2-(S)-3-(R)-4-(R)-5-(R)-2,3,4,5,6-pentahydroxyhexyl)carbamoyl]-4-hydroxybenzyl}carbamoylmethoxy)-2,3,4,5-tetrahydro-1,2,5-benzothiadiazepine;
1,1-Dioxo-3,3-dibutyl-5-phenyl-7-methylthio-8-[N-((R/S)-α-{N-[1-(R)-2-(S)-1-hydroxy-1-(3,4-dihydroxyphenyl)prop-2-yl]carbamoyl}-4-hydroxybenzyl)carbamoylmethoxy]-2,3,4,5-tetrahydro-1,2,5-benzothiadiazepine;
1,1-Dioxo-3,3-dibutyl-5-phenyl-7-methylthio-8-{N-[(R)-α-(N-{2-(S)-[N-(carbamoylmethyl)carbamoyl]pyrrolidin-1-ylcarbonylmethyl}carbamoyl)benzyl]carbamoylmethoxy}-2,3,4,5-tetrahydro-1,2,5-benzothiadiazepine;
1,1-Dioxo-3,3-dibutyl-5-phenyl-7-methylthio-8-[N-((R)-α-{N-[2-(3,4,5-trihydroxyphenyl)ethyl]carbamoyl}benzyl)carbamoylmethoxy]-2,3,4,5-tetrahydro-1,2,5-benzothiadiazepine; and
1,1-Dioxo-3,3-dibutyl-5-phenyl-7-methylthio-8-(N-{(R)-α-[N-(2-(R)-3-(S)-4-(S)-5-(R)-3,4,5,6-tetrahydroxytetrahydropyran-2-ylmethyl)carbamoyl]benzyl}carbamoylmethoxy)-2,3,4,5-tetrahydro-1,2,5-benzothiadiazepine;
   or a pharmaceutically acceptable salt, solvate, solvate of such a salt or a prodrug thereof. Further suitable compounds possessing IBAT inhibitory for use in combination with compounds of the present invention are disclosed in WO 03/106482

Suitable IBAT inhibitors having the above structure for use in combination with compounds of the present invention are selected from any one of:
1,1-dioxo-3,3-dibutyl-5-phenyl-7-methylthio-8-(N-{(R)-α-[N′-((S)-1-carboxyethyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-((S)-1-carboxypropyl)carbamoyl}benzyl]carbamoylmethoxy)-2,3,4,5-tetrahydro-1,5-benzothiazepine;
1,1-dioxo-3,3-dibutyl-5-phenyl-7-methylthio-8-(N-{(R)-α-[N′-((S)-1-carboxybutyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-((S)-1-carboxy-2-methylpropyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-((S)-1-carboxy-2-methylbutyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-((S)-1-carboxy-3-methylbutyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-((S)-1-carboxy-2-hydroxypropyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-((S)-1-carboxy-2-mesylethyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-((S)-1-carboxy-3-methylsulphonylpropyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-((S)-1-carboxy-3-mesylpropyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-((S)-1-carboxyethyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-((S)-1-carboxypropyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-((S)-1-carboxybutyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-((S)-1-carboxy-2-methylpropyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-((S)-1-carboxy-2-methylbutyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-((S)-1-carboxy-3-methylbutyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-((S)-1-carboxy-2-hydroxyethyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-((5)-1-carboxy-2-hydroxypropyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N-{(R)-α-α-[N′-((S)-1-carboxy-2-methylthioethyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-((S)-1-carboxy-2-methylsulphinylethyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-((S)-1-carboxy-2-mesylethyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-((S)-1-carboxy-2-methoxyethyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-((5)-1-carboxy-3-methylthiopropyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-((S)-1-carboxy-3-methylsulphonylpropyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-((S)-1-carboxy-3-mesylpropyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-((S)-1-carboxypropyl)carbamoyl]-4-hydroxybenzyl}carbamoylmethoxy)-2,3,4,5-tetrahydro-1,5-benzothiazepine; or
1,1-dioxo-3,3-dibutyl-5-phenyl-7-methylthio-8-(N-{(R)-α-[N′-((S)-1-carboxyethyl)carbamoyl]benzyl}carbamoylmethoxy)-2,3,4,5-tetrahydro-1,5-benzothiazepine.
   or a pharmaceutically acceptable salt, solvate, solvate of such a salt or a prodrug thereof.

Further suitable IBAT inhibitors for use in combination with compounds of the present invention are those disclosed in WO 04/076430.

In a particular aspect of the invention an IBAT inhibitor or a pharmaceutically acceptable salt, solvate, solvate of such a salt or a prodrug thereof is an IBAT inhibitor or a pharmaceutically acceptable salt thereof.

Therefore in an additional feature of the invention, there is provided a combination of a compound of formula (I), or a pharmaceutically acceptable salt, solvate, solvate of such a salt or a prodrug thereof and an IBAT inhibitor, or a pharmaceutically acceptable salt, solvate, solvate of such a salt or a prodrug thereof.

Therefore in an additional feature of the invention, there is provided a method for producing a cholesterol lowering effect in a warm-blooded animal, such as man, in need of such treatment which comprises administering to said animal an effective amount of a compound of formula (I), or a pharmaceutically acceptable salt, solvate, solvate of such a salt or a prodrug thereof in simultaneous, sequential or separate administration with an effective amount of an IBAT inhibitor, or a pharmaceutically acceptable salt, solvate, solvate of such a salt or a prodrug thereof.

According to a further aspect of the invention there is provided a pharmaceutical composition which comprises a compound of formula (I), or a pharmaceutically acceptable salt, solvate, solvate of such a salt or a prodrug thereof, and an IBAT inhibitor, or a pharmaceutically acceptable salt, solvate, solvate of such a salt or a prodrug thereof, in association with a pharmaceutically acceptable diluent or carrier.

According to a further aspect of the present invention there is provided a kit comprising a compound of formula (I), or a pharmaceutically acceptable salt, solvate, solvate of such a salt or a prodrug thereof, and an IBAT inhibitor, or a pharmaceutically acceptable salt, solvate, solvate of such a salt or a prodrug thereof.

According to a further aspect of the present invention there is provided a kit comprising:

a) a compound of formula (I), or a pharmaceutically acceptable salt, solvate, solvate of such a salt or a prodrug thereof, in a first unit dosage form;
b) an IBAT inhibitor, or a pharmaceutically acceptable salt, solvate, solvate of such a salt or a prodrug thereof; in a second unit dosage form; and
c) container means for containing said first and second dosage forms.

According to a further aspect of the present invention there is provided a kit comprising:

a) a compound of formula (I), or a pharmaceutically acceptable salt, solvate, solvate of such a salt or a prodrug thereof, together with a pharmaceutically acceptable diluent or carrier, in a first unit dosage form;
b) an IBAT inhibitor, or a pharmaceutically acceptable salt, solvate, solvate of such a salt or a prodrug thereof, in a second unit dosage form; and
c) container means for containing said first and second dosage forms.

According to another feature of the invention there is provided the use of a compound of the formula (I), or a pharmaceutically acceptable salt, solvate, solvate of such a salt or a prodrug thereof, and an IBAT inhibitor, or a pharmaceutically acceptable salt, solvate, solvate of such a salt or a prodrug thereof, in the manufacture of a medicament for use in the production of a cholesterol lowering effect in a warm-blooded animal, such as man.

According to a further aspect of the present invention there is provided a combination treatment comprising the administration of an effective amount of a compound of the formula (I), or a pharmaceutically acceptable salt, solvate, solvate of such a salt or a prodrug thereof, optionally together with a pharmaceutically acceptable diluent or carrier, with the simultaneous, sequential or separate administration of an effective amount of an IBAT inhibitor, or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

According to a further aspect of the present invention there is provided a combination treatment comprising the administration of an effective amount of a compound of the formula (I), or a pharmaceutically acceptable salt, solvate, solvate of such a salt or a prodrug thereof, optionally together with a pharmaceutically acceptable diluent or carrier, with the simultaneous, sequential or separate administration of an effective amount of an IBAT inhibitor, or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

In another aspect of the invention, the compound of formula (I), or a pharmaceutically acceptable salt, solvate, solvate of such a salt or a prodrug thereof, may be administered in association with a PPAR alpha and/or gamma and/or delta agonist, or pharmaceutically acceptable salts, solvates, solvates of such salts or prodrugs thereof. Suitable PPAR alpha and/or gamma and/or delta agonists, pharmaceutically acceptable salts, solvates, solvates of such salts or prodrugs thereof are well known in the art. These include the compounds described in WO 01/12187, WO 01/12612, WO 99/62870, WO 99/62872, WO 99/62871, WO 98/57941, WO 01/40170, WO 01/40172, WO 02/085844, WO 02/096863, WO03/051821, WO03/051822, WO03/051826, WO 04/000790, WO04/000295, WO04/000294, PCT/GB03/02584, PCT/GB03/02591, PCT/GB03/02598, J Med Chem, 1996, 39, 665, Expert Opinion on Therapeutic Patents, 10 (5), 623-634 (in particular the compounds described in the patent applications listed on page 634) and J Med Chem, 2000, 43, 527 which are all incorporated herein by reference. Particularly a PPAR alpha and/or gamma and/or delta agonist refers to muraglitazar (BMS 298585), rivoglitazone (CS-011), netoglitazone (MCC-555), balaglitazone (DRF-2593, N,N-2344), clofibrate, fenofibrate, bezafibrate, gemfibrozil, ciprofibrate, beclofibrate, etofibrate, gemcabene, pioglitazone, rosiglitazone, edaglitazone, LY-293111, MBX-2044, AVE-0847, AVE-8134, CLX-0921, DRF-10945, DRF-4832, LY-518674, naveglitazar (LY-818), LY-929, 641597, GW-590735, GW-677954, GW-501516, metaglidazen (MBX-102), T-131, SDX-101 E-3030, PLX-204, ONO-5129, KRP-101, R-483 (BM131258), TAK-559, K-111 (BM170744), netoglitazone (MCC-555; RWJ-241947; isaglitazone), FK-614 or TAK-654

Particularly a PPAR alpha and/or gamma and/or delta agonist refers to (S)-2-ethoxy-3-[4-(2-{4-methanesulphonyloxyphenyl}ethoxy)phenyl]propanoic acid (tesaglitazar) and pharmaceutically acceptable salts thereof.

Therefore in an additional feature of the invention, there is provided a combination of a compound of formula (I), or a pharmaceutically acceptable salt, solvate, solvate of such a salt or a prodrug thereof and a PPAR alpha and/or gamma agonist, or a pharmaceutically acceptable salt, solvate, solvate of such a salt or a prodrug thereof.

Therefore in an additional feature of the invention, there is provided a method for producing a cholesterol lowering effect in a warm-blooded animal, such as man, in need of such treatment which comprises administering to said animal an effective amount of a compound of formula (I), or a pharmaceutically acceptable salt, solvate, solvate of such a salt or a prodrug thereof in simultaneous, sequential or separate administration with an effective amount of a PPAR alpha and/or gamma and/or delta agonist, or a pharmaceutically acceptable salt, solvate, solvate of such a salt or a prodrug thereof.

According to a further aspect of the invention there is provided a pharmaceutical composition which comprises a compound of formula (I), or a pharmaceutically acceptable salt, solvate, solvate of such a salt or a prodrug thereof, and a PPAR alpha and/or gamma and/or delta agonist, or a pharmaceutically acceptable salt, solvate, solvate of such a salt or a prodrug thereof, in association with a pharmaceutically acceptable diluent or carrier.

According to a further aspect of the present invention there is provided a kit comprising a compound of formula (I), or a pharmaceutically acceptable salt, solvate, solvate of such a salt or a prodrug thereof, and a PPAR alpha and/or gamma and/or delta agonist, or a pharmaceutically acceptable salt, solvate, solvate of such a salt or a prodrug thereof.

According to a further aspect of the present invention there is provided a kit comprising:

a) a compound of formula (I), or a pharmaceutically acceptable salt, solvate, solvate of such a salt or a prodrug thereof, in a first unit dosage form;
b) a PPAR alpha and/or gamma and/or delta agonist, or a pharmaceutically acceptable salt, solvate, solvate of such a salt or a prodrug thereof; in a second unit dosage form; and
c) container means for containing said first and second dosage forms.

According to a further aspect of the present invention there is provided a kit comprising:

a) a compound of formula (I), or a pharmaceutically acceptable salt, solvate, solvate of such a salt or a prodrug thereof, together with a pharmaceutically acceptable diluent or carrier, in a first unit dosage form;
b) a PPAR alpha and/or gamma and/or delta agonist, or a pharmaceutically acceptable salt, solvate, solvate of such a salt or a prodrug thereof, in a second unit dosage form; and
c) container means for containing said first and second dosage forms.

According to another feature of the invention there is provided the use of a compound of the formula (I), or a pharmaceutically acceptable salt, solvate, solvate of such a salt or a prodrug thereof, and a PPAR alpha and/or gamma and/or delta agonist, or a pharmaceutically acceptable salt, solvate, solvate of such a salt or a prodrug thereof, in the manufacture of a medicament for use in producing a cholesterol lowering effect in a warm-blooded animal, such as man.

According to a further aspect of the present invention there is provided a combination treatment comprising the administration of an effective amount of a compound of the formula (I), or a pharmaceutically acceptable salt, solvate, solvate of such a salt or a prodrug thereof, optionally together with a pharmaceutically acceptable diluent or carrier, with the simultaneous, sequential or separate administration of an effective amount of a PPAR alpha and/or gamma and/or delta agonist, or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

In another aspect of the invention, there is provided a combination treatment comprising the administration of an effective amount of a compound of the formula (I), or a pharmaceutically acceptable salt, solvate, solvate of such a salt or a prodrug thereof, optionally together with a pharmaceutically acceptable diluent or carrier, with the simultaneous, sequential or separate administration of an -agonists to the receptor HM74A (nicotinic acid receptor). HM74A receptor agonists may be nicotine acid derivates. As used herein “nicotinic acid derivative” means a compounds comprising a pyridine-3-carboxylate structure or a pyrazine-2-carboxylate structure. Examples of nicotinic acid derivatives include nicotinic acid, niceritrol, nicofuranose, NIASPAN® and acipimox.

HM74A receptor agonists may be anthranilic acid derivatives described in WO-2005016867 and WO-2005016870.

Other nicotinic receptor agonists are for example compounds described in WO2005011677, WO2004032928 and WO2004033431.

Therefore, in an additional feature of the invention, there is provided a combination of a compound of formula (I), or a pharmaceutically acceptable salt, solvate, solvate of such a salt or a prodrug thereof and a HM74A receptor agonists or a pharmaceutically acceptable salt, solvate, solvate of such a salt or a prodrug thereof.

Therefore in an additional feature of the invention, there is provided a method for producing a cholesterol lowering effect in a warm-blooded animal, such as man, in need of such treatment which comprises administering to said animal an effective amount of a compound of formula (I), or a pharmaceutically acceptable salt, solvate, solvate of such a salt or a prodrug thereof in simultaneous, sequential or separate administration with an effective amount of a HM74A receptor agonists, or a pharmaceutically acceptable salt, solvate, solvate of such a salt or a prodrug thereof.

According to a further aspect of the invention there is provided a pharmaceutical composition which comprises a compound of formula (I), or a pharmaceutically acceptable salt, solvate, solvate of such a salt or a prodrug thereof, and a HM74A receptor agonists, or a pharmaceutically acceptable salt, solvate, solvate of such a salt or a prodrug thereof, in association with a pharmaceutically acceptable diluent or carrier.

In another aspect of the invention, there is provided a combination treatment comprising the administration of an effective amount of a compound of the formula (I), or a pharmaceutically acceptable salt, solvate, solvate of such a salt or a prodrug thereof, optionally together with a pharmaceutically acceptable diluent or carrier, with the simultaneous, sequential or separate administration of a mediator of reverse cholesterol transport i.e. a peptide (Apo A-1 mimetic peptides) or small molecule mediator of reverse cholesterol transport e.g. those described in Circ. 2002; 105:290, Circ. 2004.109:3215, Curr. Opinion in Lipidology 2004, 15:645 or in WO2004094471.

In another aspect of the invention, the compound of formula I, or a pharmaceutically acceptable salt or solvate thereof, or a solvate of such a salt, may be administered in association with an anti-obesity compound, or pharmaceutically acceptable salts, solvates, solvates of such salts or prodrugs thereof, for example a pancreatic lipase inhibitor e.g. orlistat (EP 129,748) or an appetite (satiety) controlling substance for example sibutramine (GB 2,184,122 and U.S. Pat. No. 4,929,629), a cannabinoid 1 (CB1) antagonist or inverse agonist, or pharmaceutically acceptable salts, solvates, solvates of such salts or prodrugs thereof, for example rimonabant (EP 656354) and as described in WO01/70700 or a melanin concentrating hormone (MCH) antagonist, or pharmaceutically acceptable salts, solvates, solvates of such salts or prodrugs thereof, for example as described in WO 04/004726.

According to another feature of the invention there is provided the use of a compound of the formula (I), or a pharmaceutically acceptable salt, solvate, solvate of such a salt or a prodrug thereof, and a nicotinic acid derivative, or a pharmaceutically acceptable salt, solvate, solvate of such a salt or a prodrug thereof, in the manufacture of a medicament for use in the production of a cholesterol lowering effect in a warm-blooded animal, such as man.

In another aspect of the invention, the compound of formula (I), or a pharmaceutically acceptable salt, solvate, solvate of such a salt or a prodrug thereof, may be administered in association with a bile acid sequestrant or a pharmaceutically acceptable salt, solvate, solvate of such a salt or a prodrug thereof. Suitable bile acid sequestrants include cholestyramine, cholestipol and cosevelam hydrochloride.

Therefore, in an additional feature of the invention, there is provided a combination of a compound of formula (I), or a pharmaceutically acceptable salt, solvate, solvate of such a salt or a prodrug thereof and a bile acid sequestrant or a pharmaceutically acceptable salt, solvate, solvate of such a salt or a prodrug thereof.

Therefore in an additional feature of the invention, there is provided a method for producing a cholesterol lowering effect in a warm-blooded animal, such as man, in need of such treatment which comprises administering to said animal an effective amount of a compound of formula (I), or a pharmaceutically acceptable salt, solvate, solvate of such a salt or a prodrug thereof in simultaneous, sequential or separate administration with an effective amount of a bile acid sequestrant, or a pharmaceutically acceptable salt, solvate, solvate of such a salt or a prodrug thereof.

According to a further aspect of the invention there is provided a pharmaceutical composition which comprises a compound of formula (I), or a pharmaceutically acceptable salt, solvate, solvate of such a salt or a prodrug thereof, and a bile acid sequestrant, or a pharmaceutically acceptable salt, solvate, solvate of such a salt or a prodrug thereof, in association with a pharmaceutically acceptable diluent or carrier.

According to another feature of the invention there is provided the use of a compound of the formula (I), or a pharmaceutically acceptable salt, solvate, solvate of such a salt or a prodrug thereof, and a bile acid sequestrant, or a pharmaceutically acceptable salt, solvate, solvate of such a salt or a prodrug thereof, in the manufacture of a medicament for use in the production of a cholesterol lowering effect in a warm-blooded animal, such as man.

In another aspect of the invention, the compound of formula I, or a pharmaceutically acceptable salt or solvate thereof, or a solvate of such a salt, may be administered in association with a cholesteryl ester transfer protein (CETP) inhibitor, or pharmaceutically acceptable salts, solvates, solvates of such salts or prodrugs thereof, for example TIT-705, torcetrapib (CP-529414), Bay 194789 and those referenced and described in WO05033082 or WO 00/38725 page 7 line 22-page 10, line 17 which are incorporated herein by reference.

In another aspect of the invention, the compound of formula I, or a pharmaceutically acceptable salt or solvate thereof, or a solvate of such a salt, may be administered in association with a acyl coenzymA: cholesterol O-acyltransferase (ACAT) inhibitor, or pharmaceutically acceptable salts, solvates, solvates of such salts or prodrugs thereof, for example pactimibe (CS-505), eflucimibe (F-12511) and SMP-797, avasimibe or K604.

In yet another aspect of the invention, the compound of formula I, association with modulators for example GW-4064 and INT-747 of nuclear receptors such as farnesoid or a pharmaceutically acceptable salt or solvate thereof, or a solvate of such a salt, may be administered in X receptor (FXR), or pharmaceutically acceptable salt, solvate, solvate of such a salt or a prodrug thereof.

In another aspect of the invention, the compound of formula I, or a pharmaceutically acceptable salt or solvate thereof, or a solvate of such a salt, may be administered in association with a phytosterol compound, or pharmaceutically acceptable salts, solvates, solvates of such salts or prodrugs thereof, for example stanols. An example of phytosterol analogs is FM-VP4.

In another aspect of the invention, the compound of formula I, or a pharmaceutically acceptable salt or solvate thereof, or a solvate of such a salt, may be administered in association with other therapies for the treatment of metabolic syndrome or type 2 diabetes and its associated complications, these include biguanide drugs, for example metformin, phenformin and buformin, insulin (synthetic insulin analogues, amylin) and oral antihyperglycemics (these are divided into prandial glucose regulators and alpha-glucosidase inhibitors). An example of an alpha-glucosidase inhibitor is acarbose or voglibose or miglitol. An example of a prandial glucose regulator is repaglinide or nateglinide.

In another aspect of the invention, the compound of formula I, or a pharmaceutically acceptable salt or solvate thereof, or a solvate of such a salt, may be administered in association with a sulfonylurea for example: glimepiride, glibenclamide (glyburide), gliclazide, glipizide, gliquidone, chloropropamide, tolbutamide, acetohexamide, glycopyramide, carbutamide, glibonuride, glisoxepid, glybuthiazole, glibuzole, glyhexamide, glymidine, glypinanaide, phenbutamide, tolcylamide and tolazamide. Preferably the sulfonylurea is glimepiride or glibenclamide (glyburide). More preferably the sulfonylurea is glimepiride. Therefore the present invention includes administration of a compound of the present invention in conjunction with one, two or more existing therapies described in this paragraph. The doses of the other existing therapies for the treatment of type 2 diabetes and its associated complications will be those known in the art and approved for use by regulatory bodies for example the FDA and may be found in the Orange Book published by the FDA. Alternatively smaller doses may be used as a result of the benefits derived from the combination.

According to an additional further aspect of the present invention there is provided a combination treatment comprising the administration of an effective amount of a compound of the formula (I), or a pharmaceutically acceptable salt, solvate, solvate of such a salt or a prodrug thereof, optionally together with a pharmaceutically acceptable diluent or carrier, with the simultaneous, sequential or separate administration one or more of the following agents selected from Group X:
an antihypertensive compound (for example althiazide, benzthiazide, captopril, carvedilol, chlorothiazide sodium, clonidine hydrochloride, cyclothiazide, delapril hydrochloride, dilevalol hydrochloride, doxazosin mesylate, fosinopril sodium, guanfacine hydrochloride, methyldopa, metoprolol succinate, moexipril hydrochloride, monatepil maleate, pelanserin hydrochloride, phenoxybenzemine hydrochloride, prazosin hydrochloride, primidolol, quinapril hydrochloride, quinaprilat, ramipril, terazosin hydrochloride, candesartan, candesartan cilexetil, telmisartan, amlodipine besylate, amlodipine maleate and bevantolol hydrochloride);
an angiotensin converting enzyme inhibitor (for example alacepril, alatriopril, altiopril calcium, ancovenin, benazepril, benazepril hydrochloride, benazeprilat, benzoylcaptopril, captopril, captopril-cysteine, captopril-glutathione, ceranopril, ceranopril, ceronapril, cilazapril, cilazaprilat, delapril, delapril-diacid, enalapril, enalaprilat, enapril, epicaptopril, foroxymithine, fosfenopril, fosenopril, fosenopril sodium, fosinopril, fosinopril sodium, fosinoprilat, fosinoprilic acid, glycopril, hemorphin-4, idrapril, imidapril, indolapril, indolaprilat, libenzapril, lisinopril, lyciumin A, lyciumin B, mixanpril, moexipril, moexiprilat, moveltipril, muracein A, muracein B, muracein C, pentopril, perindopril, perindoprilat, pivalopril, pivopril, quinapril, quinapril hydrochloride, quinaprilat, ramipril, ramiprilat, spirapril, spirapril hydrochloride, spiraprilat, spiropril, spiropril hydrochloride, temocapril, temocapril hydrochloride, teprotide, trandolapril, traridolaprilat, utibapril, zabicipril, zabiciprilat, zofenopril and zofenoprilat);
an angiotensin II receptor antagonist (for example candesartan, candesartan cilexetil, losartan, valsartan, irbesartan, tasosartan, telmisartan and eprosartan);
an andrenergic blocker (for example bretylium tosylate, dihydroergotamine so mesylate, phentolamine mesylate, solypertine tartrate, zolertine hydrochloride, carvedilol or labetalol hydrochloride); an alpha andrenergic blocker (for example fenspiride hydrochloride, labetalol hydrochloride, proroxan and alfuzosin hydrochloride); a beta andrenergic blocker (for example acebutolol, acebutolol hydrochloride, alprenolol hydrochloride, atenolol, bunolol hydrochloride, carteolol hydrochloride, celiprolol hydrochloride, cetamolol hydrochloride, cicloprolol hydrochloride, dexpropranolol hydrochloride, diacetolol hydrochloride, dilevalol hydrochloride, esmolol hydrochloride, exaprolol hydrochloride, flestolol sulfate, labetalol hydrochloride, levobetaxolol hydrochloride, levobunolol hydrochloride, metalol hydrochloride, metoprolol, metoprolol tartrate, nadolol, pamatolol sulfate, penbutolol sulfate, practolol, propranolol hydrochloride, sotalol hydrochloride, timolol, timolol maleate, tiprenolol hydrochloride, tolamolol, bisoprolol, bisoprolol fumarate and nebivolol); or a mixed alpha/beta andrenergic blocker;
an andrenergic stimulant (for example combination product of chlorothiazide and methyldopa, the combination product of methyldopa hydrochlorothiazide and methyldopa, clonidine hydrochloride, clonidine, the combination product of chlorthalidone and clonidine hydrochloride and guanfacine hydrochloride);
channel blocker, for example a calcium channel blocker (for example clentiazem maleate, amlodipine besylate, isradipine, nimodipine, felodipine, nilvadipine, nifedipine, teludipine hydrochloride, diltiazem hydrochloride, belfosdil, verapamil hydrochloride or fostedil);
a diuretic (for example the combination product of hydrochlorothiazide and spironolactone and the combination product of hydrochlorothiazide and triamterene);
anti-anginal agents (for example amlodipine besylate, amlodipine maleate, betaxolol hydrochloride, bevantolol hydrochloride, butoprozine hydrochloride, carvedilol, cinepazet maleate, metoprolol succinate, molsidomine, monatepil maleate, primidolol, ranolazine hydrochloride, tosifen or verapamil hydrochloride);
vasodilators for example coronary vasodilators (for example fostedil, azaclorzine hydrochloride, chromonar hydrochloride, clonitrate, diltiazem hydrochloride, dipyridamole, droprenilamine, erythrityl tetranitrate, isosorbide dinitrate, isosorbide mononitrate, lidoflazine, mioflazine hydrochloride, mixidine, molsidomine, nicorandil, nifedipine, nisoldipine, nitroglycerine, oxprenolol hydrochloride, pentrinitrol, perhexyline maleate, prenylamine, propatyl nitrate, terodiline hydrochloride, tolamolol and verapamil);
anti-coagulants (selected from argatroban, bivalirudin, dalteparin sodium, desirudin, dicumarol, Iyapolate sodium, nafamostat mesylate, phenprocoumon, tinzaparin sodium and warfarin sodium);
antithrombotic agents (for example anagrelide hydrochloride, bivalirudin, cilostazol, dalteparin sodium, danaparoid sodium, dazoxiben hydrochloride, efegatran sulfate, enoxaparin sodium, fluretofen, ifetroban, ifetroban sodium, lamifiban, lotrafiban hydrochloride, napsagatran, orbofiban acetate, roxifiban acetate, sibrafiban, tinzaparin sodium, trifenagrel, abciximab and zolimomab aritox);
fibrinogen receptor antagonists (for example roxifiban acetate, fradafiban, orbofiban, lotrafiban hydrochloride, tirofiban, xemilofiban, monoclonal antibody 7E3 and sibrafiban)
platelet inhibitors (for example cilostezol, clopidogrel bisulfate, epoprostenol, epoprostenol sodium, ticlopidine hydrochloride, aspirin, ibuprofen, naproxen, sulindae, indomethacin, mefenamate, droxicam, diclofenac, sulfinpyrazone and piroxicam, dipyridamole);
platelet aggregation inhibitors (for example acadesine, beraprost, beraprost sodium, ciprostene calcium, itezigrel, lifarizine, lotrafiban hydrochloride, orbofiban acetate, oxagrelate, fradafiban, orbofiban, tirofiban and xemilofiban)
hemorrheologic agents (for example pentoxifylline);
lipoprotein associated coagulation inhibitors;
Factor VIIa inhibitors;
Factor Xa inhibitors;
low molecular weight heparins (for example enoxaparin, nardroparin, dalteparin, certroparin, parnaparin, reviparin and tinzaparin);
liver X receptor (OCR) agonists for example GW-3965 and those described in WO00224632, WO00103705, WO02090375 and WO00054759 (claim 1 and the named examples of these four application are incorporated herein by reference);
microsomal triglyceride transfer protein inhibitors for example implitapide, CP-346086, JTT-130, BMS-201038, R-103757 and those described in WO05/021486, WO03004020, WO03002533, WO02083658 and WO 00242291 (claim 1 and the named examples of these four application are incorporated herein by reference);
ApoA1 expression inducer for example those described in WO2005032559 or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

Therefore, in an additional feature of the invention, there is provided a combination of a compound of formula (I), or a pharmaceutically acceptable salt, solvate, solvate of such a salt or a prodrug thereof and a compound from Group X or a pharmaceutically acceptable salt, solvate, solvate of such a salt or a prodrug thereof.

Therefore in an additional feature of the invention, there is provided a method for producing a cholesterol lowering effect in a warm-blooded animal, such as man, in need of such treatment which comprises administering to said animal an effective amount of a compound of formula (I), or a pharmaceutically acceptable salt, solvate, solvate of such a salt or a prodrug thereof in simultaneous, sequential or separate administration with an effective amount of a compound from Group X, or a pharmaceutically acceptable salt, solvate, solvate of such a salt or a prodrug thereof.

According to a further aspect of the invention there is provided a pharmaceutical composition which comprises a compound of formula (I), or a pharmaceutically acceptable salt, solvate, solvate of such a salt or a prodrug thereof, and a compound from Group X, or a pharmaceutically acceptable salt, solvate, solvate of such a salt or a prodrug thereof, in association with a pharmaceutically acceptable diluent or carrier.

According to another feature of the invention there is provided the use of a compound of the formula (I), or a pharmaceutically acceptable salt, solvate, solvate of such a salt or a prodrug thereof, and a compound from Group X, or a pharmaceutically acceptable salt, solvate, solvate of such a salt or a prodrug thereof, in the manufacture of a medicament for use in the production of a cholesterol lowering effect in a warm-blooded animal, such as man.

In addition to their use in therapeutic medicine, the compounds of formula (I), or a pharmaceutically acceptable salt, solvate, solvate of such a salt or a prodrug thereof, are also useful as pharmacological tools in the development and standardisation of in vitro and in vivo test systems for the evaluation of the effects of inhibitors of cholesterol absorption in laboratory animals such as cats, dogs, rabbits, monkeys, rats and mice, as part of the search for new therapeutic agents.

In the above other pharmaceutical composition, process, method, use and medicament manufacture features, the alternative and preferred embodiments of the compounds of the invention described herein also apply.

### EXAMPLES

The invention will now be illustrated in the following non limiting Examples, in which standard techniques known to the skilled chemist and techniques analogous to those described in these Examples may be used where appropriate, and in which, unless otherwise stated:

(i) evaporations were carried out by rotary evaporation in vacuo and work up procedures were carried out after removal of residual solids such as drying agents by filtration;
(ii) all reactions were carried out under an inert atmosphere at ambient temperature, typically in the range 18-25° C., with solvents of HPLC grade under anhydrous conditions, unless otherwise stated;
(iii) column chromatography (by the flash procedure) was performed on Silica gel 40-63 μm (Merck);
(iv) yields are given for illustration only and are not necessarily the maximum attainable;
(v) the structures of the end products of the formula (I) were generally confirmed by nuclear (generally proton) magnetic resonance (NMR) and mass spectral techniques; magnetic resonance chemical shift values were measured in deuterated CDCl₃ (unless otherwise stated) on the delta scale (ppm downfield from tetramethylsilane); proton data is quoted unless otherwise stated; spectra were recorded on a Varian Mercury-300 MHz, Varian Unity plus-400 MHz, Varian Unity plus-600 MHz or on Varian Inova-500 MHz spectrometer unless otherwise stated data was recorded at 400 MHz; and peak multiplicities are shown as follows: s, singlet; d, doublet; dd, double doublet; t, triplet; tt, triple triplet; q, quartet; tq, triple quartet; m, multiplet; br, broad; ABq, AB quartet; ABd, AB doublet, ABdd, AB doublet of doublets; dABq, doublet of AB quartets;

Mass spectra were recorded on one of the following instruments: LCT, QTOF, ZQ Mass spectrometer, all from Waters.

### LC-MS:

Separation was performed using Agilent 1100 Series Modules or Waters 1525 pump on a Synergi MAX-RP (Phenomenex) C12 3×50 mm 4 μm with gradient elution. Samples were injected using Waters 2700 Sample Manager.

### Mobile Phases:

Generic gradients were applied from 5% to 95% acetonitrile. Buffers containing 10 mM ammonium acetate or 5 mM ammonium formiate/5 mM formic acid were used.

The mass spectra were recorded with a Waters ZQ2000 or Waters ZMD equipped with an electrospray interface, switching positive and negative ionization mode. UV spectra were collected by a Aglent 1100 PDA or Waters 2996 DAD and the evaporative light scattering (ELS) signal by a Sedere Sedex 55 or 75.

Data collection and evaluation were performed using the MassLynx software. Accurate mass data were determined using either a LCT or QTOF MS (Waters) with leucine enkephaline (m/z 556.2771) as lockmass. Unless otherwise stated the mass ion quoted is (MH⁺).

Unless further details are specified in the text, analytical high performance liquid chromatography (HPLC) was performed on Prep LC 2000 (Waters), Cromasil Cg, 7 μm, (Akzo Nobel); MeCN and de-ionised water 10 mM ammonium acetate as mobile phases, with suitable composition;

(vii) intermediates were not generally fully characterised and purity was assessed by thin layer chromatography (TLC), HPLC, infra-red (IR), MS or NMR analysis;
(viii) where solutions were dried sodium sulphate was the drying agent; and
(ix) the following abbreviations may be used hereinbefore or hereinafter:—
DCM dichloromethane;
DMF N,N-dimethylformamide;
TBTU o-Benzotriazol-1-yl-N,N,N′,N′-tetramethyluronium tetrafluoroborate;
EtOAc ethyl acetate;
MeCN acetonitrile;
TFA trifluoroacetic acid;
DMAP 4-(dimethylamino)pyridine;
BSA N,O-Bis(trimethylsilyl)acetamide; and
TBAF tetrabutylammonium fluoride;
NMM N-methyl morpholine;
TEA triethylamine;
DBN 1,5-diazabicyclo-[4,3,0]-non-5-ene.

### Examples

### Example 1

### N²-{[4-((2R,3R)-1-(4-fluorophenyl)-3-{[2-(4-fluorophenyl)-2-hydroxyethyl]thio}-4-oxoazetidin-2-yl)phenoxy]acetyl}-N-(2-hydroxyethyl)-D-valinamide

N-{[4-((2R,3R)-1-(4-fluorophenyl)-3-{[2-(4-fluorophenyl)-2-oxoethyl]thio}-4-oxoazetidin-2-yl)phenoxy]acetyl}-D-valine (19 mg, 0.033 mmol), N-methylmorpholine (10 μl, 0.09 mmol) and TBTU (14 mg, 0.042 mmol) were added to a mixture of CH₂Cl₂ (2 ml) and DMF (0.2 ml). The mixture was stirred for 10 min at r.t. and aminoethanol (8 μl, 0.13 mmol) was added. The mixture was stirred for 30 rain. An additional amount aminoethanol (8 μl, 0.13 mmol) and TBTU (109 mg, 0.34 mmol) were added during 6 h. The mixture was purified by column chromatography on silica gel using methylene chloride/ethyl acetate (10/3) as eluent. The solvent was evaporated under reduced pressure. The yield was solved in methanol (1 ml) and NaBH₄ (5 mg, 0.13 mmol) was added. The reaction mixture was stirred at r.t for 30 min. A small amount of ammonium acetate buffer was added and the methanol was evaporated off. The residue was purified by preparative HPLC using acetonitril/ammonium acetate buffer (45:55) as eluent. The collected fractions were lyophilized to obtain the title compound.

(¹H-NMR, 400 MHz, CD₃OD): 0.85-0.95 (m, 6H), 2.05-2.1 (m, 1H), 2.9-3.05 (m, 2H), 3.55-3.65 (m, 2H), 4.05-4.3 (m, 2H), 4.6 (s, 2H), 4.9-4.95 (m, 1H), 6.95-7.1 (m, 6H), 7.25-7.4 (m, 6H)

### Example 2

### 1-deoxy-1-[(N-{[4-((2R,3R)-1-(4-fluorophenyl)-3-{[2-(4-fluorophenyl)-2-hydroxyethyl]thio}-4-oxoazetidin-2-yl)phenoxy]acetyl}glycyl)amino]-D-glucitol

N-{[4-((2R,3R)-1-(4-fluorophenyl)-3-{[2-(4-fluorophenyl)-2-oxoethyl]thio}-4-oxoazetidin-2-yl)phenoxy]acetyl}glycine (15 mg, 0.028), D-glucamin (8 mg, 0.042 mmol), N-methylmorpholine (5 μl, 0.049 mmol) and TBTU (12 mg, 0.039 mmol) were added to methylene chloride (2 ml) and DMF (0.2 ml) and the reaction mixture was stirred for 15 h. at r.t. The solvents were evaporated under reduced pressure and the residue was solved in methanol (1 ml) and NaBH₄ (40 mg, 1.06 mmol) was added. The reaction mixture was stirred at r.t for 1.5 h at r.t. A small amount of ammonium acetate buffer was added and the methanol was evaporated off. The residue was purified by preparative HPLC using acetonitril/ammonium acetate buffer (40:60) as eluent. The collected fractions were lyophilized to obtain the title compound.

(¹H-NMR, 400 MHz, CD₃OD): 2.9-3.1 (m, 2H), 3.45-3.85 (m, 6H), 4.9 (s, 2H), 4.0-4.05 (m, 1H), 4.6 (s, 2H), 4.9-4.95 (m, 1H), 6.95-7.1 (m, 6H), 7.25-7.4 (m, 6H)

### Example 3

### (3R)-3-[(N-{[4-((2R,3R)-1-(4-fluorophenyl)-3-{[2-(4-fluorophenyl)-2-hydroxyethyl]thio}-4-oxoazetidin-2-yl)phenoxy]acetyl}glycyl)amino]-4-phenylbutanoic acid

N-{[4-((2R,3R)-1-(4-fluorophenyl)-3-([2-(4-fluorophenyl)-2-oxoethyl]thiol-4-oxcazetidin-2-yl)phenoxy]acetyl}glycine (20 mg, 0.037), N-methylmorpholine (10 μl, 0.089 mmol) and tert-butyl (3R)-3-amino-4-phenylbutanoate (17 mg, 0.074 mmol) were added to methylene chloride (2 ml) and TBTU (13 mg, 0.041 mmol) was added. The reaction mixture was stirred for 5 h. at r.t. and the mixture was purified by column chromatography on silica gel using methylene chloride/ethyl acetate (1/1) as eluent. The solvents were evaporated off and to the residue was added formic acid (2 ml) and the mixture was stirred for 2 h. at r.t. The formic acid was evaporated under reduced pressure and was co-evaporated twice with toluene. The residue was solved in methanol (2 ml) and NaBH₄ (13 mg, 0.34 mmol) was added. The reaction mixture was stirred at r.t for 1 h. A small amount of ammonium acetate buffer was added and the methanol was evaporated off. The residue was purified by preparative HPLC using acetonitril/ammonium acetate buffer (44:55) as eluent. The collected fractions were lyophilized to obtain the title compound.

(¹H-NMR, 400 MHz, CD₃OD): 2.4 (t, 2H), 2.8 (t, 2H), 2.9-3.05 (m, 2H), 3.85 (d, 2H), 4.05 (d, 1H), 4.4 (t, 1H), 4.55 (s, 1H), 4.9 (d, 1H), 6.95-7.1 (m, 6H), 7.15-7.4 (m, 11H).

### Example 4

### 3-cyclohexyl-3-[(N-{[4-((2R,3R)-1-(4-fluorophenyl)-3-{[2-hydroxy-2-(4-methoxyphenyl)ethyl]thio}-4-oxoazetidin-2-yl)phenoxy]acetyl}glycyl)amino]propanoic acid

3-cyclohexyl-3-[(N-{[4-((2R,3R)-1-(4-fluorophenyl)-3-{[2-(4-methoxyphenyl)-2-oxoethyl]thio}-4-oxoazetidin-2-yl)phenoxy]acetyl}glycyl)amino]propanoic acid was dissolved in methanol (2 mL). NaBH₄ (0.0028 g, 0.074 mmole) was added and when the reaction was complete according to LC-MS a few drops of acetic acid was added. The solvent was removed under reduced pressure and the residue was purified by preparative HPLC on a Kromasil C8—column using a stepwise gradient of 38%, 43% then 52.5% MeCN in 0.1M ammonium acetate buffer as eluent. After freeze-drying, the desired product was obtained. NMR (500 MHz, CD₃COOD) 0.93-1.09 (m, 2H), 1.09-1.34 (m, 3H), 1.46-1.56 (m, 1H), 1.66 (brd, 1H), 1.71-1.81 (m, 4H), 2.38-2.46 (m, 1H), 2.47-2.54 (m, 1H), 2.92-3.09 (m, 2H), 3.77 (s, 1.5H), 3.78 (s, 1.5H), 3.89-3.98 (m, 3H), 4.04-4.11 (m, 1H), 4.61 (s, 2H), 4.74-4.79 (m, 1H), 4.83-4.89 (m, 1H), 6.81-6.85 (m, 2H), 6.99-7.09 (m, 4H), 7.22:7.31 (m, 4H), 7.31-7.37 (m, 2H)

### Example 5

### 2-[(N-{[4-((2R,3R)-1-(4-fluorophenyl)-3-{[2-(4-fluorophenyl)-2-hydroxyethyl]thio}-4-oxoazetidin-2-yl)phenoxy]acetyl}glycyl)amino]ethanesulfonic acid

[4-((2R,3R)-1-(4-fluorophenyl)-3-{[2-(4-fluorophenyl)-2-oxoethyl]thio}-4-oxoazetidin-2-yl)phenoxy]acetic acid (20 mg, 0.0414 mmol), 2-(glycylamino)ethanesulfonic acid (11 mg, 0.060 mmol) and N-methylmorpholine (30 mg, 0.30 mmol) were dissolved in DMF (0.5 ml). TBTU was added and the mixture was stirred for 2 h at room temperature. The solvent was evaporated under reduced pressure. The residue was dissolved in methanol (0.5 ml). NaBH4 (8 mg, 0.21 mmol) was added and the mixture was stirred for 10 min. The reaction mixture was evaporated under reduced pressure. The residue was purified by preparative HPLC using acetonitrile/ammonium acetate buffer (35:65) as eluent. After freeze-drying the title compound (as ammonium salt) was obtained.

¹H-NMR, 300 MHz, DMSO): 2.88-2.92 (m, 2H), 3.66 (d, 2H), 4.29 (m, 1H), 4.52 (s, 1H), 5.75 (m, 1H), 5.05 (m, 1H), 5.64 (t, 1H), 6.90-7.40 (m, 12H), 7.95 (t, 1H), 8.38 (t, 1H).

### Example 6 and 7

### N-{[4-((2R,3R)-1-(4-chlorophenyl)-3-{[(2R)-2-(4-chlorophenyl)-2-hydroxyethyl]thio}-4-oxoazetidin-2-yl)phenoxy]acetyl}glycyl-β-alanine and

### N-{[4-((2R,3R)-1-(4-chlorophenyl)-3-{[(2R)-2-(4-chlorophenyl)-2-hydroxyethyl]thio}-4-oxoazetidin-2-yl)phenoxy]acetyl}glycyl-β-alanine

N-{[4-((2R,3R)-1-(4-chlorophenyl)-3-{[2-(4-chlorophenyl)-2-oxoethyl]thio}-4-oxoazetidin-2-yl)phenoxy]acetyl}glycine (15 mg, 0.026 mmol), tert-butyl b-alaninate hydrochloride 87 mg, 0.038 mmol) and N-methylmorpholine (25 mg, 0.25 mmol) were dissolved in methylene chloride (0.5 ml). TBTU was added and the mixture was stirred at room temperature for 30 min. The solvent was evaporated and the residue was dissolved in formic acid (0.5 ml). The mixture was stirred over night at room temperature and then evaporated under reduced pressure. The residue was dissolved in methanol (0.5 ml). NaBH4 (15 mg, 0.40 mmol) was added. After 15 min stirring a few drops of acetic acid was added. The reaction mixture was evaporated under reduced pressure. The residue was purified by preparative HPLC using acetonitrile/ammonium acetate buffer (40:60) as eluent. Two diastereomers were separated.

Diastereomer 1. ¹H-NMR, 300 MHz, DMSO): 2.33 (t, 2H), 2.89 (d, 2H), 3.69 (d, 2H), 4.31 (d, 1H), 4.51 (s, 2H), 4.74 (t, 1H), 5.04 (d, 1H) 6.97 (d, 2H), 7.20 (d, 2H) 7.30-7.38 (m, 8H), 7.95 (t, 1H), 8.25 (t, 1H).

Diastereomer 2. ¹H-NMR, 300 MHz, DMSO): 2.34 (t, 2H), 2.91 (d, 2H), 3.70 (d, 2H), 4.28 (d, 1H), 4.51 (s, 2H), 4.71 (t, 1H), 5.08 (d, 1H) 6.98 (d, 2H), 7.21 (d, 2H) 7.30-7.38 (m, 8H), 7.94 (t, 1H), 8.24 (t, 1H).

### Example 8

### 1-(N-{[4-((2R,3R)-1-(4-chlorophenyl)-3-{[2-(4-chlorophenyl)-2-hydroxyethyl]thio}-4-oxoazetidin-2-yl)phenoxy]acetyl}glycyl)piperidine-4-carboxylic acid

To a stirred solution of N-{[4-((2R,3R)-1-(4-chlorophenyl)-3-{[2-(4-chlorophenyl)-2-oxoethyl]thio}-4-oxoazetidin-2-yl)phenoxy]acetyl}glycine (10.8 mg, 0.019 mmol) in DMF (1.5 ml) was added N-methylmorpholine (15 μl, 0.14 mmol). After 5 minutes, TBTU (8.4, 0.026 mmol) was added and the reaction mixture was stirred at 30° C. for 1 hour. Piperidine-4-carboxylic acid (3.2 mg, 0.025 mmol) was added and the reaction mixture was stirred at 30° C. for 2.5 hours. The formation of the ketone of the title compound was confirmed, M/z: 682.18 (M−1). Methanol (2 ml) and sodium borohydride (7.1 mg, 0.19 mmol) were added and the resulting mixture was stirred for 15 minutes. 0.1M NH₄OAc buffer (13 ml) was added. The methanol was removed under reduced pressure and the remaining DMF-solution was purified with preparative HPLC on a C8 column. A gradient from 20 to 70% MeCN in 0.1M NH₄OAc buffer was used as eluent. The pure fractions were collected. After lyophilisation, the title compound was obtained. H-NMR (400 MHz, DMSO-d₆): 1.16-1.64 (m, 2H), 1.77 (b, 2H), 2.35-2.43 (m, 1H) 2.66-2.83 (m, 1H) 2.86-2.94 (m, 2H), 2.97-3.11 (m, 1H), 3.64-3.74 (m, 1H), 3.98 (d, 2H), 4.07-4.16 (m, 1H), 4.28 (d, 0.5H), 4.30 (d, 0.5H), 4.53 (s, 2H), 4.67-4.76 (m, 1H), 5.04 (d, 0.5H), 5.07 (d, 0.5M), 6.99 (d, 2H), 7.20 (d, 2H), 7.29-7.40 (m, 8H), 8.05 (t, 1H). M/z: 688.22 (M+1) and 686.21 (M−1).

### Example 9

### 6-({6-[(N-{[4-((2R,3R)-1-(4-fluorophenyl)-3-{[2-(4-fluorophenyl)-2-hydroxyethyl]thio}-4-oxoazetidin-2-yl)phenoxy]acetyl}glycyl)amino]hexanoyl}amino)hexanoic acid

To a stirred solution of N-{[4-((2R,3R)-1-(4-fluorophenyl)-3-{[2-(4-fluorophenyl)-2-oxoethyl]thio}-4-oxoazetidin-2-yl)phenoxy]acetyl}glycine, (13.5 mg, 0.025 mmol) in DMF (2 ml) were added N-methylmorpholine (0.015 μl, 0.14 mmol) and TBTU (10.3 mg, 0.032 mmol). The reaction mixture was stirred at 30° C. for 50 minutes. Additional N-methylmorpholine (15 μl, 0.14 mmol) and TBTU (8.9 mg, 0.028 mmol) were added the mixture was stirred for 25 minutes. 6-aminohexanoic acid (4.4 mg, 0.034 mmol) was added and the reaction mixture was stirred overnight. Analysis with LC-MS showed the presence of the ketone of the title compound; M/z: 767.08 (M+1). Methanol (3 ml) and sodium borohydride (10.7 mg, 0.28 mmol) were added and the reaction mixture was stirred for 20 minutes. 0.1M NH₄OAc buffer (1.5 ml) was added. The methanol was removed under reduced pressure and the remaining DMF-solution was purified with preparative HPLC on a C8 column. A gradient from 20 to 45% MeCN in 0.1M NH₄OAc buffer was used as eluent. The pure fractions were collected. After lyophilisation, the title compound was. H-NMR (400 MHz, DMSO-d₆): 1.12-1.27 (m, 4H), 1.29-1.38 (m, 4H), 1.40-1.50 (m, 4H), 2.00 (t, 2H), 2.09 (t, 2H), 2.86-2.92 (m, 2H), 2.94-3.04 (m, 4H), 3.69 (d, 2H), 4.25 (d, 0.5H), 4.27 (d, 0.5H), 4.52 (s, 2H), 4.67-4.76 (m, 1H), 5.04 (d, 0.5H), 5.06 (d, 0.5H), 6.97 (d, 2H), 7.05-7.18 (m, 4H), 7.20-7.25 (m, 2H), 7.29-7.39 (m, 4H), 7.67-7.76 (m, 1H), 7.94-8.11 (m, 1H), 8.54 (b, 1H). M/z: 769.20 (M+1) and 767.24 (M−1).

### Example 10

### N⁶-(N-{[4-((2R,3R)-1-(4-fluorophenyl)-3-{[2-(4-fluorophenyl)-2-hydroxyethyl]thio}-4-oxoazetidin-2-yl)phenoxy]acetyl}glycyl)-D-lysine

To a 30° C. solution of N-{[4-((2R,3R)-1-(4-fluorophenyl)-3-{[2-(4-fluorophenyl)-2-oxoethyl]thio}-4-oxoazetidin-2-yl)phenoxy]acetyl}glycine (0.030 g, 0.055 mmol) in CH₂Cl_{2 (}5 ml) under an atmosphere of nitrogen was added N-methylmorpholine (0.017 g, 0.166 mmol) followed by the addition of TBTU (0.023 g, 0.072 mmol). After 1.5 h N²-(tert-butoxycarbonyl)-D-lysine (0.027 g, 0.111 mmol) was added. After 30 minutes full conversion to the corresponding amide had been obtained. The mixture was concentrated and the residue was purified through preparative HPLC using an eluent of 0-50% CH₃CN in 0.1M NH₄OAc buffer. Pure fractions were concentrated. To the residue was added trifluoroacetic acid (4 ml) and CH₂Cl₂ (2 ml). After 10 minutes, full deprotection of the Boc protective group had been achieved. The mixture was concentrated and to the residue was added MeOH (3 ml) and NaBH₄ (0.010 g, 0.277 mmol). Full conversion to the desired alcohol had been obtained within 5 minutes. The mixture was purified through preparative HPLC using an eluent of 20-50% CH₃CN in 0.1M NH₄OAc as eluent. Freeze drying of pure fractions afforded the desired compound. ¹H NMR [(CD₃)₂SO), 400 MHz] δ1.26-1.73 (m, 6H), 2.85-3.09 (m, 5H), 3.70 (d, 2H), 4.25-4.28 (m, 1H), 4.53 (s, 2H), 4.68-4.75 (m, 1H), 5.04-5.07 (m, 1H), 6.97-7.38 (m, 12H), 7.87 (t, 1H), 8.30 (t, 1H).

### Example 11

### N-{2-[(N-{[4-((2R,3R)-1-(4-fluorophenyl)-3-{[2-(4-fluorophenyl)-2-hydroxyethyl]thio}-4-oxoazetidin-2-yl)phenoxy]acetyl}glycyl)amino]ethyl}-D-valine

To a 30° C. solution of N-{[4-((2R,3R)-1-(4-fluorophenyl)-3-{[2-(4-fluorophenyl)-2-oxoethyl]thio}-4-oxoazetidin-2-yl)phenoxy]acetyl}glycine (0.025 g, 0.046 mmol) in CH₂Cl₂ (5 ml) was added N-methylmorpholine (0.014 g, 0.139 mmol) followed by the addition of TBTU (0.014 g, 0.139 mmol). After 1 h, DMF (2 ml) and N-(2-aminoethyl)-D-valine bis(trifluoroacetate) (0.036 g, 0.092 mmol) were added. The mixture was allowed to stir for 1 h after which the reaction was quenched by the addition of water (1 ml). MeOH (3 ml) was added followed by the addition of NaBH₄ (0.035 g, 0.925 mmol). Full conversion to the desired alcohol was achieved within 5 minutes. The mixture was purified through preparative HPLC using an eluent of 0-50% CH₃CN in 0.1 M NH₄OAc buffer. Freeze drying of pure fractions afforded the desired compound. ¹H NMR [(CD₃)₂SO), 400 MHz] δ 0.86 (d, 6H), 1.82-1.92 (m, 1H), 2.50-2.59 (m, 1H), 2.64-2.73 (m, 1H), 2.81-2.94 (m, 3H), 3.14-3.23 (m, 2H), 3.71 (d, 2H), 4.25-4.28 (m, 1H), 4.52 (s, 2H), 4.68-4.75 (m, 1H), 5.04-5.07 (m, 1H), 6.97-7.37 (m, 12H), 8.00 (t, 1H), 8.33 (t, 1H).

### Example 12

### 3[(N-{[4-((2R,3R)-1-(4-fluorophenyl)-3-{[2-(4-fluorophenyl)-2-hydroxyethyl]thio}-4-oxoazetidin-2-yl)phenoxy]acetyl}glycyl)amino]-4,4-dimethylpentanoic acid

To a 30° C. solution of N-{[4-((2R,3R)-1-(4-fluorophenyl)-3-{[2-(4-fluorophenyl)-2-oxoethyl]thio}-4-oxoazetidin-2-yl)phenoxy]acetyl}glycine (0.035 g, 0.065 mmol in DMF (2 ml) under an atmosphere of nitrogen was added N-methylmorpholine (0.026 g, 0.26 mmol) followed by the addition of TBTU (0.027 g, 0.084 mmol). After 1 h, 3-amino-4,4-dimethylpentanoic acid (0.014 g, 0.097 mmol) was added and the mixture was allowed to stir for 30 minutes. Water (1 ml) and MeOH (2 ml) were added followed by the addition of NaBH₄ (0.037 g, 0.97 mmol). Full conversion to the corresponding alcohol was obtained within 5 minutes. The mixture was purified through preparative HPLC using an eluent of 0-50% CH₃CN in 0.1M NH₄OAc buffer. Freeze drying of pure fractions afforded the desired compound. ¹H NMR [(CD₃)₂SO), 400 MHz] δ 0.78 (s, 9H), 2.07-2.15 (m, 1H), 2.41-2.47 (m, 1H), 2.84-2.93 (m, 2H), 3.66-3.78 (m, 2H), 3.97-4.03 (m, 1H), 4.25-4.28 (m, 1H), 4.51 (s, 2H), 4.68-4.75 (m, 1H), 5.03-5.06 (m, 1H), 6.96-7.37 (m, 12H), 7.68 (d, 1H), 8.12-8.16 (m, 1H).

### Example 13

### N²-{[4-((2R,3R)-1-(4-fluorophenyl)-3-{[2-(4-fluorophenyl)-2-hydroxyethyl]thio}-4-oxoazetidin-2-yl)phenoxy]acetyl}-N-[(1R)-1-(hydroxymethyl)-2,2-dimethylpropyl]glycinamide

To a solution of N-{[4-((2R,3R)-1-(4-fluorophenyl)-3-{[2-(4-fluorophenyl)-2-oxoethyl]thio}-4-oxoazetidin-2-yl)phenoxy]acetyl}glycine (0.040 g, 0.074 mmol) in dichloromethane (3 ml) under an atmosphere of nitrogen was added N-methylmorpholine (0.022 g, 0.22 mmol) followed by the addition of TBTU (0.031 g, 0.096 mmol) and (2R)-2-amino-3,3-dimethylbutan-1-ol (0.013 g, 0.11 mmol). After 1 h, the mixture was concentrated. MeOH (2 ml) and NaBH₄ (0.028 g, 0.74 mmol) were added. Full conversion to the corresponding alcohol was obtained within 5 minutes. The mixture was purified through preparative HPLC using an eluent of 20-80% CH₃CN in 0.1M NH₄OAc buffer. Freeze drying of pure fractions afforded the desired compound. ¹H NMR [(CD₃)₂SO), 400 MHz] δ 0.80 (s, 9H), 2.84-2.94 (m, 2H), 3.25-3.32 (m, 1H), 3.50-3.62 (m, 2H), 3.75-3.85 (m, 2H), 4.24-4.27 (m, 1H), 4.34-4.37 (t, 1H), 4.52 (s, 2H), 4.68-4.76 (m, 1H), 5.03-5.06 (m, 1H), 5.62-5.65 (m, 1H), 6.97-7.44 (m, 12H), 8.15 (t, 1H).

### Example 14

### N-({4-[(2R,3R)-3-{[2-(4-chlorophenyl)-2-hydroxyethyl]thio}-1-(4-fluorophenyl)-4-oxoazetidin-2-yl]phenoxy}acetyl)glycyl-3-cyclohexyl-D-alanylglycyl-3-cyclohexyl-D-alanine

To a stirred solution of {4-[(2R,3R)-3-{[2-(4-chlorophenyl)-2-oxoethyl]thio}-1-(4-fluorophenyl)-4-oxoazetidin-2-yl]phenoxy}acetic (32.3 mg, 0.065 mmol) in DMF (3 ml) were added N-methylmorpholine (25 μl, 0.02) and TBTU (23.1 0.072 mmol). The reaction mixture was stirred at 35° C. for 30 minutes. Glycyl-3-cyclohexyl-D-alanine (AR-H077847:005) (15.1 mg, 0.066 mmol) was added and the reaction mixture was stirred overnight. Methanol (3 ml) and sodium borohydride (28.4 mg, 0.75 mmol) were added and the resulting reaction mixture was stirred for 30 minutes. Ammonium acetate (80 mg) was added. The methanol was removed under reduced pressure and the remaining DMF-solution was purified by preparative HPLC on a C8 column. A gradient from 20 to 55% MeCN in 0.1M NH₄OAc buffer was used as eluent. The pure fractions were collected. Some of the MeCN was removed under reduced pressure. After lyophilisation, the title compound was obtained. H-NMR (400 MHz, DMSO-d₆): 0.68-0.90 (m, 6H), 0.98-1.31 (m, 10H), 1.35-1.69 (m, 10H), 2.88 (m, 2H), 3.64, (b, 2H), 3.77 (d, 2H), 4.09-4.19 (m, 1H), 4.22-4.31 (m, 2H), 4.50 (s, 2H), 4.66-4.76 (m, 1H), 5.01 (d, 0.5H), 5.04 (d, 0.5H), 6.96 (d, 2H), 7.08-7.16 (m, 2H), 7.17-7.24 (m, 2H), 7.28-7.37 (m, 6H), 7.77 (b, 1H), 8.02-8.29 (m, 3H). M/z: 921.7 (M−1).

### Example 15

### 1-deoxy-1-[(N-{[4-((2R,3R)-1-(4-fluorophenyl)-3-{[(2R or S)-2-(4-fluorophenyl)-2-hydroxyethyl]thio}-4-oxoazetidin-2-yl)phenoxy]acetyl}glycyl-3-cyclohexyl-D-alanyl)amino]-D-glucitol

N-{[4-((2R,3R)-1-(4-fluorophenyl)-3-{[(2R or S)-2-(4-fluorophenyl)-2-hydroxyethyl]thio}-4-oxoazetidin-2-yl)phenoxy]acetyl}glycyl-3-cyclohexyl-D-alanine (30 mg, 0.043 mol), 4-chlorophenol (7 mg, 0.054 mmol), TBTU (14 mg, 0.043 mmol) and N-methylmorpholine (11 μl, 0.098 mmol) were added to DMF (2 ml) and the reaction mixture was stirred for 2 h.at r.t. To the mixture were added D-glucamin (10 mg, 0.055 mmol) and LiCl (30 mg, 0.7 mmol) and the mixture was stirred for 15 h. at r.t. The reaction mixture was purified by preparative HPLC using acetonitril/ammonium acetate buffer (44:55) as eluent. The collected fractions were lyophilized to obtain the title compound.

(¹H-NMR, 400 MHz, DMSO-d6): 0.7-1.7 (m, 11H), 2.9 (d, 2H), 2.95-3.05 (m, 1H), 3.4-3.6 (m, &H), 3.75 d, 2H), 4.2-4.4 (6H), 4.5 (s, 2H), 4.7 (bs, 2H), 5.05 (s, 1H), 5.6 (bs, 1H), 6.9-7.4 (m, 12H), 7.7-7.8 (m, 1H), 7.95-8.0 (m, 1H), 8.15-8.25 (m, 1H)

### Example 16

### N-[2-({[4-((2R,3R)-1-(4-fluorophenyl)-3-{[2-(4-fluorophenyl)-2-hydroxyethyl]thio}-4-oxoazetidin-2-yl)phenoxy]acetyl}amino)ethyl]-D-valine

The titled compound above was prepared using the same procedure as that used for the synthesis of N-{2-[(N-{[4-((2R,3R)-1-(4-fluorophenyl)-3-{[2-(4-fluorophenyl)-2-hydroxyethyl]thio}-4-oxoazetidin-2-yl)phenoxy]acetyl}glycyl)amino]ethyl}-D-valine but using [4-((2R,3R)-1-(4-fluorophenyl)-3-{[2-(4-fluorophenyl)-2-oxoethyl]thio}-4-oxoazetidin-2-yl)phenoxy]acetic acid instead of N-{[4-((2R,3R)-1-(4-fluorophenyl)-3-{[2-(4-fluorophenyl)-2-oxoethyl]thio}-4-oxoazetidin-2-yl)phenoxy]acetyl}glycine. ¹H NMR [(CD₃)₂SO), 400 MHz] δ 0.85 (d, 3H), 0.87 (d, 3H), 1.83-1.93 (m, 1H), 2.55-2.63 (m, 1H), 2.71-2.78 (m, 1H), 2.84-2.94 (m, 3H), 3.19-3.33 (m, 2H), 4.24-4.27 (m, 1H), 4.46 (s, 2H), 4.68-4.75 (m, 1H), 5.03-5.06 (m, 1H), 6.95-7.37 (m, 12H), 8.10 (t, 1H).

### Example 17

### N-{[4-((2R,3R)-1-(4-fluorophenyl)-3-{[(2R or $)-2-(4-fluorophenyl)-2-hydroxyethyl]thio}-4-oxoazetidin-2-yl)phenoxy]acetyl}glycyl-3-cyclohexyl-N-[2-(trimethylammonio)ethyl]-D-alaninamide acetate

To a stirred solution of N-{[4-((2R,3R)-1-(4-fluorophenyl)-3-{[(2R or S)-2-(4-fluorophenyl)-2-hydroxyethyl]thio}-4-oxoazetidin-2-yl)phenoxy]acetyl}glycyl-3-cyclohexyl-D-alanine (40.1 mg, 0.058 mmol) and 2-amino-N,N,N-trimethylethanaminium chloride hydrochloride (12.7 mg, 0.073 mmol) in DMF (2 ml, dry) was added N-methylmorpholine (20 μl, 0.18 mmol). TBTU (26 mg, 0.081 mmol) was added and the resulting mixture was stirred for 2 hours. The DMF-solution was purified with preparative HPLC on a C8 column. A gradient from 20 to 60% MeCN in 0.1M NH₄OAc buffer was used as eluent. The pure fractions were collected and some of the MeCN was removed under reduced pressure. After lyophilisation, the title compound was obtained. The counter ion, the acetate, emerges from the buffer used during the purification step. H-NMR (500 MHz, DMSO-d₆): 0.74-0.92 (m, 2H), 1.04-1.31 (m, 4H), 1.42-1.51 (m, 2H), 1.55-1.67 (m, 8H), 2.90-2.95 (m, 2H), 3.06 (s, 9H), 3.35-3.52 (m, 4H), 3.69-3.76 (m, 1H), 3.81-3.89 (m, 1H), 4.20-4.26 (m, 1H), 4.26-4.28 (m, 1H), 4.55 (s, 2H), 4.72 (t, 1H), 5.08 (d, 1H), 5.72 (b, 1H), 7.00 (d, 2H), 7.08-7.19 (m, 4H), 7.22-7.27 (m, 2H), 7.32-7.40 (m, 4H), 8.64 (bs, 1H), 9.07 (bd, 2H). M/z: 781 (M+1).

### Example 18

### N-{[4-((2R,3R)-1-(4-fluorophenyl)-3-{[(2R or S)-2-(4-fluorophenyl)-2-hydroxyethyl]thio}-4-oxoazetidin-2-yl)phenoxy]acetyl}glycyl-3-cyclohexyl-N-(2-sulfoethyl)-D-alaninamide

N-{[4-((2R,3R)-1-(4-fluorophenyl)-3-{[(2R or S)-2-(4-fluorophenyl)-2-hydroxyethyl]thio}-4-oxoazetidin-2-yl)phenoxy]acetyl}glycyl-3-cyclohexyl-D-alanine (46 mg, 0.066 mmol), N,N,N-tributylbutan-1-aminium 2-aminoethanesulfonate (31.5 mg, 0.086 mmol) and EDC (16.9 mg, 0.088 mmol) were suspended in DCM (3 ml). DMAP (10.2 mg, 0.83 mmol) was added. DMF (1 ml) was added. The reaction mixture was stirred for 3 hours. The solvent was removed under reduced pressure. The residue was purified with preparative HPLC on a C8 column. A gradient from 20 to 65% MeCN in 0.1M NH₄OAc was used as eluent. The pure fractions were collected, the MeCN was removed under reduced pressure and the remaining water solution was diluted with DCM. The water phase was acidified with KH₂SO₄ (2M) to pH ca 2. The phases were separated using a phase separator. The solvent, from the organic phase, was removed under reduced pressure. The residue was dissolved in MeCN and water. After lyophilisation, the title compound was obtained. H-NMR (400 MHz, DMSO-d₆): 072-1.76 (m, 13H), 2.93 (d, 2H), 3.12-3.28 (m, 2H), 3.74-3.87 (m, 2H), 4.15-4.23 (m, 2H), 4.29 (d, 1H), 4.53 (s, 2H), 4.72 (q, 1H), 5.08 (d, 1H), 5.65 (d, 1H), 6.99-706 (m, 2H), 7.08-7.20 (m, 4H), 7.21-7.28 (m, 2H), 7.31-7.42 (m, 4H), 7.96 (t, 1H), 8.12 (d, 1H), 8.23 (t, 1H).

M/z: 801.59 (M−1).

### Example 19

### N-({4-[(2R,3R)-3-{[2-(4-chloro-3-methylphenyl)-2-hydroxyethyl]thio}-1-(4-fluorophenyl)-4-oxoazetidin-2-yl]phenoxy}acetyl)glycyl-3-cyclohexyl-D-alanine

{4-[(2R,3R)-3-{[2-(4-chloro-3-methylphenyl)-2-oxoethyl]thio}-1-(4-fluorophenyl)-4-oxoazetidin-2-yl]phenoxy}acetic acid (23 mg, 0.045 mmol) was dissolved in DMF (3 ml, dry). N-methylmorpholine (20 μl, 0.18 mmol), TBTU (18.5 mg, 0.058) and 4-chlorophenol (7.5 mg, 0.058 mmol) were added. The reaction mixture was stirred for 2 hours. Glycyl-3-cyclohexyl-D-alanine (13.0 mg, 0.057 mmol) and LiCl (42 mg, 0.99 mmol) were added and the reaction mixture was stirred overnight. The formation of the ketone of the title compound was confirmed; M/z: 722 (M−1) and 724 (M+1).

Methanol (5 ml) and sodium borohydride (32.4 mg, 0.86 mmol) were added and the mixture was stirred for 40 minutes. Ammonium acetate (100 mg) was added. The solvent was removed under reduced pressure and the residue was purified with preparative HPLC on a C8 column. A gradient from 20 to 85% MeCN in 50 mM formic acid/50 mM ammonium formiate buffer was used as eluent. The pure fraction were collected and the MeCN was removed under reduced pressure. The residue was lyophilised to give the title. H-NMR (400 MHz, DMSO-d₆): 0.68-1.72 (m, 13H), 2.22 (s, 1.5H), 2.25 (s, 1.5H), 2.83-2.90 (m, 2H), 3.75 (d, 2H), 4.14-4.23 (m, 1H), 4.24-2.28 (m, 1H), 4.49 (s, 2H), 4.62-4.72 (m, 1H), 5.00 (d, 0.5H), 5.02 (0.5H), 5.67 (b, 1H), 6.96 (d, 2H), 7.09-7.16 (m, 3H), 7.17-7.24 (m, 3H), 7.26-7.30 (m, 1H), 7.31-7.37 (m, 2H), 8.05 (d, 1H), 8.20 (t, 1H).

M/z: 724.55 (M−1).

### Example 20

### 6-[2-(4-{(2R,3R)-1-(4-Fluoro-phenyl)-3-[(R or S)-2-(4-fluoro-phenyl)-2-hydroxy-ethylsulfanyl]-4-oxo-azetidin-2-yl}-phenoxy)-acetylamino]-hexanoic acid

To a stirring solution of (4-{(2R,3R)-1-(4-fluoro-phenyl)-3-[(R or S)-2-(4-fluoro-phenyl)-2-hydroxy-ethylsulfanyl]-4-oxo-azetidin-2-yl}-phenoxy)-acetic acid (50 mg, 0.103 mmol) in DMF (1 mL) at RT was added 4-chlorophenol (17 mg, 0.132 mmol), Et3N (30 μL, 0.217 mmol) and TBTU (39 mg, 0.121 mmol). After 2 h, more TBTU (7 mg, 0.022 mmol) and 4-chlorophenol (4 mg, 0.031 mmol) were added. Full conversion to the p-chlorophenylester was obtained after 4 h. 6-Aminohexanoic acid (16 mg, 0.122 mmol) and lithium chloride (65 mg, 1.53 mmol) were added and the reaction mixture was stirred at RT for 4 days. The reaction was quenched by the addition of water (2 mL) and the resulting solution was purified by preparative HPLC using a gradient of 20-70% MeCN in a 0.1M ammonium acetate buffer as eluent. The purest fractions were pooled and concentrated under reduced pressure. The residue was partitioned between EtOAc and diluted HCl (aq) (pH ˜3) and the aqueous layer was extracted with EtOAc. The combined organic layers was dried over MgSO4 and concentrated. The residue was purified further by flash chromatography on silica gel using a mixture of EtOAc, Heptane and AcOH (85:15+0.1%) as eluent. Concentration of the pure fractions gave the desired product.

¹H-NMR (DMSO, 400 MHz): δ 1.16-1.28 (m, 2H), 1.34-1.52 (m, 4H), 2.16 (t, 2H), 2.91 (d, 2H), 3.04-3.12 (m, 2H), 4.26 (d, 1H), 4.44 (s, 2H), 4.68-4.75 (m, 1H), 5.06 (d, 1H), 5.65 (bs, 1H), 6.92-7.00 (m, 2H), 7.05-7.27 (m, 6H), 7.29-7.41 (m, 4H), 8.01-8.08 (m, 1H), 11.96 (bs, 1H).

### Example 21

### (R)-2-{6-[2-(4-{(2R,3R)-1-(4-Fluoro-phenyl)-3-[(R or S)-2-(4-fluoro-phenyl)-2-hydroxy-ethylsulfanyl]-4-oxo-azetidin-2-yl}-phenoxy)-acetylamino]-hexanoylamino}-3-methyl-butyric acid

To a solution of 6-[2-(4-{(2R,3R)-1-(4-fluoro-phenyl)-3-[(R or S)-2-(4-fluoro-phenyl)-2-hydroxy-ethylsulfanyl]-4-oxo-azetidin-2-yl}-phenoxy)-acetylamino]-hexanoic acid (10 mg, 0.017 mmol) in DMF (1.5 mL) at RT was added NMM (6 μL, 0.059 mmol) and TBTU (7 mg, 0.022 mmol). The mixture was stirred for 30 min before D-Valine (3 mg, 0.026 mmol) was added. The reaction mixture was stirred over the weekend before the reaction was quenched by the addition of water (2 mL). The resulting solution was purified by preparative HPLC using a gradient of 20-40% MeCN in a 0.1M ammonium acetate buffer as eluent. Freeze-drying of the pure fractions gave the title.

¹H-NMR (DMSO, 400 MHz): δ 0.83 (d, 6H), 1.13-1.26 (m, 2H), 1.34-1.52 (m, 4H), 1.95-2.20 (m, 3H), 2.87-2.98 (m, 2H), 3.03-3.12 (m, 2H), 3.98-4.07 (m, 1H), 4.26 (d, 1H), 4.45 (s, 2H), 4.68-4.75 (m, 1H), 5.08 (d, 1H), 5.75 (bs, 1H), 6.92-6.99 (m, 2H), 7.06-7.27 (m, 6H), 7.30-7.40 (m, 4H), 7.62-7.72 (m, 1H), 8.02-8.10 (m, 1H).

### Example 22

### (R)-6-tert-Butoxycarbonylamino-2-{6-[2-(4-{(2R,3R)-1-(4-fluoro-phenyl)-3-[(R or S)-2-(4-fluoro-phenyl)-2-hydroxy-ethylsulfanyl]-4-oxo-azetidin-2-yl}-phenoxy)-acetylamino]-hexanoylamino}-hexanoic acid

To a solution of 6-[2-(4-{(2R,3R)-1-(4-fluoro-phenyl)-3-[(R or S)-2-(4-fluoro-phenyl)-2-hydroxy-ethylsulfanyl]-4-oxo-azetidin-2-yl}-phenoxy)-acetylamino]-hexanoic acid (10 mg, 0.017 mmol) in DMF (1 mL) at RT was added NMM (6 μL, 0.059 mmol) and TBTU (10 mg, 0.031 mmol). The mixture was stirred for 30 min before N⁶-(tert-butoxycarbonyl)-D-lysine (5 mg, 0.020 mmol) was added. The reaction mixture was stirred over the weekend and then purified by preparative HPLC using a gradient of 20-40% MeCN in a 0.1M ammonium acetate buffer as eluent. Freeze-drying of the pure fractions gave the title compound.

¹H-NMR (DMSO, 500 MHz): δ 1.12-1.57 (m, 20H), 1.58-1.69 (m, 1H), 2.08 (t, 2H), 2.82-2.94 (m, 4H), 3.04-3.11 (m, 2H), 4.03-4.13 (m, 1H), 4.26 (d, 1H), 4.44 (s, 2H), 4.68-4.74 (m, 1H), 5.05-5.09 (m, 1H), 5.66 (bs, 1H), 6.71-6.77 (m, 1H), 6.93-6.99 (m, 2H), 7.06-7.19 (m, 4H), 7.20-7.27 (m, 2H), 7.31-7.40 (m, 4H), 7.85-8.08 (m, 2H), 12.43 (bs, 1H).

### Example 23

### (R)-6-Amino-2-{6-[2-(4-{(2R,3R)-1-(4-fluoro-phenyl)-3-[2-(4-fluoro-phenyl)-2-hydroxy-ethylsulfanyl]-4-oxo-azetidin-2-yl}-phenoxy)-acetylamino]-hexanoylamino}-hexanoic acid

(R)-6-tert-Butoxycarbonylamino-2-{6-[2-(4-{(2R,3R)-1-(4-fluoro-phenyl)-3-[(R or S)-2-(4-fluoro-phenyl)-2-hydroxy-ethylsulfanyl]-4-oxo-azetidin-2-yl}-phenoxy)-acetylamino]-hexanoylamino}-hexanoic acid (H117503)(3.0 mg, 0.004 mmol) was dissolved in a mixture of acetic acid (0.5 mL) and formic acid (0.5 mL). The solution was stirred at 40° C. for 2 h and then purified by preparative HPLC using a gradient of 20-70% MeCN in a 0.1M ammonium acetate buffer as eluent. Freeze-drying of the pure fractions gave the title compound.

¹H-NMR (DMSO, 500 MHz): δ 1.12-1.66 (m, 12H), 2.06 (t, 2H), 2.64-2.72 (m, 2H), 2.88-3.00 (m, 2H), 3.03-3.12 (m, 2H), 3.79-3.85 (m, 1H), 4.24-4.28 (m, 1H), 4.46 (s, 2H), 4.69-4.74 (m, 1H), 5.06-5.12 (m, 1H), 6.93-6.99 (m, 2H), 7.07-7.19 (m, 4H), 7.21-7.29 (m, 3H), 7.30-7.41 (m, 4H), 8.09-8.15 (m, 1H).

### Example 24

### 3-{2-[2-(4-{(2R,3R)-1-(4-Fluoro-phenyl)-3-[(R or S)-2-(4-fluoro-phenyl)-2-hydroxy-ethylsulfanyl]-4-oxo-azetidin-2-yl}-phenoxy)-acetylamino]-acetylamino}-propionic acid

To a stirring solution of [2-(4-{(2R,3R)-1-(4-fluoro-phenyl)-3-[(R or S)-2-(4-fluoro-phenyl)-2-hydroxy-ethylsulfanyl]-4-oxo-azetidin-2-yl}-phenoxy)-acetylamino]-acetic acid (50 mg, 0.092 mmol) in dry DMF (0.8 mL) at RT under N2 was added 4-chloro-3,5-dimethylphenol (22 mg, 0.140 mmol), Et3N (30 μL, 0.217 mmol) and TBTU (36 mg, 0.112 mmol). Full conversion to the 4-chloro-3,5-dimethylphenylester was obtained after 3 h. 3-Amino propionic acid (10 mg, 0.112 mmol) and LiCl (6 mg, 1.42 mmol) were added and the reaction mixture was stirred at 40° C. for 20 h. N-Methylmorpholine (6 μL, 0.059 mmol) and more 3-amino propionic acid (2 mg, 0.022 mmol) were added and the reaction mixture was stirred at 40° C. for 3 days. The reaction was quenched by the addition of a 0.1M ammonium acetate buffer and the resulting mixture was purified by preparative HPLC (Kromasil 100-10-C8 (21.2×250), ACN/H20/FA (45/55/0.1)). Freeze-drying of the pure fractions gave the title compound.

¹H-NMR (DMSO, 500 MHz): δ 2.36 (t, 2H), 2.88-2.96 (m, 2H), 3.20-3.30 (m, 2H), 3.70 (d, 2H), 4.27 (d, 1H), 4.52 (s, 2H), 4.72 (t, 1H), 5.08 (d, 1H), 5.67 (bs, 1H), 6.96-7.02 (m, 2H), 7.06-7.19 (m, 4H), 7.21-7.27 (m, 2H), 7.31-7.41 (m, 4H), 7.91-7.97 (m, 1H), 8.23-8.28 (m, 1H).

### Example 25

### 4-{2-[2-(4-{(2R,3R)-1-(4-Fluoro-phenyl)-3-[(R or S)-2-(4-fluoro-phenyl)-2-hydroxy-ethylsulfanyl]-4-oxo-azetidin-2-yl}-phenoxy)-acetylamino]-acetylamino}-butyric acid

To a stirring solution of [2-(4-{(2R,3R)-1-(4-fluoro-phenyl)-3-[(R or S)-2-(4-fluoro-phenyl)-2-hydroxy-ethylsulfanyl]-4-oxo-azetidin-2-yl}-phenoxy)-acetylamino]-acetic acid (50 mg, 0.092 mmol) in dry DMF (0.8 mL) at RT under N2 was added 4-chloro-3,5-dimethylphenol (22 mg, 0.140 mmol), Et3N (30 μL, 0.217 mmol) and TBTU (36 mg, 0.112 mmol). Full conversion to the 4-chloro-3,5-dimethylphenylester was obtained after 3 h. 4-Aminobutyric acid (12 mg, 0.116 mmol) and LiCl (6 mg, 1.4 mmol) were added and the reaction mixture was stirred at 40° C. for 20 h. N-Methylmorpholine (6 μL, 0.059 mmol) and more 4-aminobutyric acid (2 mg, 0.019 mmol) were added and the reaction mixture was stirred at 40° C. for 3 days. The reaction was quenched by the addition of a 0.1M ammonium acetate buffer and the resulting mixture was purified by preparative HPLC (Kromasil 100-10:C8 (21.2×250), ACN/H20/FA (45/55/0.1)). Freeze-drying of the pure fractions the title compound.

¹H-NMR (DMSO, 500 MHz): δ 1.55-1.66 (m, 2H), 2.14-2.23 (m, 2H), 2.88-2.96 (m, 2H), 3.02-3.10 (m, 2H), 3.71 (d, 2H), 4.27 (d, 1H), 4.53 (s, 2H), 4.72 (t, 1H), 5.08 (d, 1H), 5.66 (bs, 1H), 6.96-7.02 (m, 2H), 7.07-7.19 (m, 4H), 7.21-7.27 (m, 2H), 7.30-7.41 (m, 4H), 7.88-7.96 (m, 1H), 8.22-8.29 (m, 1H).

### Example 26

### 5-{2-[2-(4-{(2R,3R)-1-(4-Fluoro-phenyl)-3-[(R or S)-2-(4-fluoro-phenyl)-2-hydroxy-ethylsulfanyl]-4-oxo-azetidin-2-yl}-phenoxy)-acetylamino]-acetylamino}-pentanoic acid

To a stirring solution of [2-(4-{(2R,3R)-1-(4-fluoro-phenyl)-3-[(R or S)-2-(4-fluoro-phenyl)-2-hydroxy-ethylsulfanyl]-4-oxo-azetidin-2-yl}-phenoxy)-acetylamino]-acetic acid 50 mg, 0.092 mmol) in dry DMF (0.8 mL) at RT under N2 was added 4-chloro-3,5-dimethylphenol (22 mg, 0.140 mmol), Et3N (30 μL, 0.217 mmol) and TBTU (36 mg, 0.112 mmol). Full conversion to the 4-chloro-3,5-dimethylphenylester was obtained after 3 h. 5-Aminovaleric acid (13 mg, 0.111 mmol) and LiCl (6 mg, 1.42 mmol) were added and the reaction mixture was stirred at 40° C. for 20 h. N-Methylmorpholine (6 μL, 0.059 mmol) and more 5-aminovaleric acid (2 mg, 0.017 mmol) were added and the reaction mixture was stirred at 40° C. for 3 days. The reaction was quenched by the addition of a 0.1M ammonium acetate buffer and the resulting mixture was purified by preparative HPLC using 47% MeCN in a 0.1M ammonium acetate buffer as eluent. Freeze-drying of the pure fractions gave the title compound.

¹H-NMR (DMSO, 400 MHz): δ 1.32-1.52 (m, 4H), 2.18 (t, 2H), 2.87-3.08 (m, 4H), 3.70 (d, 2H), 4.27 (d, 1H), 4.52 (s, 2H), 4.68-4.75 (m, 1H), 5.07 (d, 1H), 5.67 (bs, 1H), 6.95-7.03 (m, 2H), 7.06-7.27 (m, 6H), 7.30-7.41 (m, 4H), 8.02-8.08 (m, 1H), 8.19-8.26 (m, 1H).

### Example 27

### 6-{2-[2-(4-{(2R,3R)-1-(4-Fluoro-phenyl)-3-[(R or S)-2-(4-fluoro-phenyl)-2-hydroxy-ethylsulfanyl]-4-oxo-azetidin-2-yl}-phenoxy)-acetylamino]-acetylamino}-hexanoic acid

To a stirring solution of [2-(4-{(2R,3R)-1-(4-fluoro-phenyl)-3-[(R or S)-2-(4-fluoro-phenyl)-2-hydroxy-ethylsulfanyl]-4-oxo-azetidin-2-yl}-phenoxy)-acetylamino]-acetic acid (50 mg; 0.092 mmol) in dry DMF (0.8 mL) at RT under N2 was added 4-chloro-3,5-dimethylphenol (22 mg, 0.140 mmol), Et3N (30 μL, 0.217 mmol) and TBTU (36 mg, 0.112 mmol). Full conversion to the 4-chloro-3,5-dimethylphenylester was obtained after 3 h. 6-Aminohexanoic acid (13 mg, 0.099 mmol) and LiCl (6 mg, 1.42 mmol) were added and the reaction mixture was stirred at 40° C. for 20 h. N-Methylmorpholine (6 μL, 0.059 mmol) and more 6-aminohexanoic acid (2 mg, 0.015 mmol) were added and the reaction mixture was stirred at 40° C. for 3 days. The reaction was quenched by the addition of a 0.1M ammonium acetate buffer and the resulting mixture was purified by preparative HPLC using 47% MeCN in a 0.1M ammonium acetate buffer as eluent. Freeze-drying of the pure fractions gave the title compound.

¹H-NMR (DMSO, 400 MHz): δ 1.16-1.53 (m, 6H), 2.17 (t, 2H), 2.87-3.07 (m, 4H), 3.70 (d, 2H), 4.27 (d, 1H), 4.52 (s, 2H), 4.68-4.75 (m, 1H), 5.07 (d, 1H), 5.65 (bs, 1H), 6.95-7.03 (m, 2H), 7.05-7.27 (m, 6H), 7.30-7.41 (m, 4H), 7.79-7.86 (m, 1H), 8.19-8.25 (m, 1H).

### Starting Material for the Examples Above

### tert-butyl N-{2-[(tert-butoxycarbonyl)amino]ethyl}-D-valinate

To a solution of tert-butyl D-valinate (0.77 g, 4.44 mmol) in MeOH (20 ml) under an atmosphere of nitrogen was added tert-butyl (2-oxoethyl)carbamate (0.70 g, 4.43 mmol). The mixture was allowed to stir over night. NaBH₄ (0.25 g, 6.67 mmol) was added and the mixture was allowed to stir for 15 minutes. The mixture was then purified through preparative HPLC using an eluent of 20-90% CH₃CN in 0.1M NH₄OAc buffer as eluent. Pure fractions were collected and concentrated to give the desired compound. ¹H NMR [(CDCl₃), 400 MHz] δ 0.90-0.94 (m, 6H), 1.43 (s, 9H), 1.45 (s, 9H), 1.81-1.89 (m, 1H), 2.46-2.53 (m, 1H), 2.73-2.80 (m, 2H), 3.05-3.13 (m, 1H), 3.17-3.24 (m, 1H), 4.98 (s, 1H, br).

### N-(2-aminoethyl)-D-valine bis(trifluoroacetate)

To a solution of tert-butyl N-{2-[(tert-butoxycarbonyl)amino]ethyl}-D-valinate (0.69 g, 2.18 mmol) in CH₂Cl₂ (2 ml) was added trifluoroacetic acid (5 g). After 48 h, the mixture was concentrated to give the desired compound. ¹H NMR [(CD₃)₂SO), 400 MHz] δ 0.94 (d, 3H), 1.04 (d, 3H), 2.17-2.28 (m, 1H), 3.12-3.26 (m, 4H), 3.96 (d, 1H).

### (4S)-3-{[(4-Methoxybenzyl)thio]acetyl}-4-phenyl-1,3-oxazolidin-2-one

[(4-Methoxybenzyl)thio]acetic acid (1.3 g, 6.1 mmol) was dissolved in dry CH₂Cl₂ (40 ml) and given 0° C. N,N′-Dicyclohexylcarbodiimide (DCC, 6.1 g, 6.1 mmol) and 4-(dimethylamino)pyridine (DMAP, 1.6 g, 12.9 mmol) were added and the mixture was stirred for 30 minutes. (S)-(+)-4-Phenyl-2-oxazolidinone (1.0 g, 6.1 mol) was added and the mixture was stirred at room temperature for 24 hours. The mixture was filtrated, concentrated under reduced pressure and purified by flash-chromatography (Hex:EtOAc 8:2 then 1:1). This afforded 1.7 g (77%) of the title compound.

¹H-NMR (CDCl₃, 200 MHz): δ 3.46-3.59 (m, 3H), 3.74-3.76 (m, 4H), 4.23-4.28 (m, 1H), 4.68 (t, J=8.8 Hz, 1H), 5.38-5-42 (m, 1H), 6.78 (d, J=8.6 Hz, 2H), 7.14 (d, J=8.6 Hz, 2H), 7.32-7.40 (m, 5H).

### tert-Butyl (4-{(1R)-1-[(4-fluorophenyl)amino]-2-[(4-methoxybenzyl)thio]-3-oxo-3-[(4S)-2-oxo-4-phenyl-1,3-oxazolidin-3-yl]propyl}phenoxy)acetate

TiCl₄ (1M in CH₂Cl₂, 12.6 mL, 12.6 mmol) was added to a solution of tetraisopropyl orthotitanate (1.24 mL, 4.2 mmol) in CH₂Cl₂ (80 mL) held at 0° C. under inert atmosphere. The mixture was stirred for 15 minutes, then (4S)-3-{[(4-methoxybenzyl)thio]acetyl}-4-phenyl-1,3-oxazolidin-2-one (6.0 g, 16.8 mmol) in dry CH₂Cl₂ (60 mL) was added dropwise over 30 minutes and the mixture was stirred for ten minutes. Then tert-butyl (4-{(E)-[(4-fluorophenyl)imino]methyl}phenoxy)acetate (11.1 g, 33.6 mmol) in dry CH₂Cl₂ (60 mL) was added dropwise over 30 minutes, the mixture was given −40° C. and stirred for 20 minutes. Ethyl diisopropyl amine (5.8 mL, 33.6 mmol) in 20 mL CH₂Cl₂ was added dropwise over 20 minutes and the mixture was stirred at −40° C. for 90 minutes. The mixture was then given −78° C., added isopropanol (50 mL) and slowly given room temperature over two hours. H₂O (100 mL) was added and the mixture was stirred for 20 minutes at room temperature and then extracted twice with diethyl ether. The combined organic layer was washed with water, dried (MgSO₄) and concentrated under reduced pressure. The crude product was dissolved in methanol and a precipitate formed. Filtration and drying afforded the title compound.

¹H-NMR (CDCl₃, 200 MHz): δ 1.5 (s, 9H), 3.65 (s, 1H), 3.8 (s, 3H), 4.1 (m, 1H), 4.4-4.6 (m, 4H), 5.0-5.2 (m, 2H), 5.4 (m, 1H), 6.4-6.6 (m, 2H), 6.7-7-4 (m, 15H).

### tert-Butyl (4-{(2R,3R)-1-(4-fluorophenyl)-3-[(4-methoxybenzyl)thio]-4-oxoazetidin-2-yl}phenoxy)acetate

tert-Butyl (4-{(1R)-1-[(4-fluorophenyl)amino]-2-[(4-methoxybenzyl)thio]-3-oxo-3-[(4S)-2-oxo-4-phenyl-1,3-oxazolidin-3-yl]propyl}phenoxy)acetate (Method 4) (9.3 g, 13.5 mmol) was dissolved in dry toluene (500 mL) and heated to 90° C. under inert atmosphere. N,O-Bis(trimethylsilyl)acetamide (BSA, 9.9 mL, 40.6 mmol) was added and the mixture was stirred at 90° C. for one hour. The mixture was then given 45° C. and tetrabutylammonium fluoride (TBAF, 1 g) was added. The mixture was stirred at 45° C. for 24 hours. After cooling, the mixture was concentrated under reduced pressure and purified by flash-chromatography (Hex:EtOAc 6:1 then 5:1 then 4:1). This afforded 2.45 g (36%) of the title compound as a white solid.

¹H-NMR. (CDCl₃, 200 MHz): δ 1.5 (s, 9H), 3.7 (s, 3H), 3.9 (m, 3H), 4.5 (m, 3H), 6.7 (d, 2H), 6.8-7.0 (m, 4H), 7.0-7.2 (m, 6H).

### Method 6

### tert-Butyl (4-{(2R,3R)-1-(4-fluorophenyl)-3-[(3-nitropyridin-2-yl)dithio]-4-oxoazetidin-2-yl}phenoxy)acetate

tert-Butyl (4-{(2R,3R)-1-(4-fluorophenyl)-3-[(4-methoxybenzyl)thio]-4-oxoazetidin-2-yl}phenoxy)acetate (Method 5) (2.54 g, 4.86 mmol) was dissolved in CH₂Cl₂ (60 mL) and given 0° C. under inert atmosphere. 3-Nitro-2-pyridinesulfenyl chloride (1.11 g, 5.82 mmol) was added and the mixture was stirred for two hours at 0° C., the one hour at room temperature. Concentration under reduced pressure and purification by flash-chromatography (Hex:EtOAc 2:1) afforded 1 the title compound.

¹H-NMR (CDCl₃, 200 MHz): δ 1.6 (s. 9H), 4.3 (d, 1H), 4.5 (s, 2H), 5.2 (d, 1H), 6.8-7.0 (m, 4H), 7.1-7.3 (m, 4H), 7.4 (m, 1H) 8.5 (d, 1H), 8.9 (d, 1H).

### {4-[(2R,3R)-3-{[2-(4-chlorophenyl)-2-oxoethyl]thio}-1-(4-fluorophenyl)-4-oxoazetidin-2-yl]phenoxy}acetic acid

To a stirred solution of tert-butyl (4-{(2R,3R)-1-(4-fluorophenyl)-3-[(3-nitropyridin-2-yl)dithio]-4-oxoazetidin-2-yl}phenoxy)acetate (100.2 mg, 0.18 mmol) in acetone (5 ml) and water (1.2 ml) was added triphenylphosphine (49.4 mg, 0.19 mmol). The reaction mixture was stirred for 15 minutes. The solvent was removed under reduced pressure and the crude thiol was dissolved in DCM (15 ml). 2-Bromo-1-(4-chlorophenyl)ethanone (84 mg, 0.36 mmol) and triethylamine (50 μl, 0.36 mmol) were added and the reaction mixture was stirred overnight. Additional triphenylphosphine (15.4 mg, 0.058 mmol), 2-bromo-1-(4-chlorophenyl)ethanone (12 mg, 0.051 mmol) and triethylamine (10 μl, 0.07 mmol) were added and the reaction mixture was stirred for 2 h. The formation of the tert-butylester of the title compound was confirmed, M/z: 556.25 (M+1) and 554.34 (M−1). The solvent was removed under reduced pressure. The residue was dissolved in formic acid (10 ml) and the reaction mixture was stirred for 3.5 hours. The formic acid was co-evaporated with toluene. The residue was purified with preparative HPLC on a C8 column. A gradient from 20 to 55% MeCN in 0.1M NH₄OAc was used as eluent. The pure fractions were collected and the MeCN was removed under reduced pressure. The remaining water solution was diluted with ethyl acetate and the water phase was acidified with KHSO₄ (2M) to pH 2. The phases were separated and the organic phase was washed with brine and dried over sodium sulfate. The solvent was removed under reduced pressure to give an oil, which later was lyophilised to give the title compound. H-NMR (400 MHz, DMSO-d₆): 4.29 (d, 1H), 4.33 (q, 2H), 4.60 (s, 2H), 5.12 (d, 1H), 6.87 (d, 2H), 7.08-7.23 (m, 4H), 7.31 (d, 2H), 7.55 (d, 2H), 7.91 (d, 2H), 12.94 (bs, 1H). M/z: 500.08 (M+1) and 498.17 (M−1).

### N-{[4-((2R,3R)-1-(4-fluorophenyl)-3-{[2-(4-fluorophenyl)-2-oxoethyl]thio}-1-oxoazetidin-2-yl)phenoxy]acetyl}-D-valine

To a stirred solution of [4-((2R,3R)-1-(4-fluorophenyl)-3-{[2-(4-fluorophenyl)-2-oxoethyl]thio}-4-oxoazetidin-2-yl)phenoxy]acetic acid, (50.0 mg, 0.10 mmol) in DCM (2 ml) were added tert-butyl D-valinate hydrochloride (28.4 mg, 0.14 mmol) and N-methylmorpholine (3.0 μl, 0.31 mmol). After 5 minutes TBTU (43.7 mg, 0.14 mmol) was added and the mixture was stirred at ambient temperature overnight. The intermediate tert-butylester of the title compound was confirmed. M/z: 637.1 (M−H). After the removal of the solvent under reduced pressure, the yellow residue was dissolved in formic acid (1.5 ml) and heated at 50° C. for 5 hours. The solvent was evaporated off and the residue was purified by preparative HPLC on a C8 column. A gradient from 20 to 50% MeCN in 0.1 M ammonium acetate buffer was used as eluent. After lyophilisation, the title compound was obtained. 1H-NMR (400 MHz, DMS-d₆): 0.74 (t, 6H), 1.98-2.07 (m, 1H), 3.84 (brs, 1H), 4.32 (d, 1H), 4.35 (s, 1H), 4.36 (s, 1H), 4.50 (brs, 2H), 5.16 (d, 1H), 6.96 (d, 2H), 7.10-7.17 (m, 2H), 7.19-7.24 (m, 2H), 7.31-7.38 (m, 4H), 7.66 (brs, 1H), 7.99-8.04 (m, 2H). M/z: 583.0 (M+H) and 581.0 (M−H).

### Glycyl-3-cyclohexyl-D-alanine

N-(tert-butoxycarbonyl)glycine (2.0 g, 11.4 mmol) and DIPEA (4.0 g, 31 mmol) were dissolved in methylene chloride (25 ml). TBTU (4.1 g, 12.8 mmol) was added and the mixture was stirred for 15 min at room temperature. 3-cyclohexyl-D-alanine (2.1 g, 12.2 mmol), was added and the reaction mixture was stirred over night g room temperature. The reaction mixture was transferred to a separation funnel and was then extracted with a water/acetic acid solution (100 ml 5% acetic acid). The organic layer was separated and evaporated under reduced pressure. The residue was dissolved in formic acid (20 ml) and the mixture was stirred over night at 40° C. The formic acid was removed under reduced pressure. The residue was washed with water (50 ml) and then stirred in aceton (25 ml) for 1 h at room temperature. The solid material was filtered off and washed with aceton (20 ml). The title compound was obtained.

¹H-NMR, 300 MHz, CD3COOD): 0.8-1.9 (m, 13H), 3.9-4.1° (m, 2H), 4.55-4.65 (m, 1H).

### 3-cyclohexyl-3-[(N-{[4-((2R,3R)-1-(4-fluorophenyl)-3-{[2-(4-methoxyphenyl)-2-oxoethyl]thio}-4-oxoazetidin-2-yl)phenoxy]acetyl}glycyl)amino]propanoic acid

A mixture of N-{[4-((2R,3R)-1-(4-fluorophenyl)-3-{[2-(4-methoxyphenyl)-2-oxoethyl]thio}-4-oxoazetidin-2-yl)phenoxy]acetyl}glycine, 0.016 g, 0.029 mmole), N-methylmorpholin (0.013 mL, 0.116 mmole) and 1-hydroxybenzotriazole (0.0039 g, 0.029 mmole) in DMF (2 mL) was stirred, TBTU (0.011 g, 0.034 mmole) was added. The mixture was stirred at 30° C. for 2 hours and 20 minutes under N₂-atmosphere. 3-amino-3-cyclohexylpropanoic acid (0.0064 g, 0.037 mmole) was added and stirred at 30° C. for 1 hour. TBTU (0.005 g, 0.015 mmole) was added and stirred for 1 hour. The solvent was removed under reduced pressure and the residue was purified by preparative HPLC on a Kromasil C8—column using a gradient of 5-100% MeCN in 0.15% trifluoroacetic acid buffer as eluent. After removing the solvent under reduced pressure, the desired product was obtained.

M/z 706.13

### N-{[4-((2R,3R)-1-(4-Fluorophenyl)-3-{[2-(4-fluorophenyl)-2-oxoethyl]thio}-4-oxoazetidin-2-yl)phenoxy]acetyl}glycine

A mixture of [4-((2R,3R)-1-(4-fluorophenyl)-3-{[2-(4-methoxyphenyl)-2-oxoethyl]thio}-4-oxoazetidin-2-yl)phenoxy]acetic acid (0.0153 g, 0.031 mmol), tert-butyl glycyl-D-valinate hydrochloride (0.0099 g, 0.037 mmol) and N-methylmorpholine (0.010 ml, 0.091 mmol) in DCM (2 ml) was stirred at room temperature. TBTU (0.016 g, 0.050 mmol) was added and the mixture was stirred for 3.5 h. Trifluoroacetic acid (0.5 ml) was added and after 3.5 h the solvent was removed under reduced pressure. The residue was purified by preparative HPLC on a Kromasil C8—column using a gradient of 5-100% MeCN in 0.15% trifluoroacetic acid buffer as eluent. The solvent was removed under reduced pressure and the title product was obtained. M/z 652.20.

### {4-[(2R,3R)-3-{[2-(4-chloro-3-methylphenyl)-2-oxoethyl]thio}-1-(4-fluorophenyl)-4-oxoazetidin-2-yl]phenoxy}acetic acid

Methyl (4-{(2R,3R)-1-(4-fluorophenyl)-3-[(3-nitropyridin-2-yl)dithio]-4-oxoazetidin-2-yl}phenoxy)acetate (113 mg, 0.22 mmol) was suspended in acetone (6 ml). Triphenylphosphine (85.7 mg, 0.33 mmol) was added followed by addition of water (0.6 ml). The reaction mixture was stirred for 15 minutes. The solvent was removed under reduced pressure. The crude thiol was dissolved in DCM (8 ml). 2-bromo-1-(4-chloro-3-methylphenyl)ethanone (128.4 mg, 0.52 mmol) and triethylamine (70 μl, 0.50 mmol) were added and the reaction mixture was stirred for 3 hours. Additional 2-bromo-1-(4-chloro-3-methylphenyl)ethanone (23.8 mg, 0.096 mmol), triphenylphosphine (42.6 mg, 0.16 mmol) and triethylamine (30 μl, 0.22 mmol) were added. The reaction mixture was stirred for 1.5 hours. Analysis with LC-MS showed the presence of the methyl ester of the title compound.

M/z: 527 (M−1).

The solvent was removed under reduced pressure and the residue was suspended in MeCN (5 ml). Triethylamine (305 μl, 2.19 mmol), H₂O (250 μl, 13.9 mmol) and lithium chloride (210.2 mg, 4.96 mmol) were added. The reaction mixture was stirred for 1 hour. Additional triethylamine (200 μl, 1.44 mmol), H₂O (100 μl, 5.55 mmol) and lithium chloride (112 mg, 2.64 mmol) were added and the reaction mixture was stirred overnight. Additional MeCN (2 ml), Et3N (400 μl, 2.87 mmol) H2O (200 μl, 11.10 mmol) and lithium chloride (232 mg, 5.47 mmol) were added and the reaction mixture was stirred for 3 hour. The solvent was removed under reduced pressure and the residue was purified with preparative HPLC on a C8 column.

A gradient from 20 to 65% MeCN in 0.1M NH₄OAc buffer was as eluent. The MeCN was removed under reduced pressure. The remaining water solution was diluted with DCM. The water phase was acidified with KHSO₄ (2M) to pH 3. The phases were separated and the solvent from the organic phase was removed under reduced pressure. The residue was dissolved in MeCN and water. After lyophilisation, the title compound was obtained. H-NMR (400 MHz, DMSO-d₆): 2.37 (s, 3H), 4.32 (d, 1H), 4.34 (s, 2H), 4.65 (d, 2H), 5.15 (d, 1H), 6.91 (d, 2H), 7.11-7.25 (m, 4H), 7.35 (d, 2H), 7.55 (d, 2H), 7.75-7.80 (d, 1H), 7.92 (b, 1H).

M/z: 514.24 (M+1) and 512.34 (M−1).

The preparation of [4-((2R,3R)-1-(4-fluorophenyl)-3-{[2-(4-fluorophenyl)-2-oxoethyl]thio}-4-oxoazetidin-2-yl)phenoxy]acetic acid, is described in PCT/SE2004/001960.

It will be appreciated by those skilled in the art that the examples may be modified within the realms of the invention, why the invention is not limited to particular embodiments.

### Absorption

Absorption of the compounds of formula (I) was tested in a Caco-2 cells model (Gastroenterology 1989, 96, 736):

| | Caco value |
|---|---|
| Compound (I) | (10⁻⁶ cm/sec) |
| N-{[4-((2R,3R)-1-(4-fluorophenyl)-3-{[(2R or S)-2- | 0.61 |
| (4-fluorophenyl)-2-hydroxyethyl]thio}-4-oxoazetidin-2- | |
| yl)phenoxy]acetyl}glycyl-3-cyclohexyl-N-[2- | |
| (trimethylammonio)ethyl]-D-alaninamide acetate | |

1. A compound of formula (I): 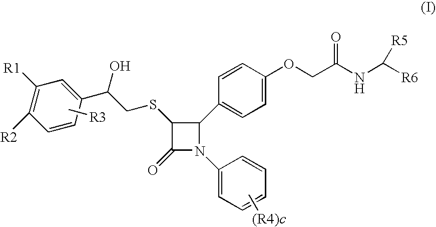
wherein:
R¹ and R² are, independently, hydrogen, C₁₋₆alkyl, C₃₋₆cycloalkyl or aryl; wherein said C₁₋₆alkyl may be optionally substituted by one or more hydroxy, amino, guanidino, carbamoyl, carboxy, C₁₋₆alkoxy, N-(C₁₋₆alkyl)amino, N,N-(C₁₋₆alkyl)₂amino, C₁-C₆alkylcarbonylamino, C₁₋₆alkylS(O)ₐ wherein a is 0-2, C₃₋₆cycloalkyl or aryl; and wherein any aryl group may be optionally substituted by one or two substituents selected from halo, hydroxy, C₁₋₆alkyl of and C₁₋₆alkoxy;
R³ is halo, hydroxy, C₁₋₆alkyl, C₁₋₆alkoxy or C₁₋₆alkylS(O)ₐ wherein a is 0 to 2; wherein R³ is independently optionally substituted on carbon by one or more halo, C₁₋₆alkoxy or hydroxy;
R⁴ is halo, nitro, cyano, hydroxy, amino, carboxy, formyl, carbamoyl, carbamoyloxy, mercapto, sulphamoyl, C₁₋₆alkyl, C₂₋₆alkenyl, C₂₋₆alkenyloxy, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, C₁₋₆alkanoyl-N-(C₁₋₆alkyl)amino, C₁₋₆alkylsulphonylamino, C₁₋₆alkylsulphonyl-N-(C₁₋₆alkyl)amino, N-(C₁₋₆alkyl)carbamoyl, N,N-(C₁₋₆alkyl)₂carbamoyl, N-(C₁₋₆alkyl)carbamoyloxy, N,N-(C₁₋₆alkyl)₂carbamoyloxy, C₁₋₆alkylS(O)ₐ wherein a is 0 to 2, C₁₋₆alkoxycarbonyl, C₁₋₆alkoxycarbonylamino, C₁₋₆alkoxycarbonyl-N-(C₁₋₆alkyl)amino, C₁₋₆alkoxycarbonyloxy, C₁₋₆alkoxycarbonylamino, ureido, N′-(C₁₋₆alkyl)ureido, N-(C₁₋₆alkyl)ureido, N′,N′-(C₁₋₆alkyl)₂ureido, N′-(C₁₋₆alkyl)-N-(C₁₋₆alkyl)ureido, N′,N′-(C₁₋₆alkyl)₂-N-(C₁₋₆alkyl)ureido, N-(C₁₋₆alkyl)sulphamoyl, N,N-(C₁₋₆alkyl)₂sulphamoyl or phenyl; wherein R⁴ is independently optionally substituted on carbon by one or more halo, C₁₋₆alkoxy, hydroxy, amino, carboxy, C₁₋₆alkoxycarbonyl, carbamoyl, N-(C₁₋₆alkyl)carbamoyl, N,N-(C₁₋₆alkyl)₂carbamoyl, C₁₋₆alkanoylamino, C₁₋₆alkanoyl-N-(C₁₋₆alkyl)amino, phenyl, phenoxy, benzoyl, phenylC₁₋₆alkyl or phenylC₁₋₆alkoxy;
R⁵ is hydrogen, halo, nitro, cyano, hydroxy, amino, carbamoyl, mercapto, sulphamoyl, hydroxyaminocarbonyl, C₁₋₁₀alkyl, C₂₋₁₀alkenyl, C₂₋₁₀alkynyl, C₁₋₁₀alkoxy, C₁₋₁₀alkoxycarbonyl, C₁₋₁₀alkanoyl, C₁₋₁₀alkanoyloxy, N-(C₁₋₁₀alkyl)amino, N,N-(C₁₋₁₀alkyl)₂amino, N,N,N-(C₁₋₁₀alkyl)₃ammonio, C₁₋₁₀alkanoylamino, N-(C₁₋₁₀alkyl)carbamoyl, N,N-(C₁₋₁₀alkyl)₂carbamoyl, C₁₋₁₀alkylS(O)ₐ wherein a is 0 to 2, N-(C₁₋₁₀alkyl)sulphamoyl, N,N-(C₁₋₁₀alkyl)₂sulphamoyl, N-(C₁₋₁₀alkyl)sulphamoylamino, N,N-(C₁₋₁₀alkyl)₂sulphamoylamino, C₁₋₁₀alkoxycarbonylamino, carbocyclyl, carbocyclylC₁₋₁₀alkyl, heterocyclyl, heterocyclylC₁₋₁₀alkyl, carbocyclyl-(C₁₋₁₀alkylene)ₑ-R²⁹-(C₁₋₁₀alkylene)_{f}-, heterocyclyl-(C₁₋₁₀alkylene)_{g}-R³⁰-(C₁₋₁₀alkylene)ₕ-, carboxy, sulpho, sulphino, phosphono, —P(O)(OR³¹)(OR³²), —P(O)(OH)(OR³¹), —P(O)(OH)(R³¹) or —P(O)(OR³¹)(R³²) wherein R³¹ and R³² are independently selected from C₁₋₆alkyl; wherein R⁵ may be optionally substituted on carbon by one or more R³³; and wherein if said heterocyclyl contains an —NH— group, that nitrogen may be optionally substituted by R³⁴;
or R⁵ is a group of formula (IA): 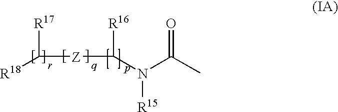
wherein:
Z is —N(R³⁵)—, —N(R³⁵)C(O)—, —O—, or —S(O)ₐ—; wherein a is 0-2 and R³⁵ is hydrogen or C₁₋₄alkyl;
R¹⁵ is hydrogen or C₁₋₄alkyl;
R¹⁶ and R¹⁷ are independently selected from hydrogen, halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, N-(C₁₋₆alkyl)sulphamoyl, N,N-(C₁₋₆alkyl)₂sulphamoyl, carbocyclyl, heterocyclyl, sulpho, sulphino, amidino, phosphono, —P(O)(OR³⁶)(OR³⁷), —P(O)(OH)(OR³⁶), —P(O)(OH)(R³⁶) and —P(O)(OR³⁶)(R³⁷), wherein R³⁶ and R³⁷ are independently selected from C₁₋₆alkyl; wherein R¹⁶ and R¹⁷ may be independently optionally substituted on carbon by one or more R³⁸; and wherein if said heterocyclyl contains an —NH— group, that nitrogen may be optionally substituted by R³⁹;
R¹⁸ is selected from hydrogen, halo, nitro, cyano, hydroxy, amino, carbamoyl, mercapto, sulphamoyl, hydroxyaminocarbonyl, C₁₋₁₀alkyl, C₂₋₁₀alkenyl, C₂₋₁₀alkynyl, C₁₋₁₀alkoxy, C₁₋₁₀alkanoyl, C₁₋₁₀alkanoyloxy, N-(C₁₋₁₀alkyl)amino, N,N-(C₁₋₁₀alkyl)₂amino, C₁₋₁₀alkanoylamino, N-(C₁₋₁₀alkyl)carbamoyl, C₁₋₁₀alkoxycarbonyl, N,N-(C₁₋₁₀alkyl)₂carbamoyl, C₁₋₁₀alkylS(O)ₐ wherein a is 0 to 2, N-(C₁₋₁₀alkyl)sulphamoyl, N,N-(C₁₋₁₀alkyl)₂sulphamoyl, N-(C₁₋₁₀alkyl)sulphamoylamino, N,N-(C₁₋₁₀alkyl)₂sulphamoylamino, carbocyclyl, carbocyclylC₁₋₁₀alkyl, heterocyclyl, heterocyclylC₁₋₁₀alkyl, carbocyclyl-(C₁₋₁₀alkylene)ₑ-R⁴⁰-(C₁₋₁₀alkylene)_{f}- or heterocyclyl-(C₁₋₁₀alkylene)_{g}-R⁴¹-(C₁₋₁₀alkylene)ₕ-, carboxy, sulpho, sulphino, phosphono, —P(O)(OR⁴²)(OR⁴³), —P(O)(OH)(OR⁴²), —P(O)(OH)(R⁴²) and —P(O)(OR⁴²)(R⁴³) wherein R⁴² and R⁴³ are independently selected from C₁₋₆alkyl; wherein R¹⁸ may be optionally substituted on carbon by one or more R⁴⁴; and wherein if said heterocyclyl contains an —NH— group, that nitrogen may be optionally substituted by R⁴⁵;
or R¹⁸ is a group of formula (IB): 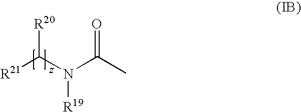
wherein:
R¹⁹ is hydrogen or C₁₋₄alkyl;
R²⁰ is selected from hydrogen, halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, N-(C₁₋₆alkyl)sulphamoyl, N,N-(C₁₋₆alkyl)₂sulphamoyl, carbocyclyl, heterocyclyl, sulpho, sulphino, amidino, phosphono, —P(O)(OR⁴⁶)(OR⁴⁷), —P(O)(OH)(OR⁴⁶), —P(O)(OH)(R⁴⁶) and —P(O)(OR⁴⁶)(R⁴⁷), wherein R⁴⁶ and R⁴⁷ are independently selected from C₁₋₆alkyl; where R²⁰ may be independently optionally substituted on carbon by one or more R⁴⁸; and wherein if said heterocyclyl contains an —NH— group, that nitrogen may be optionally substituted by R⁴⁹;
R²¹ is selected from halo, nitro, cyano, hydroxy, amino, carbamoyl, mercapto, sulphamoyl, hydroxyaminocarbonyl, C₁₋₁₀alkyl, C₂₋₁₀alkenyl, C₂₋₁₀alkynyl, C₁₋₁₀alkoxy, C₁₋₁₀alkoxycarbonyl, C₁₋₁₀alkanoyl, C₁₋₁₀alkanoyloxy, N-(C₁₋₁₀alkyl)amino, N,N-(C₁₋₁₀alkyl)₂amino, N,N,N-(C₁₋₁₀alkyl)₃ammonio, C₁₋₁₀alkanoylamino, N-(C₁₋₁₀alkyl)carbamoyl, N,N-(C₁₋₁₀alkyl)₂carbamoyl, C₁₋₁₀alkylS(O)ₐ wherein a is 0 to 2, N-(C₁₋₁₀alkyl)sulphamoyl, N,N-(C₁₋₁₀alkyl)₂sulphamoyl, N-(C₁₋₁₀alkyl)sulphamoylamino, N,N-(C₁₋₁₀alkyl)₂sulphamoylamino, C₁₋₁₀alkoxycarbonylamino, carbocyclyl, carbocyclylC₁₋₁₀alkyl, heterocyclyl, heterocyclylC₁₋₁₀alkyl, carbocyclyl-(C₁₋₁₀alkylene)ₑ-R⁵⁰-(C₁₋₁₀alkylene)_{f}-, heterocyclyl-(C₁₋₁₀alkylene)_{g}-R⁵¹-(C₁₋₁₀alkylene)ₕ-, carboxy, sulpho, sulphino, phosphono, —P(O)(OR⁵²)(OR⁵³), —P(O)(OH)(OR⁵²), —P(O)(OH)(R⁵²) and —P(O)(OR⁵³)(R⁵³) wherein R⁵² and R⁵³ are independently selected from C₁₋₆alkyl; wherein R²¹ may be independently optionally substituted on carbon by one or more R⁵⁴; and wherein if said heterocyclyl contains an —NH— group, that nitrogen may be optionally substituted by R⁵⁵;
p is 1-3; wherein the values of R¹⁶ may be the same or different;
q is 0-1;
r is 0-3; wherein the values of R¹⁷ may be the same or different;
z is 0-3; wherein the values of R²⁰ may be the same or different;
R³³, R³⁸, R⁴⁴, R⁴⁸ and R⁵⁴ are independently selected from halo, nitro, cyano, hydroxy, amino, carbamoyl, mercapto, sulphamoyl, hydroxyaminocarbonyl, C₁₋₁₀alkyl, C₂₋₁₀alkenyl, C₂₋₁₀alkynyl, C₁₋₁₀alkoxy, C₁₋₁₀alkanoyl, C₁₋₁₀alkanoyloxy, C₁₋₁₀alkoxycarbonyl, N-(C₁₋₁₀alkyl)amino, N,N-(C₁₋₁₀alkyl)₂amino, N,N,N-(C₁₋₁₀alkyl)₃ammonio, C₁₋₁₀alkanoylamino, N-(C₁₋₁₀alkyl)carbamoyl, N,N-(C₁₋₁₀alkyl)₂carbamoyl, C₁₋₁₀alkylS(O)ₐ wherein a is 0 to 2, N-(C₁₋₁₀alkyl)sulphamoyl, N,N-(C₁₋₁₀alkyl)₂sulphamoyl, N-(C₁₋₁₀alkyl)sulphamoylamino, N,N-(C₁₋₁₀alkyl)₂sulphamoylamino, C₁₋₁₀alkoxycarbonylamino, carbocyclyl, carbocyclylC₁₋₁₀alkyl, heterocyclyl, heterocyclylC₁₋₁₀alkyl, carbocyclyl-(C₁₋₁₀alkylene)ₑ-R⁵⁶-(C₁₋₁₀alkylene)_{f}-, heterocyclyl-(C₁₋₁₀alkylene)_{g}-R⁵⁷-(C₁₋₁₀alkylene)ₕ-, carboxy, sulpho, sulphino, amidino, phosphono, —P(O)(OR⁵⁸)(OR⁵⁹), —P(O)(OH)(OR⁵⁸), —P(O)(OH)(R⁵⁸) and —P(O)(OR⁵⁹)(R⁵⁹), wherein R⁵⁸ and R⁵⁹ are independently selected from C₁₋₆alkyl; wherein R³³, R³⁸, R⁴⁴, R⁴⁸ and R⁵⁴ may be independently optionally substituted on carbon by one or more R⁶⁰; and wherein if said heterocyclyl contains an —NH— group, that nitrogen may be optionally substituted by R⁶¹;
R³⁴, R³⁹, R⁴⁵, R⁴⁹, R⁵⁵ and R⁶¹ are independently selected from C₁₋₆alkyl, C₁₋₆alkanoyl, C₁₋₆alkylsulphonyl, sulphamoyl, N-(C₁₋₆alkyl)sulphamoyl, N,N-(C₁₋₆alkyl)₂sulphamoyl, C₁₋₆alkoxycarbonyl, carbamoyl, N-(C₁₋₆alkyl)carbamoyl, N,N-(C₁₋₆alkyl)₂carbamoyl, benzyl, phenethyl, benzoyl, phenylsulphonyl and phenyl;
R²⁹, R³⁰, R⁴⁰, R⁴¹, R⁵⁰, R⁵¹, R⁵⁶ and R⁵⁷ are independently selected from —O—, —NR⁶²—, —S(O)ₓ—, —NR⁶²C(O)NR⁶³—, —NR⁶²C(S)NR⁶³—, —OC(O)N═C—, —NR⁶²C(O)— and —C(O)NR⁶²—; wherein R⁶² and R⁶³ are independently selected from hydrogen and C₁₋₆alkyl, and x is 0-2;
R⁶⁰ is selected from halo, hydroxy, cyano, carbamoyl, ureido, amino, nitro, carboxy, carbamoyl, mercapto, sulphamoyl, trifluoromethyl, trifluoromethoxy, methyl, ethyl, methoxy, ethoxy, vinyl, allyl, ethynyl, methoxycarbonyl, formyl, acetyl, formamido, acetylamino, acetoxy, methylamino, dimethylamino, N-methylcarbamoyl, N,N-dimethylcarbamoyl, methylthio, methylsulphinyl, mesyl, N-methylsulphamoyl and N,N-dimethylsulphamoyl; and
e, f, g and h are independently selected from 0-2;
R⁶ is hydrogen, alkyl, c-alkyl or aryl; and
c is 1, 2, 3, 4 or 5;
or a pharmaceutically acceptable salt, solvate, solvate of such a salt or a prodrug thereof;
provided that compounds of formula (I) are not compounds of formula A 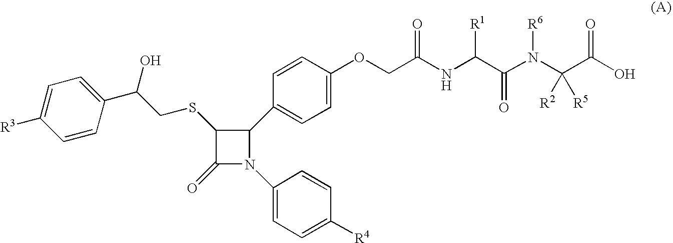
wherein:
R¹ is hydrogen, C₁₋₆alkyl, C₃₋₆cycloalkyl or aryl; wherein said C₁₋₆alkyl may be optionally substituted by one or more hydroxy, amino, guanidino, carbamoyl, carboxy, C₁₋₆alkoxy, N-(C₁₋₆alkyl)amino, N,N-(C₁₋₆alkyl)₂amino, C₁-C₆alkylcarbonylamino, C₁₋₆alkylS(O)ₐ wherein a is 0-2, C₃₋₆cycloalkyl or aryl; and wherein any aryl group may be optionally substituted by one or two substituents selected from halo, hydroxy, C₁₋₆alkyl and C₁₋₆alkoxy;
R² and R⁵ are independently hydrogen, a branched or unbranched C₃₋₆cycloalkyl or aryl; wherein said C₁₋₆alkyl may be optionally substituted by one or more hydroxy, amino, guanidino, cyano, carbamoyl, carboxy, C₁₋₆alkoxy, aryl C₁₋₆alkoxy, (C₁-C₄alkyl)₃Si, N-(C₁₋₆alkyl)amino, N,N-(C₁₋₆alkyl)₂amino, C₁₋₆alkylS(O)ₐ, C₁₋₆alkanoylamino, C₃₋₆cycloalkyl, aryl or aryl C₁₋₆alkylS(O)ₐ, wherein a is 0-2; and wherein any aryl group may be optionally substituted by one or two substituents selected from halo, hydroxy, C₁₋₆alkyl and C₁₋₆alkoxy;
R³ is hydrogen, alkyl, halo, C₁₋₆alkoxy or C₁₋₆alkylS—;
R⁴ is hydrogen, C₁₋₆alkyl, halo or C₁₋₆alkoxy; and
R⁶ is hydrogen, C₁₋₆alkyl, or arylC₁₋₆alkyl;
wherein R⁵ and R² may form a ring with 2-7 carbon atoms and wherein R⁶ and R² may form a ring with 3-6 carbon atoms;
or N-{[4-((2R,3R)-3-{[2-(4-fluorophenyl)-2-hydroxyethyl]thio}-4-oxo-1-phenylazetidin-2-yl)phenoxy]acetyl}glycine.

2. A compound of formula (I2): 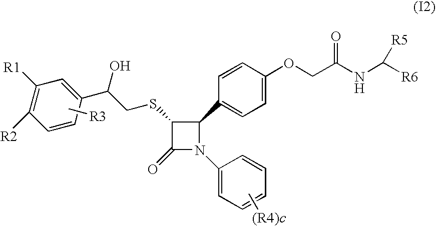
wherein:
R¹ and R² are, independently, hydrogen, C₁₋₆alkyl, C₃₋₆cycloalkyl or aryl; wherein said C₁₋₆alkyl may be optionally substituted by one or more hydroxy, amino, guanidino, carbamoyl, carboxy, C₁₋₆alkoxy, N-(C₁₋₆alkyl)amino, N,N-(C₁₋₆alkyl)₂amino, C₁-C₆alkylcarbonylamino, C₁₋₆alkylS(O)ₐ wherein a is 0-2, C₃₋₆cycloalkyl or aryl; and wherein any aryl group may be optionally substituted by one or two substituents selected from halo, hydroxy, C₁₋₆alkyl and C₁₋₆alkoxy;
R³ is halo, hydroxy, C₁₋₆alkyl, C₁₋₆alkoxy or C₁₋₆alkylS(O)ₐ wherein a is 0 to 2; wherein
R³ is independently optionally substituted on carbon by one or more halo, C₁₋₆alkoxy or hydroxy;
R⁴ is halo, nitro, cyano, hydroxy, amino, carboxy, formyl, carbamoyl, carbamoyloxy, mercapto, sulphamoyl, C₁₋₆alkyl, C₂₋₆alkenyl, C₂₋₆alkenyloxy, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, C₁₋₆alkanoyl-N-(C₁₋₆alkyl)amino, C₁₋₆alkylsulphonylamino, C₁₋₆alkylsulphonyl-N-(C₁₋₆alkyl)amino, N-(C₁₋₆alkyl)carbamoyl, N,N-(C₁₋₆alkyl)₂carbamoyl, N-(C₁₋₆alkyl)carbamoyloxy, N,N-(C₁₋₆alkyl)₂carbamoyloxy, C₁₋₆alkylS(O)ₐ wherein a is 0 to 2, C₁₋₆alkoxycarbonyl, C₁₋₆alkoxycarbonylamino, C₁₋₆alkoxycarbonyl-N-(C₁₋₆alkyl)amino, C₁₋₆alkoxycarbonyloxy, C₁₋₆alkoxycarbonylamino, ureido, N′-(C₁₋₆alkyl)ureido, N-(C₁₋₆alkyl)ureido, N′,N′-(C₁₋₆alkyl)₂ureido, N′-(C₁₋₆alkyl)-N-(C₁₋₆alkyl)ureido, N′,N′-(C₁₋₆alkyl)₂-N-(C₁₋₆alkyl)ureido, N-(C₁₋₆alkyl)sulphamoyl, N,N-(C₁₋₆alkyl)₂sulphamoyl or phenyl; wherein R⁴ is independently optionally substituted on carbon by one or more halo, C₁₋₆alkoxy, hydroxy, amino, carboxy, C₁₋₆alkoxycarbonyl, carbamoyl, N-(C₁₋₆alkyl)carbamoyl, N,N-(C₁₋₆alkyl)₂carbamoyl, C₁₋₆alkanoylamino, C₁₋₆alkanoyl-N-(C₁₋₆alkyl)amino, phenyl, phenoxy, benzoyl, phenylC₁₋₆alkyl or phenylC₁₋₆alkoxy;
R⁵ is hydrogen, halo, nitro, cyano, hydroxy, amino, mercapto, sulphamoyl, hydroxyaminocarbonyl, C₁₋₁₀alkyl, C₂₋₁₀alkenyl, C₂₋₁₀alkynyl, C₁₋₁₀alkoxy, C₁₋₁₀alkoxycarbonyl, C₁₋₁₀alkanoyl, C₁₋₁₀alkanoyloxy, N-(C₁₋₁₀alkyl)amino, N,N-(C₁₋₁₀alkyl)₂amino, N,N,N-(C₁₋₁₀alkyl)₃ammonio, C₁₋₁₀alkanoylamino, C₁₋₁₀alkylS(O)ₐ wherein a is 0 to 2, N-(C₁₋₁₀alkyl)sulphamoyl, N,N-(C₁₋₁₀alkyl)₂sulphamoyl, N-(C₁₋₁₀alkyl)sulphamoylamino, N,N-(C₁₋₁₀alkyl)₂sulphamoylamino, C₁₋₁₀alkoxycarbonylamino, carbocyclyl, carbocyclylC₁₋₁₀alkyl, heterocyclyl, heterocyclylC₁₋₁₀alkyl, carbocyclyl-(C₁₋₁₀alkylene)ₑ-R²⁹-(C₁₋₁₀alkylene)_{f}-, heterocyclyl-(C₁₋₁₀alkylene)_{g}-R³⁰-(C₁₋₁₀alkylene)ₕ-, carboxy, sulpho, sulphino, phosphono, —P(O)(OR³¹)(OR³²), —P(O)(OH)(OR³¹), —P(O)(OH)(R³¹) or —P(O)(OR³¹)(R³²) wherein R³¹ and R³² are independently selected from C₁₋₆alkyl; wherein R⁵ may be optionally substituted on carbon by one or more R³³; and wherein if said heterocyclyl contains an —NH— group, that nitrogen may be optionally substituted by R³⁴;
or R⁵ is a group of formula (IA): 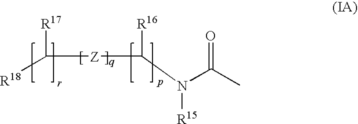
wherein:
Z is —N(R³⁵)—, —N(R³⁵)C(O)—, —O—, or —S(O)ₐ—; wherein a is 0-2 and R³⁵ is hydrogen or C₁₋₄alkyl;
R¹⁵ is hydrogen or C₁₋₄alkyl;
R¹⁶ and R¹⁷ are independently selected from hydrogen, halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, N-(C₁₋₆alkyl)sulphamoyl, N,N-(C₁₋₆alkyl)₂sulphamoyl, carbocyclyl, heterocyclyl, sulpho, sulphino, amidino, phosphono, —P(O)(OR³⁶)(OR³⁷), —P(O)(OH)(OR³⁶), —P(O)(OH)(R³⁶) and —P(O)(OR³⁶)(R³⁷), wherein R³⁶ and R³⁷ are independently selected from C₁₋₆alkyl; wherein R¹⁶ and R¹⁷ may be independently optionally substituted on carbon by one or more R³⁸; and wherein if said heterocyclyl contains an —NH— group, that nitrogen may be optionally substituted by R³⁹;
R¹⁸ is selected from hydrogen, halo, nitro, cyano, hydroxy, amino, carbamoyl, mercapto, sulphamoyl, hydroxyaminocarbonyl, C₁₋₁₀alkyl, C₂₋₁₀alkenyl, C₂₋₁₀alkynyl, C₁₋₁₀alkoxy, C₁₋₁₀alkanoyl, C₁₋₁₀alkanoyloxy, N-(C₁₋₁₀alkyl)amino, N,N-(C₁₋₁₀alkyl)₂amino, C₁₋₁₀alkanoylamino, N-(C₁₋₁₀alkyl)carbamoyl, C₁₋₁₀alkoxycarbonyl, N,N-(C₁₋₁₀alkyl)₂carbamoyl, C₁₋₁₀alkylS(O)ₐ wherein a is 0 to 2, N-(C₁₋₁₀alkyl)sulphamoyl, N,N-(C₁₋₁₀alkyl)₂sulphamoyl, N-(C₁₋₁₀alkyl)sulphamoylamino, N,N-(C₁₋₁₀alkyl)₂sulphamoylamino, carbocyclyl, carbocyclylC₁₋₁₀alkyl, heterocyclyl, heterocyclylC₁₋₁₀alkyl, carbocyclyl-(C₁₋₁₀alkylene)ₑ-R⁴⁰-(C₁₋₁₀alkylene)_{f}-, heterocyclyl-(C₁₋₁₀alkylene)_{g}-R⁴¹-(C₁₋₁₀alkylene)ₕ-, carboxy, sulpho, sulphino, phosphono, —P(O)(OR⁴²)(OR⁴³), —P(O)(OH)(OR⁴²), —P(O)(OH)(R⁴²) and —P(O)(OR⁴²)(R⁴³) wherein R⁴² and R⁴³ are independently selected from C₁₋₆alkyl; wherein R¹⁸ may be optionally substituted on carbon by one or more R⁴⁴; and wherein if said heterocyclyl contains an —NH— group, that nitrogen may be optionally substituted by R⁴⁵;
or R¹⁸ is a group of formula (IB): 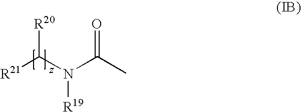
wherein:
R¹⁹ is hydrogen or C₁₋₄alkyl;
R²⁰ is selected from hydrogen, halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, N-(C₁₋₆alkyl)sulphamoyl, N,N-(C₁₋₆alkyl)₂sulphamoyl, carbocyclyl, heterocyclyl, sulpho, sulphino, amidino, phosphono, —P(O)(OR⁴⁶)(OR⁴⁷), —P(O)(OH)(OR⁴⁶), —P(O)(OH)(R⁴⁶) and —P(O)(OR⁴⁶)(R⁴⁷), wherein R⁴⁶ and R⁴⁷ are independently selected from C₁₋₆alkyl; where R²⁰ may be independently optionally substituted on carbon by one or more R⁴⁸; and wherein if said heterocyclyl contains an —NH— group, that nitrogen may be optionally substituted by R⁴⁹;
R²¹ is selected from halo, nitro, cyano, hydroxy, amino, carbamoyl, mercapto, sulphamoyl, hydroxyaminocarbonyl, C₁₋₁₀alkyl, C₂₋₁₀alkenyl, C₂₋₁₀alkynyl, C₁₋₁₀alkoxy, C₁₋₁₀alkoxycarbonyl, C₁₋₁₀alkanoyl, C₁₋₁₀alkanoyloxy, N-(C₁₋₁₀alkyl)amino, N,N-(C₁₋₁₀alkyl)₂amino, N,N,N-(C₁₋₁₀alkyl)₃ammonio, C₁₋₁₀alkanoylamino, N-(C₁₋₁₀alkyl)carbamoyl, N,N-(C₁₋₁₀alkyl)₂carbamoyl, C₁₋₁₀alkylS(O)ₐ wherein a is 0 to 2, N-(C₁₋₁₀alkyl)sulphamoyl, N,N-(C₁₋₁₀alkyl)₂sulphamoyl, N-(C₁₋₁₀alkyl)sulphamoylamino, N,N-(C₁₋₁₀alkyl)₂sulphamoylamino, C₁₋₁₀alkoxycarbonylamino, carbocyclyl, carbocyclylC₁₋₁₀alkyl, heterocyclyl, heterocyclylC₁₋₁₀alkyl, carbocyclyl-(C₁₋₁₀alkylene)ₑ-R⁵⁰-(C₁₋₁₀alkylene)_{f}-, heterocyclyl-(C₁₋₁₀alkylene)_{g}-R⁵¹-(C₁₋₁₀alkylene)ₕ-, carboxy, sulpho, sulphino, phosphono, —P(O)(OR⁵²)(OR⁵³), —P(O)(OH)(OR⁵²), —P(O)(OH)(R⁵²) and —P(O)(OR⁵³)(R⁵³) wherein R⁵² and R⁵³ are independently selected from C₁₋₆alkyl; wherein R²¹ may be independently optionally substituted on carbon by one or more R⁵⁴; and wherein if said heterocyclyl contains an —NH— group, that nitrogen may be optionally substituted by R⁵⁵;
p is 1-3; wherein the values of R¹⁶ may be the same or different;
q is 0-1;
r is 0-3; wherein the values of R¹⁷ may be the same or different;
z is 0-3; wherein the values of R²⁰ may be the same or different;
R³³, R³⁸, R⁴⁴, R⁴⁸ and R⁵⁴ are independently selected from halo, nitro, cyano, hydroxy, amino, carbamoyl, mercapto, sulphamoyl, hydroxyaminocarbonyl, C₁₋₁₀alkyl, C₂₋₁₀alkenyl, C₂₋₁₀alkynyl, C₁₋₁₀alkoxy, C₁₋₁₀alkanoyl, C₁₋₁₀alkanoyloxy, C₁₋₁₀alkoxycarbonyl, N-(C₁₋₁₀alkyl)amino, N,N-(C₁₋₁₀alkyl)₂amino N,N,N-(C₁₋₁₀alkyl)₃ammonio C₁₋₁₀alkanoylamino, N-(C₁₋₁₀alkyl)carbamoyl, N,N-(C₁₋₁₀alkyl)₂carbamoyl, C₁₋₁₀alkylS(O)ₐ wherein a is 0 to 2, N-(C₁₋₁₀alkyl)sulphamoyl, N,N-(C₁₋₁₀alkyl)₂sulphamoyl, N-(C₁₋₁₀alkyl)sulphamoylamino, N,N-(C₁₋₁₀alkyl)₂sulphamoylamino, C₁₋₁₀alkoxycarbonylamino, carbocyclyl, carbocyclylC₁₋₁₀alkyl, heterocyclyl, heterocyclylC₁₋₁₀alkyl, carbocyclyl-(C₁₋₁₀alkylene)ₑ-R⁵⁶-(C₁₋₁₀alkylene)_{f}-, heterocyclyl-(C₁₋₁₀alkylene)_{g}-R⁵⁷-(C₁₋₁₀alkylene)ₕ-, carboxy, sulpho, sulphino, amidino, phosphono, —P(O)(OR⁵⁸)(OR⁵⁹), —P(O)(OH)(OR⁵⁸), —P(O)(OH)(R⁵⁸) and —P(O)(OR⁵⁹)(R⁵⁹), wherein R⁵⁸ and R⁵⁹ are independently selected from C₁₋₆alkyl; wherein R³³, R³⁸, R⁴⁴, R⁴⁸ and R⁵⁴ may be independently optionally substituted on carbon by one or more R⁶⁰; and
wherein if said heterocyclyl contains an —NH— group, that nitrogen may be optionally substituted by R⁶¹;
R³⁴, R³⁹, R⁴⁵, R⁴⁹, R⁵⁵ and R⁶¹ are independently selected from C₁₋₆alkyl, C₁₋₆alkanoyl, C₁₋₆alkylsulphonyl, sulphamoyl, N-(C₁₋₆alkyl)sulphamoyl, N,N-(C₁₋₆alkyl)₂sulphamoyl, C₁₋₆alkoxycarbonyl, carbamoyl, N-(C₁₋₆alkyl)carbamoyl, N,N-(C₁₋₆alkyl)₂carbamoyl, benzyl, phenethyl, benzoyl, phenylsulphonyl and phenyl;
R²⁹, R³⁰, R⁴⁰, R⁴¹, R⁵⁰, R⁵¹, R⁵⁶ and R⁵⁷ are independently selected from —O—, —NR⁶²—, —S(O)ₓ—, —NR⁶²C(O)NR⁶³—, —NR⁶²C(S)NR⁶³—, —OC(O)N═C—, —NR⁶²C(O)— and —C(O)NR⁶²—; wherein R⁶² and R⁶³ are independently selected from hydrogen and C₁₋₆alkyl, and x is 0-2;
R⁶⁰ is selected from halo, hydroxy, cyano, carbamoyl, ureido, amino, nitro, carboxy, carbamoyl, mercapto, sulphamoyl, trifluoromethyl, trifluoromethoxy, methyl, ethyl, methoxy, ethoxy, vinyl, allyl, ethynyl, methoxycarbonyl, formyl, acetyl, formamido, acetylamino, acetoxy, methylamino, dimethylamino, N-methylcarbamoyl, N,N-dimethylcarbamoyl, methylthio, methylsulphinyl, mesyl, N-methylsulphamoyl and N,N-dimethylsulphamoyl; and
e, f, g and h are independently selected from 0-2;
R⁶ is hydrogen, alkyl, c-alkyl or aryl;
c is 1, 2, 3, 4 or 5;
or a pharmaceutically acceptable salt, solvate, solvate of such a salt or a prodrug thereof;
provided that compounds of formula (I2) are not compounds of formula A 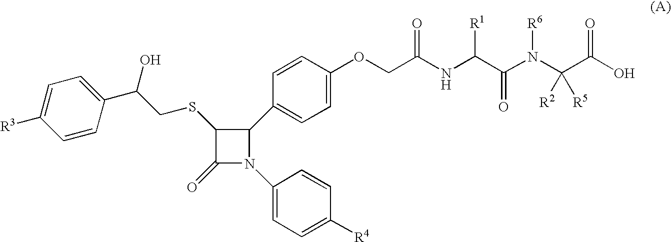
wherein:
R¹ is hydrogen, C₁₋₆alkyl, C₃₋₆cycloalkyl or aryl; wherein said C₁₋₆alkyl may be optionally substituted by one or more hydroxy, amino, guanidino, carbamoyl, carboxy, C₁₋₆alkoxy, N-(C₁₋₆alkyl)amino, N,N-(C₁₋₆alkyl)₂amino, C₁-C₆alkylcarbonylamino, C₁₋₆alkylS(O)ₐ wherein a is 0-2, C₃₋₆cycloalkyl or aryl; and wherein any aryl group may be optionally substituted by one or two substituents selected from halo, hydroxy, C₁₋₆alkyl and C₁₋₆alkoxy;
R² and R⁵ are independently hydrogen, a branched or unbranched C₁₋₆alkyl, C₃₋₆cycloalkyl or aryl; wherein said C₁₋₆alkyl may be optionally substituted by one or more hydroxy, amino, guanidino, cyano, carbamoyl, carboxy, C₁₋₆alkoxy, aryl C₁₋₆alkoxy, (C₁-C₄ alkyl)₃Si, N-(C₁₋₆alkyl)amino, N,N-(C₁₋₆alkyl)₂amino, C₁₋₆alkylS(O)ₐ, C₁₋₆alkanoylamino, C₃₋₆cycloalkyl, aryl or aryl C₁₋₆alkylS(O)ₐ, wherein a is 0-2; and wherein any aryl group may be optionally substituted by one or two substituents selected from halo, hydroxy, C₁₋₆alkyl and C₁₋₆alkoxy;
R³ is hydrogen, alkyl, halo, C₁₋₆alkoxy or C₁₋₆alkylS—;
R⁴ is hydrogen, C₁₋₆alkyl, halo or C₁₋₆alkoxy; and
R⁶ is hydrogen, C₁₋₆alkyl, or arylC₁₋₆alkyl;
wherein R⁵ and R² may form a ring with 2-7 carbon atoms and wherein R⁶ and R² may form a ring with 3-6 carbon atoms;
or N-{[4-((2R,3R)-3-{[2-(4-fluorophenyl)-2-hydroxyethyl]thio}-4-oxo-1-phenylazetidin-2-yl)phenoxy]acetyl}glycine.

3. A compound according to claim 1, wherein: R¹ is hydrogen.

4. A compound according to claim 1, wherein: R² is halo or methoxy.

5. A compound according to claim 1, wherein: R² is chlorine or fluorine.

6. A compound according to claim 1, wherein R¹ and R² form a five-membered ring containing one oxygen.

7. A compound according to claim 1, wherein R¹ and R² form a six-membered ring containing two oxygens.

8. A compound according to claim 1, wherein: R³ is hydrogen.

9. A compound according to claim 1, wherein: R⁴ is halo.

10. A compound according to claim 1, wherein: R⁶ is hydrogen.

11. One or more compounds selected from:
(3R)-3-[(N-{[4-((2R,3R)-1-(4-fluorophenyl)-3-{[2-(4-fluorophenyl)-2-hydroxyethyl]thio}-4-oxoazetidin-2-yl)phenoxy]acetyl}glycyl)amino]-4-phenylbutanoic acid;
3-cyclohexyl-3-[(N-{[4-((2R,3R)-1-(4-fluorophenyl)-3-{[2-hydroxy-2-(4-methoxyphenyl)ethyl]thio}-4-oxoazetidin-2-yl)phenoxy]acetyl}glycyl)amino]propanoic acid;
2-[(N-{[4-((2R,3R)-1-(4-fluorophenyl)-3-{[2-(4-fluorophenyl)-2-hydroxyethyl]thio}-4-oxoazetidin-2-yl)phenoxy]acetyl}glycyl)amino]ethanesulfonic acid;
N⁶-(N-{[4-((2R,3R)-1-(4-fluorophenyl)-3-{[2-(4-fluorophenyl)-2-hydroxyethyl]thio}-4-oxoazetidin-2-yl)phenoxy]acetyl}glycyl)-D-lysine;
N-{2-[(N-{[4-((2R,3R)-1-(4-fluorophenyl)-3-{[2-(4-fluorophenyl)-2-hydroxyethyl]thio}-4-oxoazetidin-2-yl)phenoxy]acetyl}glycyl)amino]ethyl}-D-valine;
3-[(N-{[4-((2R,3R)-1-(4-fluorophenyl)-3-{[2-(4-fluorophenyl)-2-hydroxyethyl]thio}-4-oxoazetidin-2-yl)phenoxy]acetyl}glycyl)amino]-4,4-dimethylpentanoic acid;
N-[2-({[4-((2R,3R)-1-(4-fluorophenyl)-3-{[2-(4-fluorophenyl)-2-hydroxyethyl]thio}-4-oxoazetidin-2-yl)phenoxy]acetyl}amino)ethyl]-D-valine; and
N-{[4-((2R,3R)-1-(4-fluorophenyl)-3-{[(2R or S)-2-(4-fluorophenyl)-2-hydroxyethyl]thio}-4-oxoazetidin-2-yl)phenoxy]acetyl}glycyl-3-cyclohexyl-N-(2-sulfoethyl)-D-alaninamide.

12. A method of treating or preventing hyperlipidemic conditions comprising the administration of an effective amount of a compound according to claim 1 to a mammal in need thereof.

13. A method of treating or preventing atherosclerosis comprising the administration of an effective amount of a compound according to claim 1 to a mammal in need thereof.

14. A method for treating or preventing Alzheimers' disease comprising the administration of an effective amount of a compound according to claim 1 to a mammal in need thereof.

15. A method for treating or preventing cholesterol associated tumors comprising the administration of an effective amount of a compound according to claim 1 to a mammal in need thereof.

16. A pharmaceutical formulation comprising a compound according to claim 1 in admixture with a pharmaceutically acceptable adjuvant, diluent and/or carrier.

17. A combination of a compound according to formula (I) or (I2) 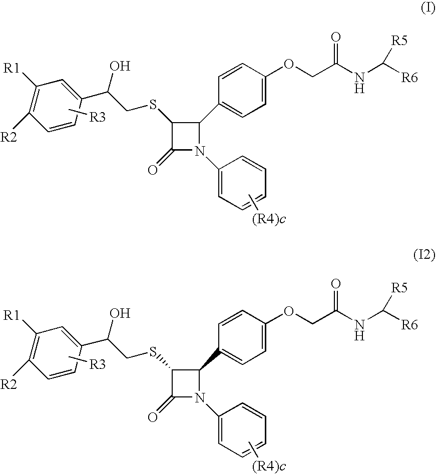
wherein:
R¹ and R² are, independently, hydrogen, C₁₋₆alkyl, C₃₋₆cycloalkyl or aryl; wherein said C₁₋₆alkyl may be optionally substituted by one or more hydroxy, amino, guanidino, carbamoyl, carboxy, C₁₋₆alkoxy, N-(C₁₋₆alkyl)amino, N,N-(C₁₋₆alkyl)₂amino, C₁-C₆alkylcarbonylamino, C₁₋₆alkylS(O)ₐ wherein a is 0-2, C₃₋₆cycloalkyl or aryl; and wherein any aryl group may be optionally substituted by one or two substituents selected from halo, hydroxy, C₁₋₆alkyl and C₁₋₆alkoxy;
R³ is halo, hydroxy, C₁₋₆alkyl, C₁₋₆alkoxy or C₁₋₆alkylS(O)ₐ wherein a is 0 to 2; wherein R³ is independently optionally substituted on carbon by one or more halo, C₁₋₆alkoxy or hydroxy;
R⁴ is halo, nitro, cyano, hydroxy, amino, carboxy, formyl, carbamoyl, carbamoyloxy, mercapto, sulphamoyl, C₁₋₆alkyl, C₂₋₆alkenyl, C₂₋₆alkenyloxy, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, C₁₋₆alkanoyl-N-(C₁₋₆alkyl)amino, C₁₋₆alkylsulphonylamino, C₁₋₆alkylsulphonyl-N-(C₁₋₆alkyl)amino, N-(C₁₋₆alkyl)carbamoyl, N,N-(C₁₋₆alkyl)₂carbamoyl, N-(C₁₋₆alkyl)carbamoyloxy, N,N-(C₁₋₆alkyl)₂carbamoyloxy, C₁₋₆alkylS(O)ₐ wherein a is 0 to 2, C₁₋₆alkoxycarbonyl, C₁₋₆alkoxycarbonylamino, C₁₋₆alkoxycarbonyl-N-(C₁₋₆alkyl)amino, C₁₋₆alkoxycarbonyloxy, C₁₋₆alkoxycarbonylamino, ureido, N′-(C₁₋₆alkyl)ureido, N-(C₁₋₆alkyl)ureido, N′,N′-(C₁₋₆alkyl)₂ureido, N′-(C₁₋₆alkyl)-N-(C₁₋₆alkyl)ureido, N′,N′-(C₁₋₆alkyl)₂-N-(C₁₋₆alkyl)ureido, N-(C₁₋₆alkyl)sulphamoyl, N,N-(C₁₋₆alkyl)₂sulphamoyl or phenyl; wherein R⁴ is independently optionally substituted on carbon by one or more halo, C₁₋₆alkoxy, hydroxy, amino, carboxy, C₁₋₆alkoxycarbonyl, carbamoyl, N-(C₁₋₆alkyl)carbamoyl, N,N-(C₁₋₆alkyl)₂carbamoyl, C₁₋₆alkanoylamino, C₁₋₆alkanoyl-N-(C₁₋₆alkyl)amino, phenyl, phenoxy, benzoyl, phenylC₁₋₆alkyl or phenylC₁₋₆alkoxy;
R⁵ is hydrogen, halo, nitro, cyano, hydroxy, amino, carbamoyl, mercapto, sulphamoyl, hydroxyaminocarbonyl, C₁₋₁₀alkyl, C₂₋₁₀alkenyl, C₂₋₁₀alkynyl, C₁₋₁₀alkoxy, C₁₋₁₀alkoxycarbonyl, C₁₋₁₀alkanoyl, C₁₋₁₀alkanoyloxy, N-(C₁₋₁₀alkyl)amino, N,N-(C₁₋₁₀alkyl)₂amino N,N,N-(C₁₋₁₀alkyl)₃ammonio, C₁₋₁₀alkanoylamino, N-(C₁₋₁₀alkyl)carbamoyl, N,N-(C₁₋₁₀alkyl)₂carbamoyl, C₁₋₁₀alkylS(O)ₐ wherein a is 0 to 2, N-(C₁₋₁₀alkyl)sulphamoyl, N,N-(C₁₋₁₀alkyl)₂sulphamoyl, N-(C₁₋₁₀alkyl)sulphamoylamino, N,N-(C₁₋₁₀alkyl)₂sulphamoylamino, C₁₋₁₀alkoxycarbonylamino, carbocyclyl, carbocyclylC₁₋₁₀alkyl, heterocyclyl, heterocyclylC₁₋₁₀alkyl, carbocyclyl-(C₁₋₁₀alkylene)ₑ-R²⁹-(C₁₋₁₀alkylene)_{f}, heterocyclyl-(C₁₋₁₀alkylene)ₐ-R³⁰-(C₁₋₁₀alkylene)ₕ-, carboxy, sulpho, sulphino, phosphono, —P(O)(OR³¹)(OR³²), —P(O)(OH)(OR³¹), —P(O)(OH)(R³¹) or —P(O)(OR³¹)(R³²) wherein R³¹ and R³² are independently selected from C₁₋₆alkyl; wherein R⁵ may be optionally substituted on carbon by one or more R³³; and wherein if said heterocyclyl contains an —NH— group, that nitrogen may be optionally substituted by R³⁴;
or R⁵ is a group of formula (IA): 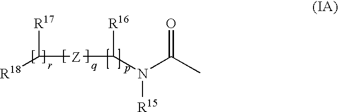
wherein:
Z is —N(R³⁵)—, —N(R³⁵)C(O)—, —O—, or —S(O)ₐ—; wherein a is 0-2 and R³⁵ is hydrogen or C₁₋₄ alkyl;
R¹⁵ is hydrogen or C₁₋₄alkyl;
R¹⁶ and R¹⁷ are independently selected from hydrogen, halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino C₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, N-(C₁₋₆alkyl)sulphamoyl, N,N-(C₁₋₆alkyl)₂sulphamoyl, carbocyclyl, heterocyclyl, sulpho, sulphino, amidino, phosphono, —P(O)(OR³⁶)(OR³⁷), —P(O)(OH)(OR³⁶), —P(O)(OH)(R³⁶) and —P(O)(OR³⁶)(R³⁷), wherein R³⁶ and R³⁷ are independently selected from C₁₋₆alkyl; wherein R¹⁶ and R¹⁷ may be independently optionally substituted on carbon by one or more R³⁸; and wherein if said heterocyclyl contains an —NH— group, that nitrogen may be optionally substituted by R³⁹;
R¹⁸ is selected from hydrogen, halo, nitro, cyano, hydroxy, amino, carbamoyl, mercapto, sulphamoyl, hydroxyaminocarbonyl, C₁₋₁₀alkyl, C₂₋₁₀alkenyl, C₂₋₁₀alkynyl, C₁₋₁₀alkoxy, C₁₋₁₀alkanoyl, C₁₋₁₀alkanoyloxy, N-(C₁₋₁₀alkyl)amino, N,N-(C₁₋₁₀alkyl)₂amino C₁₋₁₀alkanoylamino, N-(C₁₋₁₀alkyl)carbamoyl, C₁₋₁₀alkoxycarbonyl, N,N-(C₁₋₁₀alkyl)₂carbamoyl, C₁₋₁₀alkylS(O)ₐ wherein a is 0 to 2, N-(C₁₋₁₀alkyl)sulphamoyl, N,N-(C₁₋₁₀alkyl)₂sulphamoyl, N-(C₁₋₁₀alkyl)sulphamoylamino, N,N-(C₁₋₁₀alkyl)₂sulphamoylamino, carbocyclyl, carbocyclylC₁₋₁₀alkyl, heterocyclyl, heterocyclylC₁₋₁₀alkyl, carbocyclyl-(C₁₋₁₀alkylene)ₑ-R⁴⁰-(C₁₋₁₀-alkylene)_{f}- or heterocyclyl-(C₁₋₁₀alkylene)ₐ-R⁴¹-(C₁₋₁₀alkylene)ₕ-, carboxy, sulpho, sulphino, phosphono, —P(O)(OR⁴²)(OR⁴³), —P(O)(OH)(OR⁴²), —P(O)(OH)(R⁴²) and —P(O)(OR⁴²)(R⁴³) wherein R⁴² and R⁴³ are independently selected from C₁₋₆alkyl; wherein R¹⁸ may be optionally substituted on carbon by one or more R⁴⁴; and wherein if said heterocyclyl contains an —NH— group, that nitrogen may be optionally substituted by R⁴⁵;
or R¹⁸ is a group of formula (IB): 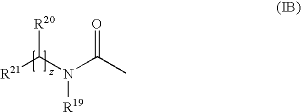
wherein:
R¹⁹ is selected from hydrogen and C₁₋₄alkyl;
R²⁰ is selected from hydrogen, halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino C₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, N-(C₁₋₆alkyl)sulphamoyl, N,N-(C₁₋₆alkyl)₂sulphamoyl, carbocyclyl, heterocyclyl, sulpho, sulphino, amidino, phosphono, —P(O)(OR⁴⁶)(OR⁴⁷), —P(O)(OH)(OR⁴⁶), —P(O)(OH)(R⁴⁶) and —P(O)(OR⁴⁶)(R⁴⁷), wherein R⁴⁶ and R⁴⁷ are independently selected from C₁₋₆alkyl; where R²⁰ may be independently optionally substituted on carbon by one or more R⁴⁸; and wherein if said heterocyclyl contains an —NH— group, that nitrogen may be optionally substituted by R⁴⁹;
R²¹ is selected from halo, nitro, cyano, hydroxy, amino, carbamoyl, mercapto, sulphamoyl, hydroxyaminocarbonyl, C₁₋₁₀alkyl, C₂₋₁₀alkenyl, C₂₋₁₀alkynyl, C₁₋₁₀alkoxy, C₁₋₁₀alkoxycarbonyl, C₁₋₁₀alkanoyl, C₁₋₁₀alkanoyloxy, N-(C₁₋₁₀alkyl)amino, N,N-(C₁₋₁₀alkyl)₂amino, N,N,N-(C₁₋₁₀alkyl)₃ammonio, C₁₋₁₀alkanoylamino, N-(C₁₋₁₀alkyl)carbamoyl, N,N-(C₁₋₁₀alkyl)₂carbamoyl, C₁₋₁₀alkylS(O)ₐ wherein a is 0 to 2, N-(C₁₋₁₀alkyl)sulphamoyl, N,N-(C₁₋₁₀alkyl)₂sulphamoyl, N-(C₁₋₁₀alkyl)sulphamoylamino, N,N-(C₁₋₁₀alkyl)₂sulphamoylamino, C₁₋₁₀alkoxycarbonylamino, carbocyclyl, carbocyclylC₁₋₁₀alkyl, heterocyclyl, heterocyclylC₁₋₁₀alkyl, carbocyclyl-(C₁₋₁₀alkylene)ₑ-R⁵⁹-(C₁₋₁₀alkylene)_{f}-, heterocyclyl-(C₁₋₁₀alkylene)ₐ-R⁵¹-(C₁₋₁₀alkylene)ₕ-, carboxy, sulpho, sulphino, phosphono, —P(O)(OR⁵²)(OR⁵³), —P(O)(OH)(OR⁵²), —P(O)(OH)(R⁵²) and —P(O)(OR⁵³)(R⁵³) wherein R⁵² and R⁵³ are independently selected from C₁₋₆alkyl; wherein R²¹ may be independently optionally substituted on carbon by one or more R⁵⁴; and wherein if said heterocyclyl contains an —NH— group, that nitrogen may be optionally substituted by R⁵⁵;
p is 1-3; wherein the values of R¹⁶ may be the same or different;
q is 0-1;
r is 0-3; wherein the values of R¹⁷ may be the same or different;
z is 0-3; wherein the values of R²⁹ may be the same or different;
R³³, R³⁸, R⁴⁴, R⁴⁸ and R⁵⁴ are independently selected from halo, nitro, cyano, hydroxy, amino, carbamoyl, mercapto, sulphamoyl, hydroxyaminocarbonyl, C₁₋₁₀alkyl, C₂₋₁₀alkenyl, C₂₋₁₀alkynyl, C₁₋₁₀alkoxy, C₁₋₁₀alkanoyl, C₁₋₁₀alkanoyloxy, C₁₋₁₀alkoxycarbonyl, N-(C₁₋₁₀alkyl)amino, N,N-(C₁₋₁₀alkyl)₂amino, N,N,N-(C₁₋₁₀alkyl)₃ammonio, C₁₋₁₀alkanoylamino, N-(C₁₋₁₀alkyl)carbamoyl, N,N-(C₁₋₁₀alkyl)₂carbamoyl, C₁₋₁₀alkylS(O)ₐ wherein a is 0 to 2, N-(C₁₋₁₀alkyl)sulphamoyl, N,N-(C₁₋₁₀alkyl)₂sulphamoyl, N-(C₁₋₁₀alkyl)sulphamoylamino, N,N-(C₁₋₁₀alkyl)₂sulphamoylamino, C₁₋₁₀alkoxycarbonylamino, carbocyclyl, carbocyclylC₁₋₁₀alkyl, heterocyclyl, heterocyclylC₁₋₁₀alkyl, carbocyclyl-(C₁₋₁₀alkylene)ₑ-R⁵⁶-(C₁₋₁₀alkylene)_{f}-, heterocyclyl-(C₁₋₁₀alkylene)_{z}-R⁵⁷-(C₁₋₁₀alkylene)ₕ-, carboxy, sulpho, sulphino, amidino, phosphono, —P(O)(OR⁵⁸)(OR⁵⁹), —P(O)(OH)(OR⁵⁸), —P(O)(OH)(R⁵⁸) and —P(O)(OR⁵⁹)(R⁵⁹), wherein R⁵⁸ and R⁵⁹ are independently selected from C₁₋₆alkyl; wherein R³³, R³⁸, R⁴⁴, R⁴⁸ and R⁵⁴ may be independently optionally substituted on carbon by one or more R⁶⁹; and wherein if said heterocyclyl contains an —NH— group, that nitrogen may be optionally substituted by R⁶¹;
R³⁴, R³⁹, R⁴⁵, R⁴⁹, R⁵⁵ and R⁶¹ are independently selected from C₁₋₆alkyl, C₁₋₆alkanoyl, C₁₋₆alkylsulphonyl, sulphamoyl, N-(C₁₋₆alkyl)sulphamoyl, N-(C₁₋₆alkyl)₂sulphamoyl, C₁₋₆alkoxycarbonyl, carbamoyl, N-(C₁₋₆alkyl)carbamoyl, N,N-(C₁₋₆alkyl)₂carbamoyl, benzyl, phenethyl, benzoyl, phenylsulphonyl and phenyl;
R²⁹, R³⁰, R⁴⁰, R⁴¹, R⁵⁰, R⁵¹, R⁵⁶ and R⁵⁷ are independently selected from —O—, —NR⁶²—, —S(O)ₓ—, —NR⁶²C(O)NR⁶³—, —NR⁶²C(S)NR⁶³—, —OC(O)N═C—, —NR⁶²C(O)— and —C(O)NR⁶²—;
wherein R⁶² and R⁶³ are independently selected from hydrogen and C₁₋₆alkyl, and x is 0-2;
R⁶⁰ is selected from halo, hydroxy, cyano, carbamoyl, ureido, amino, nitro, carboxy, carbamoyl, mercapto, sulphamoyl, trifluoromethyl, trifluoromethoxy, methyl, ethyl, methoxy, ethoxy, vinyl, allyl, ethynyl, methoxycarbonyl, formyl, acetyl, formamido, acetylamino, acetoxy, methylamino, dimethylamino, N-methylcarbamoyl, N,N-dimethylcarbamoyl, methylthio, methylsulphinyl, mesyl, N-methylsulphamoyl and N,N-dimethylsulphamoyl;
e, f, g and h are independently selected from 0-2;
R⁶ is hydrogen, alkyl, c-alkyl or aryl; and
c is 1, 2, 3, 4 or 5;
or a pharmaceutically acceptable salt, solvate, solvate of such a salt or a prodrug thereof;
with a PPAR alpha and/or gamma agonist.

18. A combination of a compound according to formula (I) or (I2) 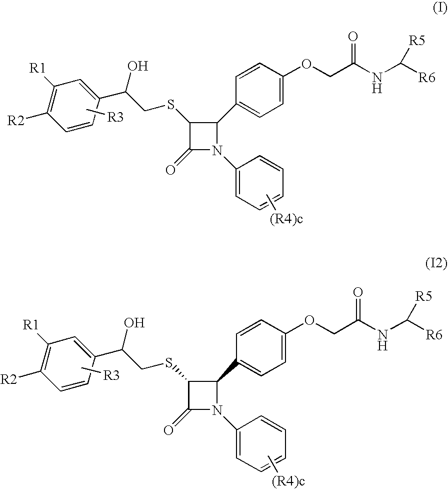
wherein:
R¹ and R² are, independently, hydrogen, C₁₋₆alkyl, C₃₋₆cycloalkyl or aryl; wherein said C₁₋₆alkyl may be optionally substituted by one or more hydroxy, amino, guanidino, carbamoyl, carboxy, C₁₋₆alkoxy, N-(C₁₋₆alkyl)amino, N,N-(C₁₋₆alkyl)₂amino, C₁-C₆alkylcarbonylamino, C₁₋₆alkylS(O)ₐ wherein a is 0-2, C₃₋₆cycloalkyl or aryl; and wherein any aryl group may be optionally substituted by one or two substituents selected from halo, hydroxy, C₁₋₆alkyl and C₁₋₆alkoxy;
R³ is halo, hydroxy, C₁₋₆alkyl, C₁₋₆alkoxy or C₁₋₆alkylS(O)ₐ wherein a is 0 to 2; wherein R³ is independently optionally substituted on carbon by one or more halo, C₁₋₆alkoxy or hydroxy;
R⁴ is halo, nitro, cyano, hydroxy, amino, carboxy, formyl, carbamoyl, carbamoyloxy, mercapto, sulphamoyl, C₁₋₆alkyl, C₂₋₆alkenyl, C₂₋₆alkenyloxy, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, C₁₋₆alkanoyl-N-(C₁₋₆alkyl)amino, C₁₋₆alkylsulphonylamino, C₁₋₆alkylsulphonyl-N-(C₁₋₆alkyl)amino, N-(C₁₋₆alkyl)carbamoyl, N,N-(C₁₋₆alkyl)₂carbamoyl, N-(C₁₋₆alkyl)carbamoyloxy, N,N-(C₁₋₆alkyl)₂carbamoyloxy, C₁₋₆alkylS(O)ₐ wherein a is 0 to 2, C₁₋₆alkoxycarbonyl, C₁₋₆alkoxycarbonylamino, C₁₋₆alkoxycarbonyl-N-(C₁₋₆alkyl)amino, C₁₋₆alkoxycarbonyloxy, C₁₋₆alkoxycarbonylamino, ureido, N′-(C₁₋₆alkyl)ureido, N-(C₁₋₆alkyl)ureido, N′,N′-(C₁₋₆alkyl)₂ureido, N′-(C₁₋₆alkyl)-N-(C₁₋₆alkyl)ureido, N′,N′-(C₁₋₆alkyl)₂-N-(C₁₋₆alkyl)ureido, N-(C₁₋₆alkyl)sulphamoyl, N,N-(C₁₋₆alkyl)₂sulphamoyl or phenyl; wherein R⁴ is independently optionally substituted on carbon by one or more halo, C₁₋₆alkoxy, hydroxy, amino, carboxy, C₁₋₆alkoxycarbonyl, carbamoyl, N-(C₁₋₆alkyl)carbamoyl, N,N-(C₁₋₆alkyl)₂carbamoyl, C₁₋₆alkanoylamino, C₁₋₆alkanoyl-N-(C₁₋₆alkyl)amino, phenyl, phenoxy, benzoyl, phenylC₁₋₆alkyl or phenylC₁₋₆alkoxy;
R⁵ is hydrogen, halo, nitro, cyano, hydroxy, amino, carbamoyl, mercapto, sulphamoyl, hydroxyaminocarbonyl, C₁₋₁₀alkyl, C₂₋₁₀alkenyl, C₂₋₁₀alkynyl, C₁₋₁₀alkoxy, C₁₋₁₀alkoxycarbonyl, C₁₋₁₀alkanoyl, C₁₋₁₀alkanoyloxy, N-(C₁₋₁₀alkyl)amino, N,N-(C₁₋₁₀alkyl)₂amino, N,N,N-(C₁₋₁₀alkyl)₃ammonio, C₁₋₁₀alkanoylamino, N-(C₁₋₁₀alkyl)carbamoyl, N,N-(C₁₋₁₀alkyl)₂carbamoyl, C₁₋₁₀alkylS(O)ₐ wherein a is 0 to 2, N-(C₁₋₁₀alkyl)sulphamoyl, N,N-(C₁₋₁₀alkyl)₂sulphamoyl, N-(C₁₋₁₀alkyl)sulphamoylamino, N,N-(C₁₋₁₀alkyl)₂sulphamoylamino, C₁₋₁₀alkoxycarbonylamino, carbocyclyl, carbocyclylC₁₋₁₀alkyl, heterocyclyl, heterocyclylC₁₋₁₀alkyl, carbocyclyl-(C₁₋₁₀alkylene)ₑ-R²⁹-(C₁₋₁₀alkylene)_{f}-, heterocyclyl-(C₁₋₁₀alkylene)ₐ-R³⁰-(C₁₋₁₀alkylene)ₕ, carboxy, sulpho, sulphino, phosphono, —P(O)(OR³¹)(OR³²), —P(O)(OH)(OR³¹), —P(O)(OH)(R³¹) or —P(O)(OR³¹)(R³²) wherein R³¹ and R³² are independently selected from C₁₋₆alkyl; wherein R⁵ may be optionally substituted on carbon by one or more R³³; and wherein if said heterocyclyl contains an —NH— group, that nitrogen may be optionally substituted by R³⁴;
or R⁵ is a group of formula (IA): 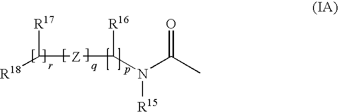
wherein:
Z is —N(R³⁵)—, —N(R³⁵)C(O)—, —O—, or —S(O)ₐ—; wherein a is 0-2 and R³⁵ is hydrogen or C₁₋₄ alkyl;
R¹⁵ is hydrogen or C₁₋₄alkyl;
R¹⁶ and R¹⁷ are independently selected from hydrogen, halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, C₁₋₆alkylS(O), wherein a is 0 to 2, C₁₋₆alkoxycarbonyl, N-(C₁₋₆alkyl)sulphamoyl, N,N-(C₁₋₆alkyl)₂sulphamoyl, carbocyclyl, heterocyclyl, sulpho, sulphino, amidino, phosphono, —P(O)(OR³⁶)(OR³⁷), —P(O)(OH)(OR³⁶), —P(O)(OH)(R³⁶) and —P(O)(OR³⁶)(R³⁷), wherein R³⁶ and R³⁷ are independently selected from C₁₋₆alkyl; wherein R¹⁶ and R¹⁷ may be independently optionally substituted on carbon by one or more R³⁸; and wherein if said heterocyclyl contains an —NH— group, that nitrogen may be optionally substituted by R³⁹;
R¹⁸ is selected from hydrogen, halo, nitro, cyano, hydroxy, amino, carbamoyl, mercapto, sulphamoyl, hydroxyaminocarbonyl, C₁₋₁₀alkyl, C₂₋₁₀alkenyl, C₂₋₁₀alkynyl, C₁₋₁₀alkoxy, C₁₋₁₀alkanoyl, C₁₋₁₀alkanoyloxy, N-(C₁₋₁₀alkyl)amino, N,N-(C₁₋₁₀alkyl)₂amino, C₁₋₁₀alkanoylamino, N-(C₁₋₁₀alkyl)carbamoyl, C₁₋₁₀alkoxycarbonyl, N,N-(C₁₋₁₀alkyl)₂carbamoyl, C₁₋₁₀alkylS(O)ₐ wherein a is 0 to 2, N-(C₁₋₁₀alkyl)sulphamoyl, N,N-(C₁₋₁₀alkyl)₂sulphamoyl, N-(C₁₋₁₀alkyl)sulphamoylamino, N,N-(C₁₋₁₀alkyl)₂sulphamoylamino, carbocyclyl, carbocyclylC₁₋₁₀alkyl, heterocyclyl, heterocyclylC₁₋₁₀alkyl, carbocyclyl-(C₁₋₁₀alkylene)ₑ-R⁴⁰-(C₁₋₁₀-alkylene)_{f}- or heterocyclyl-(C₁₋₁₀alkylene)ₐ-R⁴¹-(C₁₋₁₀alkylene)ₕ-, carboxy, sulpho, sulphino, phosphono, —P(O)(OR⁴²)(OR⁴³), —P(O)(OH)(OR⁴²), —P(O)(OH)(R⁴²) and —P(O)(OR⁴²)(R⁴³) wherein R⁴² and R⁴³ are independently selected from C₁₋₆alkyl; wherein R¹⁸ may be optionally substituted on carbon by one or more R⁴⁴; and wherein if said heterocyclyl contains an —NH— group, that nitrogen may be optionally substituted by R⁴⁵;
or R¹⁸ is a group of formula (IB): 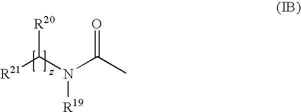
wherein:
R¹⁹ is hydrogen or C₁₋₄alkyl;
R²⁰ is selected from hydrogen, halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, N-(C₁₋₆alkyl)sulphamoyl, N,N-(C₁₋₆alkyl)₂sulphamoyl, carbocyclyl, heterocyclyl, sulpho, sulphino, amidino, phosphono, —P(O)(OR⁴⁶)(OR⁴⁷), —P(O)(OH)(OR⁴⁶), —P(O)(OH)(R⁴⁶) and —P(O)(OR⁴⁶)(R⁴⁷), wherein R⁴⁶ and R⁴⁷ are independently selected from C₁₋₆alkyl; where R²⁰ may be independently optionally substituted on carbon by one or more R⁴⁸;
and wherein if said heterocyclyl contains an —NH— group, that nitrogen may be optionally substituted by R⁴⁹;
R²¹ is selected from halo, nitro, cyano, hydroxy, amino, carbamoyl, mercapto, sulphamoyl, hydroxyaminocarbonyl, C₁₋₁₀alkyl, C₂₋₁₀alkenyl, C₂₋₁₀alkynyl, C₁₋₁₀alkoxy, C₁₋₁₀alkoxycarbonyl, C₁₋₁₀alkanoyl, C₁₋₁₀alkanoyloxy, N-(C₁₋₁₀alkyl)amino, N,N-(C₁₋₁₀alkyl)₂amino, N,N,N-(C₁₋₁₀alkyl)₃ammonio, C₁₋₁₀alkanoylamino, N-(C₁₋₁₀alkyl)carbamoyl, N,N-(C₁₋₁₀alkyl)₂carbamoyl, C₁₋₁₀alkylS(O)ₐ wherein a is 0 to 2, N-(C₁₋₁₀alkyl)sulphamoyl, N,N-(C₁₋₁₀alkyl)₂sulphamoyl, N-(C₁₋₁₀alkyl)sulphamoylamino, N,N-(C₁₋₁₀alkyl)₂sulphamoylamino, C₁₋₁₀alkoxycarbonylamino, carbocyclyl, carbocyclylC₁₋₁₀alkyl, heterocyclyl, heterocyclylC₁₋₁₀alkyl, carbocyclyl-(C₁₋₁₀alkylene)ₑ-R⁵⁰-(C₁₋₁₀alkylene)_{f}-, heterocyclyl-(C₁₋₁₀alkylene)_{z}-R⁵¹-(C₁₋₁₀alkylene)ₕ-, carboxy, sulpho, sulphino, phosphono, —P(O)(OR⁵²)(OR⁵³), —P(O)(OH)(OR⁵²), —P(O)(OH)(R⁵²) and —P(O)(OR⁵³)(R⁵³) wherein R⁵² and R⁵³ are independently selected from C₁₋₆alkyl; wherein R²¹ may be independently optionally substituted on carbon by one or more R⁵⁴; and wherein if said heterocyclyl contains an —NH— group, that nitrogen may be optionally substituted by R⁵⁵;
p is 1-3; wherein the values of R¹⁶ may be the same or different;
q is 0-1;
r is 0-3; wherein the values of R¹⁷ may be the same or different;
z is 0-3; wherein the values of R²⁰ may be the same or different;
R³³, R³⁸, R⁴⁴, R⁴⁸ and R⁵⁴ are independently selected from halo, nitro, cyano, hydroxy, amino, carbamoyl, mercapto, sulphamoyl, hydroxyaminocarbonyl, C₁₋₁₀alkyl, C₂₋₁₀alkenyl, C₂₋₁₀alkynyl, C₁₋₁₀alkoxy, C₁₋₁₀alkanoyl, C₁₋₁₀alkanoyloxy, C₁₋₁₀alkoxycarbonyl, N-(C₁₋₁₀alkyl)amino, N,N-(C₁₋₁₀alkyl)₂amino, N,N,N-(C₁₋₁₀alkyl)₃ammonio, C₁₋₁₀alkanoylamino, N-(C₁₋₁₀alkyl)carbamoyl, N,N-(C₁₋₁₀alkyl)₂carbamoyl, C₁₋₁₀alkylS(O)ₐ wherein a is 0 to 2, N-(C₁₋₁₀alkyl)sulphamoyl, N,N-(C₁₋₁₀alkyl)₂sulphamoyl, N-(C₁₋₁₀alkyl)sulphamoylamino, N,N-(C₁₋₁₀alkyl)₂sulphamoylamino, C₁₋₁₀alkoxycarbonylamino, carbocyclyl, carbocyclylC₁₋₁₀alkyl, heterocyclyl, heterocyclylC₁₋₁₀alkyl, carbocyclyl-(C₁₋₁₀alkylene)ₑ-R⁵⁶-(C₁₋₁₀alkylene) heterocyclyl-(C₁₋₁₀alkylene)_{z}-R⁵⁷-(C₁₋₁₀alkylene)ₕ-, carboxy, sulpho, sulphino, amidino, phosphono, —P(O)(OR⁵⁸)(OR⁵⁹), —P(O)(OH)(OR⁵⁸), —P(O)(OH)(R⁵⁸) and —P(O)(OR⁵⁹)(R⁵⁹), wherein R⁵⁸ and R⁵⁹ are independently selected from C₁₋₆alkyl; wherein R³³, R³⁸, R⁴⁴, R⁴⁸ and R⁵⁴ may be independently optionally substituted on carbon by one or more R⁶⁹; and wherein if said heterocyclyl contains an —NH— group, that nitrogen may be optionally substituted by R⁶¹;
R³⁴, R³⁹, R⁴⁵, R⁴⁹, R⁵⁵ and R⁶¹ are independently selected from C₁₋₆alkyl, C₁₋₆alkanoyl, C₁₋₆alkylsulphonyl, sulphamoyl, N-(C₁₋₆alkyl)sulphamoyl, N,N-(C₁₋₆alkyl)₂sulphamoyl, C₁₋₆alkoxycarbonyl, carbamoyl, N-(C₁₋₆alkyl)carbamoyl, N,N-(C₁₋₆alkyl)₂carbamoyl, benzyl, phenethyl, benzoyl, phenylsulphonyl and phenyl;
R²⁹, R³⁰, R⁴⁰, R⁴¹, R⁵⁰, R⁵¹, R⁵⁶ and R⁵⁷ are independently selected from —O—, —NR⁶²—, —S(O)ₓ—, —NR⁶²C(O)NR⁶³—, —NR⁶²C(S)NR⁶³—, —OC(O)N═C—, —NR⁶²C(O)— and —C(O)NR⁶²—; wherein R⁶² and R⁶³ are independently selected from hydrogen and C₁₋₆alkyl, and x is 0-2;
R⁶⁹ is selected from halo, hydroxy, cyano, carbamoyl, ureido, amino, nitro, carboxy, carbamoyl, mercapto, sulphamoyl, trifluoromethyl, trifluoromethoxy, methyl, ethyl, methoxy, ethoxy, vinyl, allyl, ethynyl, methoxycarbonyl, formyl, acetyl, formamido, acetylamino, acetoxy, methylamino, dimethylamino, N-methylcarbamoyl, N,N-dimethylcarbamoyl, methylthio, methylsulphinyl, mesyl, N-methylsulphamoyl and N,N-dimethylsulphamoyl;
e, f, g and h are independently selected from 0-2;
R⁶ is hydrogen, alkyl, c-alkyl or aryl; and
c is 1, 2, 3, 4 or 5;
or a pharmaceutically acceptable salt, solvate, solvate of such a salt or a prodrug thereof;
with an HMG Co-A reductase inhibitor.

19. A process for preparing a compound of formula (I) 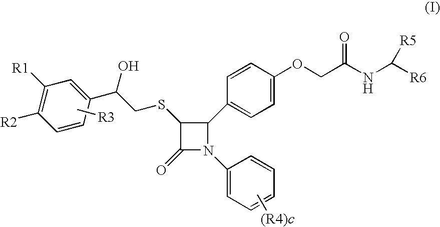
wherein:
R¹ and R² are, independently, hydrogen, C₁₋₆alkyl, C₃₋₆cycloalkyl or aryl; wherein said C₁₋₆alkyl may be optionally substituted by one or more hydroxy, amino, guanidino, carbamoyl, carboxy, C₁₋₆alkoxy, N-(C₁₋₆alkyl)amino, N,N-(C₁₋₆alkyl)₂amino, C₁-C₆alkylcarbonylamino, C₁₋₆alkylS(O)ₐ wherein a is 0-2, C₃₋₆cycloalkyl or aryl; and wherein any aryl group may be optionally substituted by one or two substituents selected from halo, hydroxy, C₁₋₆alkyl and C₁₋₆alkoxy;
R³ is halo, hydroxy, C₁₋₆alkyl, C₁₋₆alkoxy or C₁₋₆alkylS(O)ₐ wherein a is 0 to 2; wherein R³ is independently optionally substituted on carbon by one or more halo, C₁₋₆alkoxy or hydroxy;
R⁴ is halo, nitro, cyano, hydroxy, amino, carboxy, formyl, carbamoyl, carbamoyloxy, mercapto, sulphamoyl, C₁₋₆alkyl, C₂₋₆alkenyl, C₂₋₆alkenyloxy, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, C₁₋₆alkanoyl-N-(C₁₋₆alkyl)amino, C₁₋₆alkylsulphonylamino, C₁₋₆alkylsulphonyl-N-(C₁₋₆alkyl)amino, N-(C₁₋₆alkyl)carbamoyl, N,N-(C₁₋₆alkyl)₂carbamoyl, N-(C₁₋₆alkyl)carbamoyloxy, N,N-(C₁₋₆alkyl)₂carbamoyloxy, C₁₋₆alkylS(O), wherein a is 0 to 2, C₁₋₆alkoxycarbonyl, C₁₋₆alkoxycarbonylamino, C₁₋₆alkoxycarbonyl-N-(C₁₋₆alkyl)amino, C₁₋₆alkoxycarbonyloxy, C₁₋₆alkoxycarbonylamino, ureido, C₁₋₆alkyl)ureido, N-(C₁₋₆alkyl)ureido, N′,N′-(C₁₋₆alkyl)₂ureido, N′-(C₁₋₆alkyl)-N-(C₁₋₆alkyl)ureido, N′,N′-(C₁₋₆alkyl)₂-N-(C₁₋₆alkyl)ureido, N-(C₁₋₆alkyl)sulphamoyl, N,N-(C₁₋₆alkyl)₂sulphamoyl or phenyl; wherein R⁴ is independently optionally substituted on carbon by one or more halo, C₁₋₆alkoxy, hydroxy, amino, carboxy, C₁₋₆alkoxycarbonyl, carbamoyl, N-(C₁₋₆alkyl)carbamoyl, N,N-(C₁₋₆alkyl)₂carbamoyl, C₁₋₆alkanoylamino, C₁₋₆alkanoyl-N-(C₁₋₆alkyl)amino, phenyl, phenoxy, benzoyl, phenylC₁₋₆alkyl or phenylC₁₋₆alkoxy;
R⁵ is hydrogen, halo, nitro, cyano, hydroxy, amino, carbamoyl, mercapto, sulphamoyl, hydroxyaminocarbonyl, C₁₋₁₀alkyl, C₂₋₁₀alkenyl, C₂₋₁₀alkynyl, C₁₋₁₀alkoxy, C₁₋₁₀alkoxycarbonyl, C₁₋₁₀alkanoyl, C₁₋₁₀alkanoyloxy, N-(C₁₋₁₀alkyl)amino, N,N-(C₁₋₁₀alkyl)₂amino, N,N,N-(C₁₋₁₀alkyl)₃ammonio, C₁₋₁₀alkanoylamino, N-(C₁₋₁₀alkyl)carbamoyl, N,N-(C₁₋₁₀alkyl)₂carbamoyl, C₁₋₁₀alkylS(O)ₐ wherein a is 0 to 2, N-(C₁₋₁₀alkyl)sulphamoyl, N,N-(C₁₋₁₀alkyl)₂sulphamoyl, N-(C₁₋₁₀alkyl)sulphamoylamino, N,N-(C₁₋₁₀alkyl)₂sulphamoylamino, C₁₋₁₀alkoxycarbonylamino, carbocyclyl, carbocyclylC₁₋₁₀alkyl, heterocyclyl, heterocyclylC₁₋₁₀alkyl, carbocyclyl-(C₁₋₁₀alkylene)ₑ-R²⁹-(C₁₋₁₀alkylene)_{f}-, heterocyclyl-(C₁₋₁₀alkylene)_{z}-R³⁰-(C₁₋₁₀alkylene)ₕ-, carboxy, sulpho, sulphino, phosphono, —P(O)(OR³¹)(OR³²), —P(O)(OH)(OR³¹), —P(O)(OH)(R³¹) or —P(O)(OR³¹)(R³²) wherein R³¹ and R³² are independently selected from C₁₋₆alkyl; wherein R⁵ may be optionally substituted on carbon by one or more R³³; and wherein if said heterocyclyl contains an —NH— group, that nitrogen may be optionally substituted by R³⁴;
or R⁵ is a group of formula (IA): 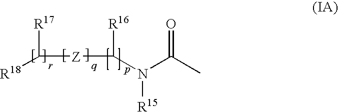
wherein:
Z is —N(R³⁵)—, —N(R³⁵)C(O)—, —O—, or —S(O)ₐ—; wherein a is 0-2 and R³⁵ is hydrogen or C₁₋₄ alkyl;
R¹⁵ is hydrogen or C₁₋₄alkyl;
R¹⁶ and R¹⁷ are independently selected from hydrogen, halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, N-(C₁₋₆alkyl)sulphamoyl, N,N-(C₁₋₆alkyl)₂sulphamoyl, carbocyclyl, heterocyclyl, sulpho, sulphino, amidino, phosphono, —P(O)(OR³⁶)(OR³⁷), —P(O)(OH)(OR³⁶), —P(O)(OH)(R³⁶) and —P(O)(OR³⁶)(R³⁷), wherein R³⁶ and R³⁷ are independently selected from C₁₋₆alkyl; wherein R¹⁶ and R¹⁷ may be independently optionally substituted on carbon by one or more R³⁸; and wherein if said heterocyclyl contains an —NH— group, that nitrogen may be optionally substituted by R³⁹;
R¹⁸ is selected from hydrogen, halo, nitro, cyano, hydroxy, amino, carbamoyl, mercapto, sulphamoyl, hydroxyaminocarbonyl, C₁₋₁₀alkyl, C₂₋₁₀alkenyl, C₂₋₁₀alkynyl, C₁₋₁₀alkoxy, C₁₋₁₀alkanoyl, C₁₋₁₀alkanoyloxy, N-(C₁₋₁₀alkyl)amino, N,N-(C₁₋₁₀alkyl)₂amino C₁₋₁₀alkanoylamino, N-(C₁₋₁₀alkyl)carbamoyl, C₁₋₁₀alkoxycarbonyl, N,N-(C₁₋₁₀alkyl)₂carbamoyl, C₁₋₁₀alkylS(O)ₐ wherein a is 0 to 2, N-(C₁₋₁₀alkyl)sulphamoyl, N,N-(C₁₋₁₀alkyl)₂sulphamoyl, N-(C₁₋₁₀alkyl)sulphamoylamino, N,N-(C₁₋₁₀alkyl)₂sulphamoylamino, carbocyclyl, carbocyclylC₁₋₁₀alkyl, heterocyclyl, heterocyclylC₁₋₁₀alkyl, carbocyclyl-(C₁₋₁₀alkylene)ₑ-R⁴⁰-(C₁₋₁₀-alkylene)_{f}- or heterocyclyl-(C₁₋₁₀alkylene)ₐ-R⁴¹-(C₁₋₁₀alkylene)ₕ-n carboxy, sulpho, sulphino, phosphono, —P(O)(OR⁴²)(OR⁴³), —P(O)(OH)(OR⁴²), —P(O)(OH)(R⁴²) and —P(O)(OR⁴²)(R⁴³) wherein R⁴² and R⁴³ are independently selected from C₁₋₆alkyl; wherein R¹⁸ may be optionally substituted on carbon by one or more R⁴⁴; and wherein if said heterocyclyl contains an —NH— group, that nitrogen may be optionally substituted by R⁴⁵;
or R¹⁸ is a group of formula (IB): 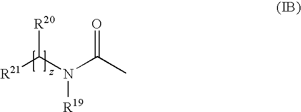
wherein:
R¹⁹ is hydrogen or C₁₋₄alkyl;
R²⁰ is selected from hydrogen, halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, N-(C₁₋₆alkyl)sulphamoyl, N,N-(C₁₋₆alkyl)₂sulphamoyl, carbocyclyl, heterocyclyl, sulpho, sulphino, amidino, phosphono, —P(O)(OR⁴⁶)(OR⁴⁷), —P(O)(OH)(OR⁴⁶), —P(O)(OH)(R⁴⁶) and —P(O)(OR⁴⁶)(R⁴⁷), wherein R⁴⁶ and R⁴⁷ are independently selected from C₁₋₆alkyl; where R²⁰ may be independently optionally substituted on carbon by one or more R⁴⁸;
and wherein if said heterocyclyl contains an —NH— group, that nitrogen may be optionally substituted by R⁴⁹;
R²¹ is selected from halo, nitro, cyano, hydroxy, amino, carbamoyl, mercapto, sulphamoyl, hydroxyaminocarbonyl, C₁₋₁₀alkyl, C₂₋₁₀alkenyl, C₂₋₁₀alkynyl, C₁₋₁₀alkoxy, C₁₋₁₀alkoxycarbonyl, C₁₋₁₀alkanoyl, C₁₋₁₀alkanoyloxy, N-(C₁₋₁₀alkyl)amino, N,N-(C₁₋₁₀alkyl)₂amino, N,N,N-(C₁₋₁₀alkyl)₃ammonio, C₁₋₁₀alkanoylamino, N-(C₁₋₁₀alkyl)carbamoyl, N,N-(C₁₋₁₀alkyl)₂carbamoyl, C₁₋₁₀alkylS(O)ₐ wherein a is 0 to 2, N-(C₁₋₁₀alkyl)sulphamoyl, N,N-(C₁₋₁₀alkyl)₂sulphamoyl, N-(C₁₋₁₀alkyl)sulphamoylamino, N,N-(C₁₋₁₀alkyl)₂sulphamoylamino, C₁₋₁₀alkoxycarbonylamino, carbocyclyl, carbocyclylC₁₋₁₀alkyl, heterocyclyl, heterocyclylC₁₋₁₀alkyl, carbocyclyl-(C₁₋₁₀alkylene)ₑ-R⁵⁰-(C₁₋₁₀alkylene) heterocyclyl-(C₁₋₁₀alkylene)ₐ-R⁵¹-(C₁₋₁₀alkylene)ₕ-, carboxy, sulpho, sulphino, phosphono, —P(O)(OR⁵²)(OR⁵³), —P(O)(OH)(OR⁵²), —P(O)(OH)(R⁵²) and —P(O)(OR⁵³)(R⁵³) wherein R⁵² and R⁵³ are independently selected from C₁₋₆alkyl; wherein R²¹ may be independently optionally substituted on carbon by one or more R⁵⁴; and wherein if said heterocyclyl contains an —NH— group, that nitrogen may be optionally substituted by R⁵⁵;
p is 1-3; wherein the values of R¹⁶ may be the same or different;
q is 0-1;
r is 0-3; wherein the values of R¹⁷ may be the same or different;
z is 0-3; wherein the values of R²⁰ may be the same or different;
R³³, R³⁸, R⁴⁴, R⁴⁸ and R⁵⁴ are independently selected from halo, nitro, cyano, hydroxy, amino, carbamoyl, mercapto, sulphamoyl, hydroxyaminocarbonyl, C₁₋₁₀alkyl, C₂₋₁₀alkenyl, C₂₋₁₀alkynyl, C₁₋₁₀alkoxy, C₁₋₁₀alkanoyl, C₁₋₁₀alkanoyloxy, C₁₋₁₀alkoxycarbonyl, N-(C₁₋₁₀alkyl)amino, N,N-(C₁₋₁₀alkyl)₂amino, N,N,N-(C₁₋₁₀alkyl)₃ammonio, C₁₋₁₀alkanoylamino, N-(C₁₋₁₀alkyl)carbamoyl, N,N-(C₁₋₁₀alkyl)₂carbamoyl, C₁₋₁₀alkylS(O)ₐ wherein a is 0 to 2, N-(C₁₋₁₀alkyl)sulphamoyl, N,N-(C₁₋₁₀alkyl)₂sulphamoyl, N-(C₁₋₁₀alkyl)sulphamoylamino, N,N-(C₁₋₁₀alkyl)₂sulphamoylamino, C₁₋₁₀alkoxycarbonylamino, carbocyclyl, carbocyclylC₁₋₁₀alkyl, heterocyclyl, heterocyclylC₁₋₁₀alkyl, carbocyclyl-(C₁₋₁₀alkylene)ₑ-R⁵⁶-(C₁₋₁₀alkylene)_{f}-, heterocyclyl-(C₁₋₁₀alkylene)ₐ-R⁵⁷-(C₁₋₁₀alkylene)ₕ-, carboxy, sulpho, sulphino, amidino, phosphono, —P(O)(OR⁵⁸)(OR⁵⁹), —P(O)(OH)(OR⁵⁸), —P(O)(OH)(R⁵⁸) and —P(O)(OR⁵⁹)(R⁵⁹), wherein R⁵⁸ and R⁵⁹ are independently selected from C₁₋₆alkyl; wherein R³³, R³⁸, R⁴⁴, R⁴⁸ and R⁵⁴ may be independently optionally substituted on carbon by one or more R⁶⁰; and wherein if said heterocyclyl contains an —NH— group, that nitrogen may be optionally substituted by R⁶¹;
R³⁴, R³⁹, R⁴⁵, R⁴⁹, R⁵⁵ and R⁶¹ are independently selected from C₁₋₆alkyl, C₁₋₆alkanoyl, C₁₋₆alkylsulphonyl, sulphamoyl, N-(C₁₋₆alkyl)sulphamoyl, N,N-(C₁₋₆alkyl)₂sulphamoyl, C₁₋₆alkoxycarbonyl, carbamoyl, N-(C₁₋₆alkyl)carbamoyl, N,N-(C₁₋₆alkyl)₂carbamoyl, benzyl, phenethyl, benzoyl, phenylsulphonyl and phenyl;
R²⁹, R³⁰, R⁴⁰, R⁴¹, R⁵⁰, R⁵¹, R⁵⁶ and R⁵⁷ are independently selected from —O—, —NR⁶²—, —S(O)ₓ—, —NR⁶²C(O)NR⁶³—, —NR⁶²C(S)NR⁶³—, —OC(O)N═C—, —NR⁶²C(O)— and —C(O)NR⁶²—; wherein R⁶² and R⁶³ are independently selected from hydrogen and C₁₋₆alkyl, and x is 0-2;
R⁶⁰ is selected from halo, hydroxy, cyano, carbamoyl, ureido, amino, nitro, carboxy, carbamoyl, mercapto, sulphamoyl, trifluoromethyl, trifluoromethoxy, methyl, ethyl, methoxy, ethoxy, vinyl, allyl, ethynyl, methoxycarbonyl, formyl, acetyl, formamido, acetylamino, acetoxy, methylamino, dimethylamino, N-methylcarbamoyl, N,N-dimethylcarbamoyl, methylthio, methylsulphinyl, mewl, N-methylsulphamoyl and N,N-dimethylsulphamoyl;
e, f, g and h are independently selected from 0-2;
R⁶ is hydrogen, alkyl, c-alkyl or aryl; and
c is 1, 2, 3, 4 or 5;
or a pharmaceutically acceptable salt, solvate, solvate of such a salt or a prodrug thereof which process (wherein variable groups are, unless otherwise specified, as defined in formula (I)) comprising any of the steps of:
Process 1) reacting a compound of formula (II): 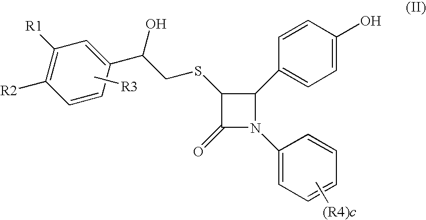
with a compound of formula (III): 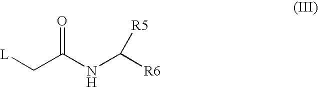
wherein L is a displaceable group; or
Process 2) reacting an acid of formula (IV): 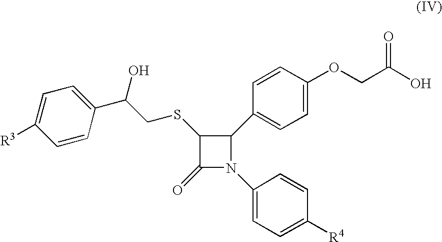
or an activated derivative thereof; with an amine of formula (V): 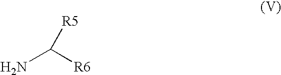
or
Process 3): reducing a compound of formula (VI): 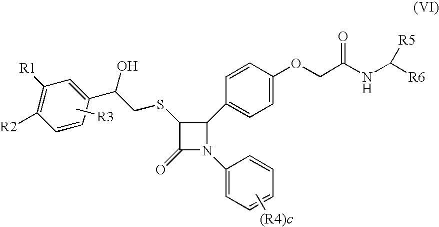
or
Process 4): reacting a compound of formula (VII): 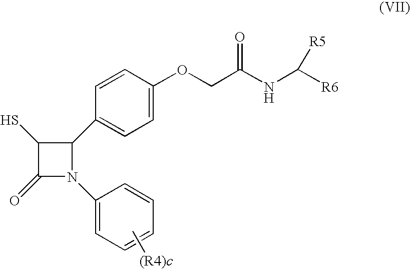
with a compound of formula (VIII): 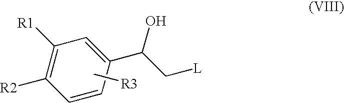
wherein L is a displaceable group; or
Process 5): reacting a compound of formula (IX): 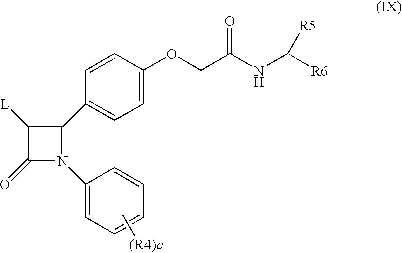
wherein L is a displaceable group; with a compound of formula (X):

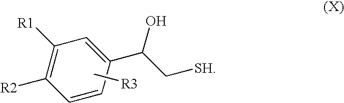

20. A compound according to claim 2, wherein: R¹ is hydrogen.

21. A compound according to claim 2, wherein: R² is halo or methoxy.

22. A compound according to claim 1, wherein: R² is chlorine or fluorine.

23. A compound according to claim 2, wherein R¹ and R² form a five-membered ring containing one oxygen.

24. A compound according to claim 2, wherein R¹ and R² form a six-membered ring containing two oxygens.

25. A compound according to claim 2, wherein: R³ is hydrogen.

26. A compound according to claim 2, wherein: R⁴ is halo.

27. A compound according to claim 2, wherein: R⁶ is hydrogen.

28. A method of treating or preventing hyperlipidemic conditions comprising the administration of an effective amount of a compound according to claim 2 to a mammal in need thereof.

29. A method of treating or preventing atherosclerosis comprising the administration of an effective amount of a compound according to claim 2 to a mammal in need thereof.

30. A method for treating or preventing Alzheimers' disease comprising the administration of an effective amount of a compound according to claim 2 to a mammal in need thereof.

31. A method for treating or preventing cholesterol associated tumors comprising the administration of an effective amount of a compound according to claim 2 to a mammal in need thereof.

32. A pharmaceutical formulation comprising a compound according to claim 2 in admixture with a pharmaceutically acceptable adjuvant, diluent and/or carrier.
